(19) United States
(12) Patent Application Publication
(10) Pub. No.: US 2026/0171190 A1
(43) Pub. Date: Jun. 18, 2026

(54) ASSESSMENT OF GR CELLULAR SIGNALING PATHWAY ACTIVITY USING MATHEMATICAL MODELLING OF TARGET GENE EXPRESSION

(71) Applicant: InnoSIGN B.V., Eindhoven (NL)

(72) Inventors: Paul VAN SWINDEREN, Eindhoven (NL); ANJA VAN DE STOLPE, Eindhoven (NL)

(73) Assignee: InnoSIGN B.V., Eindhoven (NL)

(21) Appl. No.: 18/709,714

(22) PCT Filed: Nov. 11, 2022
(86) PCT No.: PCT/EP2022/081646

§ 371 Date: May 13, 2024

(30) Foreign Application Priority Data
Nov. 15, 2021 (EP) 21208189.7

(51) Int. Cl. G16B 25/10; C12Q 1/6851; C12Q 1/686; C12Q 1/6869; G16B 5/20; G16H 10/40; G16H 10/60; G16H 20/10; G16H 50/20

(52) U.S. Cl. CPC G16B 25/10; C12Q 1/6851; C12Q 1/686; C12Q 1/6869; G16B 5/20; G16H 10/40; G16H 10/60; G16H 20/10; G16H 50/20; C12Q 2600/158

(57) ABSTRACT

The present invention relates to a computer-implemented method for inferring activity of a GR cellular signaling pathway in a subject based on expression levels of six or more target genes of the GR cellular signaling pathway measured in a sample of the subject. The present invention further relates to an apparatus, to a non-transitory storage medium, and to a computer program for inferring activity of a GR cellular signaling pathway in a subject. The present invention further relates to a kit for measuring expression levels of six or more target genes of the GR cellular signaling pathway in a sample of a subject, to a kit for inferring activity of a GR cellular signaling pathway in a subject, and to the use of such kits in performing the method.

### CROSS-REFERENCE TO RELATED APPLICATIONS

The present application is a United States National Stage Application pursuant to 35 U.S.C. § 371 of International Patent Application No. PCT/EP2022/081646, filed on Nov. 11, 2022, which claims the benefit of EP Application No. 21208189.7, filed Nov. 15, 2021, which applications are hereby incorporated by reference in their entireties.

### FIELD OF THE INVENTION

The present invention generally relates to the field of bioinformatics, genomic processing, proteomic processing, and related arts. More particularly, the present invention relates to a computer-implemented method for inferring activity of a GR cellular signaling pathway in a subject performed by a digital processing device, wherein the inferring is based on expression levels of six or more target genes of the GR cellular signaling pathway measured in a sample of the subject. The present invention further relates to an apparatus for inferring activity of a GR cellular signaling pathway in a subject comprising a digital processor configured to perform the method, to a non-transitory storage medium for inferring activity of a GR cellular signaling pathway in a subject storing instructions that are executable by a digital processing device to perform the method, and to a computer program for inferring activity of a GR cellular signaling pathway in a subject comprising program code means for causing a digital processing device to perform the method, when the computer program is run on the digital processing device. The present invention further relates to a kit for measuring expression levels of six or more target genes of the GR cellular signaling pathway in a sample of a subject, to a kit for inferring activity of a GR cellular signaling pathway in a subject, and to uses of the kits in performing the method.

### BACKGROUND OF THE INVENTION

Genomic and proteomic analyses have substantial realized and potential promise for clinical application in medical fields such as oncology and immunology, where various cancers are known to be associated with specific combinations of genomic mutations/variations and/or high or low expression levels for specific genes, which play a role in growth and evolution of cancer, e.g., cell proliferation and metastasis.

The glucocorticoid receptor (GR, also referred to as GCR) also known as NR3C1 (nuclear receptor subfamily 3, group C, member 1) is the receptor to which cortisol and other glucocorticoids bind. The GR is expressed in almost every cell in the body and regulates genes controlling development, metabolism, and immune response. The glucocorticoid receptor (GR) is a member of the nuclear receptor family; in inactive state it is bound to Heat Shock Proteins (HSP) in the cytoplasm. Upon binding of glucocorticoid ligands (or analogs or antagonists), GR is freed from the HSPs and dimerizes to translocate to the nucleus and induce transcription of target genes that contain at least one glucocorticoid response element (GRE) in their promoter region. The final transcriptional response is also determined by the chromatic structure of the cell. The pathway plays an important role in tumor progression, metabolism, and in the immune response, and induces transcription of target genes dependent on combined epigenetic (chromatin) status of the target gene DNA, availability of active co-activator proteins and ligand activation, resulting in freeing up GR from the HSPs, enabling nuclear translocation.

Unlike other steroid hormone receptors, the glucocorticoid receptor (GR) is not considered an oncogene. In breast cancer, the estrogen receptor (ER) drives cell growth, proliferation, and metastasis, and the androgen receptor (AR) plays a similar role in prostate cancer. Accordingly, treatment of these diseases has focused on blocking steroid hormone receptor function. In contrast, glucocorticoids (GCs) work through GR to arrest growth and induce apoptosis in lymphoid tissue. Glucocorticoids are amazingly effective in this role, and have been deployed as the cornerstone of lymphoid cancer treatment for decades (Pufall M A. Glucocorticoids and Cancer. Adv Expt Med Biol 2015; 872:3 15-333. doi 10.1007/978-1-4939-2895-8_14). On the other hand GR signaling plays a role in a variety of disease such as immune related disorders. Therefore, assays that allow accurate, fast and easy determination may be a valuable tool for research and diagnostic purposes.

Current assays for determining GR pathway activity generally rely on reporter constructs with one or more GRE elements driving the expression of a reporter gene in the presence of GR dimers. By incubating the construct with nuclear extracts pathway activity can be determined. Alternatively antibodies can be used to either detect nuclear localization of the GR receptors (indicating activity) or to determine post translational modifications. Unfortunately all these methods are laborious and generally not very accurate nor quantitative, and/or not applicable to fixed tissue samples such as FFPE samples.

Assays have been described for example for determining the PR pathway activity based on the expression levels of specifically selected and validated target genes of the PR pathway in EP3822368. The GR pathway have been described in the literature, including putative target genes in for example Reddy et al. (Genome Research vol. 19, no. 12, pages 2163-2171), Chantzichristos et al. (eLife, vol. 10, 62236) or Mahita et al. (Trends in Pharmacological Sciences, vol. 34, no. 9 pages 518-530), however none of the described putative target genes have been suggested to be used for, let alone tested or validated for use in a model for determining pathway activity.

Therefore there is a need for improved, more quantitative methods. These needs, among others, are met by the invention as defined in the appended claims.

### SUMMARY OF THE INVENTION

In accordance with a main aspect of the present invention, the above problem is solved by a
computer-implemented method for inferring activity of a GR cellular signaling pathway in a sample performed by a digital processing device, wherein the inferring comprises:
   receiving expression levels of six or more target genes of the GR cellular signaling pathway measured in a sample,
   determining an activity level of a GR transcription factor (TF) element in the sample, the GR TF element controlling transcription of the six or more GR target genes, the determining being based on evaluating a calibrated mathematical pathway model relating the expression levels of the six or more GR target genes to the activity level of the GR TF element, and
   inferring the activity of the GR cellular signaling pathway in the sample based on the determined activity level of the GR TF element in the sample,
   wherein the calibrated mathematical pathway model is not germline specific and the six or more target genes are selected from the group consisting of: AKAP13, BCL6, BIRC3, DDIT4, FGD4, FKBP5, MCL1, PER1, SRGN, TSC22D3 and TSPYL2six or more.

In a second aspect the invention relates to an apparatus for inferring activity of a GR cellular signaling pathway in a sample comprising a digital processor configured to perform the method of the invention.

In a third aspect, the invention relates to a non-transitory storage medium for inferring activity of a GR cellular signaling pathway in a sample storing instructions that are executable by a digital processing device to perform the method of the invention.

In a fourth aspect the invention relates to a computer program for inferring activity of a GR cellular signaling pathway in a sample comprising program code means for causing a digital processing device to perform the method of the invention, when the computer program is run on the digital processing device.

In a fifth aspect the invention relates to a kit for measuring expression levels of six or more target genes of the GR cellular signaling pathway in a sample, comprising:
polymerase chain reaction primers directed to the six or more GR target genes, and
probes directed to the six or more GR target genes,
wherein the calibrated mathematical pathway model is not germline specific and the six or more target genes are selected from the group consisting of: AKAP13, BCL6, BIRC3, DDIT4, FGD4, FKBP5, MCL1, PER1, SRGN, TSC22D3 and TSPYL2.

In a sixth aspect the invention relates to the use of the kit according to the invention in performing the method of the invention.

In a seventh aspect the invention relates to the use of the kit according to the invention in determining the GR cellular signaling pathway activity.

### BRIEF DESCRIPTION OF THE DRAWINGS

FIG. 1 (A) shows blood model calibration on public dataset GSE11336. Glucocorticoid-sensitive T-ALL cell line CCRF-CEM-C7H2 was treated in 3 independent experiments either with 100 nM dexamethasone (a glucocorticoid) or 0.1% ethanol as an empty carrier control. Samples were taken at various timepoints (6 and 24 hours), RNA extracted and hybridized onto Affymetrix HGU133-plus2 microarrays”. (B) shows performance of the lung-calibrated model on this blood-cell dataset. Pathway activity scores are much lower and the difference between GR pathway active and inactive samples is smaller. Pathway activity score shown normalized on a scale of 0-100.

FIG. 2 (A) shows calibration epithelial model on lung calibration dataset GSE17307 “A549 cells were treated with compounds for 48 hours before RNA extraction and hybridization on Affymetrix microarrays.” (B) shows performance of the blood-calibrated model on this lung-cell dataset. Pathway activity scores are much lower for active samples and the difference between GR pathway active and inactive samples is smaller. Pathway activity score shown normalized on a scale of 0-100.

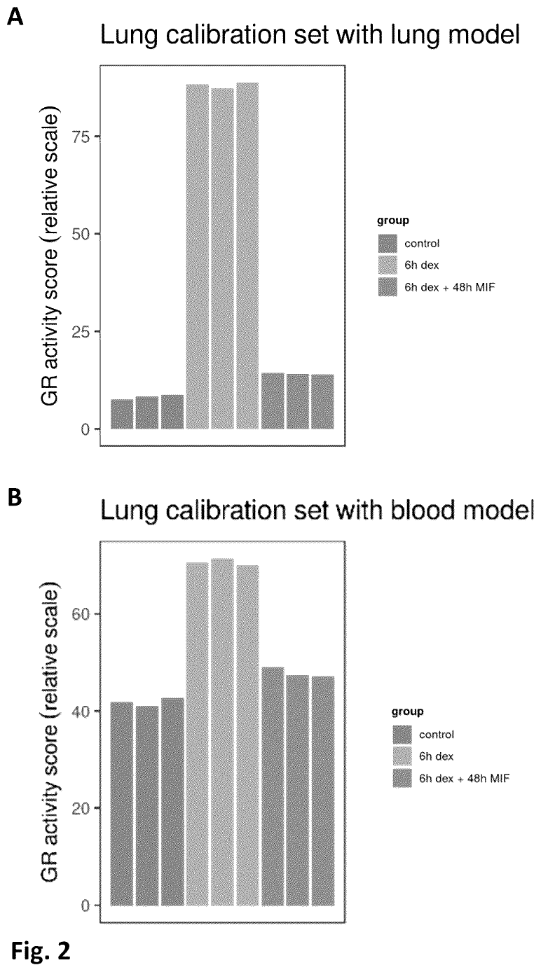

FIG. 3 shows calibration results of the generic model on the combined set of blood (GSE11336) (A) and lung (GSE17307) (B) samples. Pathway activity score shown normalized on a scale of 0-100.

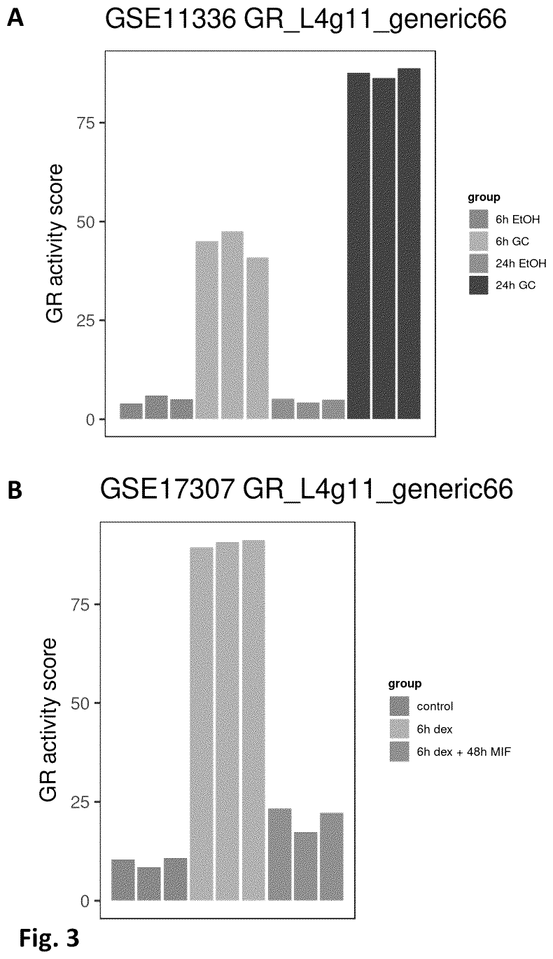

FIG. 4 shows dataset GSE2842: ALL cell lines treated with GC in vitro. Pathway activity score shown normalized on a scale of 0-100.

FIG. 5 shows dataset GSE79761: “Analysis of MCF-7 cells treated for 4 h with Ethanol, Estradiol (E2), Dexamethasone (Dex), or Estradiol+Dexamethasone (E2+Dex)”. Pathway activity score shown normalized on a scale of 0-100.

FIG. 6 shows dataset GSE22152: “Gene expression profiles of glucocorticoid (GC) resistant and sensitive T-ALL cells during GC treatment and corresponding control samples (cells treated with vehicle control).” Pathway activity score shown normalized on a scale of 0-100.

FIG. 7 shows GSE22779. Mononuclear cells (PBMCs) were isolated from peripheral blood samples before, and after 2, 6, and 24 hours of in-vivo glucocorticoid treatment of a healthy donor individual. GR pathway activity scores presented on a 0-100 scale.

FIG. 8 shows GSE30159 “Expression levels were examined in bone biopsy samples from 9 patients with endogenous Cushing syndrome, before and after a mean of three months after surgery.” Sample are matched, the nine pre-surgery samples are matched to the nine post-surgery samples, 1-1, 2-2 etc. Statistical analysis: Welch Two Sample t-test. data: pre and post; p-value=4.17E-05; Pathway activity score shown normalized on a scale of 0-100, which went down from an average 63.3 before surgery to 43.5 after surgery.

FIG. 9 shows GSE2677 “Peripheral blood lymphoblasts purified at three time points (0 h, 6-8 h, 24 h after glucocorticoid treatment initiation) from 13 children under therapy for ALL. Treated samples were compared to untreated (0 h)” Shown are measured pathway activity before and after 6 hours and 24 hours of glucocorticoid treatment, matched samples are connected through lines. The increase (delta) in GR pathway activity between 0 and 6 hrs was 10.6 points on average (SD 6.3), and between 0 and 24 hrs after start of treatment it was 13.9 on average (SD 8.6). Pathway activity score shown normalized on a scale of 0-100.

FIG. 10 shows GSE30644. MM1S cells were treated with CGS-21680 or Salmeterol alone, or in combination with dexamethasone for six hours for RNA extraction and hybridization on Affymetrix microarrays.”

FIG. 11 shows GSE113571. Triple Negative Breast cancer cell line (MDA-MB-231) treated for 4, 8, 12 hrs with vehicle, 100 nM dexamethasone, 100 nM dexamethasone and 100 nM mifrepristone (GCR antagonist), or 100 nM dexamethasone and 100 nM CORT108297 (GCR antagonist). Mifrepristone is a strong dexamethasone antagonist which is reflected in the decrease in GR pathway activity (on the y-axis). CORT108297 is minimally effective in antagonizing the effect of dexamethasone.

FIG. 12 shows an exemplary Bayesian network model used to model the transcriptional program of the GR cellular signaling pathway.

FIG. 13 shows an example flowchart illustrating a process for inferring the activity of GR cellular signaling from a sample isolated from a subject.

FIG. 14 shows an example flowchart illustrating a process for generating a calibrated mathematical pathway model.

### DETAILED DESCRIPTION OF THE INVENTION

In an embodiment, the invention relates to a
computer-implemented method for inferring activity of a GR cellular signaling pathway in a sample performed by a digital processing device, wherein the inferring comprises:
   receiving expression levels of six or more target genes of the GR cellular signaling pathway measured in a sample,
   determining an activity level of a GR transcription factor (TF) element in the sample, the GR TF element controlling transcription of the six or more GR target genes, the determining being based on evaluating a calibrated mathematical pathway model relating the expression levels of the six or more GR target genes to the activity level of the GR TF element, and
   inferring the activity of the GR cellular signaling pathway in the sample based on the determined activity level of the GR TF element in the sample,
   wherein the calibrated mathematical pathway model is not germline specific and the six or more target genes are selected from the group consisting of: AKAP13, BCL6, BIRC3, DDIT4, FGD4, FKBP5, MCL1, PER1, SRGN, TSC22D3 and TSPYL2six or more six or more six or more six or more.

In an alternative embodiment the invention also relates to a method for inferring activity of a GR cellular signaling pathway in a sample, wherein the inferring comprises:
determining the expression levels of six or more target genes of the GR cellular signaling pathway measured in a sample,
determining an activity level of a GR transcription factor (TF) element in the sample, the GR TF element controlling transcription of the six or more GR target genes, the determining being based on evaluating a calibrated mathematical pathway model relating the expression levels of the six or more GR target genes to the activity level of the GR TF element, and
inferring the activity of the GR cellular signaling pathway in the sample based on the determined activity level of the GR TF element in the sample,
wherein the calibrated mathematical pathway model is not germline specific and the six or more target genes are selected from the group consisting of: AKAP13, BCL6, BIRC3, DDIT4, FGD4, FKBP5, MCL1, PER1, SRGN, TSC22D3 and TSPYL2.

In an alternative embodiment, the invention relates to a method for inferring activity of a GR cellular signaling pathway in a sample performed by a digital processing device, wherein the inferring comprises:
determining or receiving expression levels of three or more, e.g. three, four, five, six, seven, eight, nine, ten or eleven, target genes of the GR cellular signaling pathway measured in a sample,
determining an activity level of a GR transcription factor (TF) element in the sample, the GR TF element controlling transcription of the three or more, e.g. three, four, five, six, seven, eight, nine, ten or eleven, GR target genes, the determining being based on evaluating a calibrated mathematical pathway model relating the expression levels of the three or more, e.g. three, four, five, six, seven, eight, nine, ten or eleven, GR target genes to the activity level of the GR TF element, and
inferring the activity of the GR cellular signaling pathway in the sample based on the determined activity level of the GR TF element in the sample,
wherein the calibrated mathematical pathway model is not germline specific and the three or more, e.g. three, four, five, six, seven, eight, nine, ten or eleven, target genes are selected from the group consisting of: AKAP13, BCL6, BIRC3, DDIT4, FGD4, FKBP5, MCL1, PER1, SRGN, TSC22D3 and TSPYL2,
wherein the three or more, e.g. three, four, five, six, seven, eight, nine, ten or eleven, target genes comprise one or more, e.g. one, two or three, target genes selected from AKAP13, FGD4 and TSPYL2. Optionally the method is a computer implemented method.

Herein, the “activity level” of a TF element denotes the level of activity of the TF element regarding transcription of its target genes.

The present invention is based on the innovation of the inventors that a suitable way of identifying effects occurring in the GR cellular signaling pathway can be based on a measurement of the signaling output of the GR cellular signaling pathway, which is—amongst others—the transcription of the target genes, which is controlled by a GR transcription factor (TF) element that is controlled by the GR cellular signaling pathway. This innovation by the inventors assumes that the TF activity level is at a quasi-steady state in the sample, which can be detected by means of—amongst others—the expression values of the GR target genes. The GR cellular signaling pathway targeted herein is known to control many functions in many cell types in humans, such as development, metabolism and immune response. Regarding pathological disorders, elevation in glucocorticoids has been implicated in psychiatric disorders such as schizophrenia, drug addiction, posttraumatic stress disorder (PTSD) and mood disorders. Further, glucocorticoids in treating various forms of ocular inflammation (e.g., conjunctivitis, keratitis, uveitis), macular edema, and macular degeneration. Patients with excessive levels of corticosteroids (either endogenously or exogenously) are at a higher risk of developing cardiovascular disease, while patients who developed Iatrogenic Cushing's syndrome as a result of long-term glucocorticoid treatment were at a higher risk of developing cardiovascular disease compared to the people not receiving glucocorticoid treatment. Further, glucocorticoids are a gold standard for immune suppression in organ transplant patients as they exert their classic anti-inflammatory role by acting on nearly all cell types of the immune system. Glucocorticoids, particularly the inhaled corticosteroids, are the most commonly prescribed drugs for the treatment of chronic inflammatory conditions of the respiratory tract such as asthma. A physiological appreciation for GR signaling to regulate metabolic homeostasis can be found in cases with Cushing's disease and Addison's disease, where Cushing's can result in central obesity, hyperglycemia, hypercholesterolemia, and fatty liver. In contrast, weight loss due to loss of appetite, skin discoloration, and hypoglycemia are seen in patients with Addison's disease (Kadmiel M, Cidlowski J A. Glucocorticoid receptor signaling in health and disease. Trends Pharmacol Sci. 2013; 34(9):518-530). Thus, the abnormal GR cellular signaling activity plays an important role, which is detectable in the expression profiles of the target genes and thus exploited by means of a calibrated mathematical pathway model.

The present invention makes it possible to determine the activity of the GR cellular signaling pathway in a sample by (i) determining an activity level of a GR TF element in the sample, wherein the determining is based on evaluating a calibrated mathematical model relating the expression levels of six or more target genes of the GR cellular signaling pathway, the transcription of which is controlled by the GR TF element, to the activity level of the GR TF element, and by (ii) inferring the activity of the GR cellular signaling pathway in the sample based on the determined activity level of the GR TF element in the sample. This preferably allows improving the possibilities of characterizing patients that have a disease, such as an immune disorder, metabolic disease or cancer, which is at least partially driven by an abnormal activity of the GR cellular signaling pathway, and that are therefore likely to respond to inhibitors of the GR cellular signaling pathway. Likewise, in cases where the GR cellular signaling pathway is acting as a protective (e.g. as tumor suppressive) pathway, an abormally low activity of the pathway may preferably be identified using the methods of the present invention and a treatment that increases the activity of the GR cellular signaling pathway, for instance, by providing glucocorticoids to the patient, may be given to the patient. In particular embodiments, treatment determination can be based on a specific GR cellular signaling pathway activity. In a particular embodiment, the GR cellular signaling status can be set at a cutoff value of odds of the GR cellular signaling pathway being active of, for example, 10:1, 5:1, 4:1, 2:1, 1:1, 1:2, 1:4, 1:5, or 1:10.

One particular issue that is overcome by the present invention, is that the model can be used regardless of the embryonic origin (germ layer) of tissue or cell type it is used on. It was found by the inventors that the target genes of the GR receptor signaling pathway may deviate to some extent in different cell types. It was found that there was a significant correlation between the target genes and the embryonic origin of the cell or tissue type. For example some target genes that are strongly regulated in blood cells (mesodermal origin) are barely regulated by GR signaling in lung cells (endodermal origin), and vice versa. Some genes were even found to have opposite regulation between the different germ layers (e.g. upregulated by GR signaling in blood and downregulated by GR signaling in lung cells). This has prompted the inventors to develop a model which can be applied to all germ layers. The presently selected target genes were surprisingly found to be uniformly regulated by GR signaling in mesoderm cells and endoderm/ectoderm cells.

Glucocorticoids (also referred to as glucocorticosteroids) are a class of corticosteroids, which are a class of steroid hormones. Glucocorticoids are corticosteroids that bind to the glucocorticoid receptor. Glucocorticoids affect cells by binding to the glucocorticoid receptor. In the absence of hormone, the glucocorticoid receptor (GR) resides in the cytosol complexed with a variety of proteins including heat shock protein 90 (hsp90), the heat shock protein 70 (hsp70) and the protein FKBP4 (FK506-binding protein 4). The endogenous glucocorticoid hormone cortisol, or any other glucocorticoid, diffuses through the cell membrane into the cytoplasm and binds to the glucocorticoid receptor (GR) resulting in release of the heat shock proteins. The resulting activated form GR has two principal mechanisms of action, transactivation and transrepression. In transactivation a direct mechanism of action involves homodimerization of the receptor, translocation via active transport into the nucleus, and binding to specific DNA responsive elements activating gene transcription. The biological response depends on the cell type. In trans repression the absence of activated GR, other transcription factors such as NF-κB or AP-1 themselves are able to transactivate target genes. However activated GR can complex with these other transcription factors and prevent them from binding their target genes and hence repress the expression of genes that are normally upregulated by NF-κB or AP-1.

Herein, the term “Glucocorticoid receptor transcription factor element” or “GR TF element” or “TF element” is defined to be a protein complex containing at least a nuclear Glucocorticoid receptor, optionally with co-factors. Preferably, the term refers to either a protein or protein complex transcriptional factor, activated by binding a specific ligand such as glucocorticoid or by an activating mutation in the GR gene. The GR TF element therefore preferably refers to either one of a GR-alpha or GR-beta monomer, a homodimer of GR-alpha, a homodimer of GR-beta, a GR-alpha-GR-beta heterodimer. Preferably the GR TF element binds a glucocorticoid or is mutated to allow activity in the absence of binding glucocorticoid. Optionally the GR TF element is further bound to a co-factor which may act as a transcription factor. Exemplary but non-limiting examples of co-factors are STAT3, STAT5, SMAD3, SMAD4, SMAD6, AP-1, NF-κB, DAXX, p160 and GRIP1.

The most common glucocorticoid is cortisol, however many glucocorticoids are known. Glucocorticoids can be divided in natural and synthetic glucocorticoids. Therefore when used herein a glucocorticoid may refer to either a natural or a synthetic glucocorticoid.

Non-limiting examples of natural glucocorticoids are 11-Dehydrocorticosterone (11-oxocorticosterone, 17-deoxycortisone)=21-hydroxypregn-4-ene-3,11,20-trione, 11-Deoxycorticosterone (deoxycortone, desoxycortone; 21-hydroxyprogesterone)=21-hydroxypregn-4-ene-3,20-dione, 11-Deoxycortisol (cortodoxone, cortexolone)=17α,21-dihydroxypregn-4-ene-3,20-dione, 11-Ketoprogesterone (11-oxoprogesterone; Ketogestin)=pregn-4-ene-3,11,20-trione, 11β-Hydroxypregnenolone=3β,11β-dihydroxypregn-5-en-20-one, 11β-Hydroxyprogesterone (21-deoxycorticosterone)=11β-hydroxypregn-4-ene-3,20-dione, 11β,17α,21-Trihydroxypregnenolone=3β,11β,17α,21-tetrahydroxypregn-5-en-20-one, 17α,21-Dihydroxypregnenolone=3β,17α,21-trihydroxypregn-5-en-20-one, 17α-Hydroxypregnenolone=3β,17α-dihydroxypregn-5-en-20-one, 17α-Hydroxyprogesterone=17α-hydroxypregn-4-ene-3,11,20-trione, 18-Hydroxy-11-deoxycorticosterone=18,21-dihydroxypregn-4-ene-3,20-dione, 18-Hydroxycorticosterone=11β,18,21-trihydroxypregn-4-ene-3,20-dione, 18-Hydroxyprogesterone=18-hydroxypregn-4-ene-3,20-dione, 21-Deoxycortisol=11β,17α-dihydroxypregn-4-ene-3,20-dione, 21-Deoxycortisone=17α-hydroxypregn-4-ene-3,11,20-trione, 21-Hydroxypregnenolone (prebediolone)=3β,21-dihydroxypregn-5-en-20-one, Aldosterone=11β,21-dihydroxypregn-4-ene-3,18,20-trione, Corticosterone (17-deoxycortisol)=11β,21-dihydroxypregn-4-ene-3,20-dione, Cortisol (hydrocortisone)=11β,17α,21-trihydroxypregn-4-ene-3,20-dione Cortisone=17α,21-dihydroxypregn-4-ene-3,11,20-trione, Pregnenolone=pregn-5-en-3β-ol-20-one and Progesterone=pregn-4-ene-3,20-dione.

Non-limiting examples of synthetic glucocorticoids are Flugestone (flurogestone)=9α-fluoro-11β,17α-dihydroxypregn-4-ene-3,20-dione, Fluorometholone=6α-methyl-9α-fluoro-11β,17α-dihydroxypregna-1,4-diene-3,20-dione, Medrysone (hydroxymethylprogesterone)=6α-methyl-11β-hydroxypregn-4-ene-3,20-dione, rebediolone acetate (21-acetoxypregnenolone)=3β,21-dihydroxypregn-5-en-20-one 21-acetate, Chloroprednisone=6α-chloro-17α,21-dihydroxypregna-1,4-diene-3,11,20-trione, Cloprednol=6-chloro-11β,17α,21-trihydroxypregna-1,4,6-triene-3,20-dione, Difluprednate=6α,9α-difluoro-11β,17α,21-trihydroxypregna-1,4-diene-3,20-dione 17α-butyrate 21-acetate, Fludrocortisone=9α-fluoro-11β,17α,21-trihydroxypregn-4-ene-3,20-dione, Fluocinolone=6α,9α-difluoro-11β,16α,17α,21-tetrahydroxypregna-1,4-diene-3,20-dione, Fluperolone=9α-fluoro-11β,17α,21-trihydroxy-21-methylpregna-1,4-diene-3,20-dione, Fluprednisolone=6α-fluoro-11β,17α,21-trihydroxypregna-1,4-diene-3,20-dione, Loteprednol=11β,17α,dihydroxy-21-oxa-21-chloromethylpregna-1,4-diene-3,20-dione, Methylprednisolone=6α-methyl-11β,17α,21-trihydroxypregna-1,4-diene-3,20-dione, Prednicarbate=11β,17α,21-trihydroxypregna-1,4-diene-3,20-dione 17α-ethylcarbonate 21-propionate, Prednisolone=11β,17α,21-trihydroxypregna-1,4-diene-3,20-dione, Prednisone=17α,21-dihydroxypregna-1,4-diene-3,11,20-trione, Tixocortol=11β,17α-dihydroxy-21-sulfanylpregn-4-ene-3,20-dione, Triamcinolone=9α-fluoro-11β,16α,17α,21-tetrahydroxypregna-1,4-diene-3,20-dione, Alclometasone=7α-chloro-11β,17α,21-trihydroxy-16α-methylpregna-1,4-diene-3,20-dione, Beclometasone=9α-chloro-11β,17α,21-trihydroxy-16β-methylpregna-1,4-diene-3,20-dione, Betamethasone=9α-fluoro-11β,17α,21-trihydroxy-16β-methylpregna-1,4-diene-3,20-dione, Clobetasol=9α-fluoro-11β,17α-dihydroxy-16β-methyl-21-chloropregna-1,4-diene-3,20-dione, Clobetasone=9α-fluoro-16β-methyl-17α-hydroxy-21-chloropregna-1,4-diene-3,11,20-trione, Clocortolone=6α-fluoro-9α-chloro-11β,21-dihydroxy-16α-methylpregna-1,4-diene-3,20-dione, Desoximetasone=9α-fluoro-11β,21-dihydroxy-16α-methylpregna-1,4-diene-3,20-dione, Dexamethasone=9α-fluoro-11β,17α,21-trihydroxy-16α-methylpregna-1,4-diene-3,20-dione, Diflorasone=6α,9α-difluoro-11β,17α,21-trihydroxy-16β-methylpregna-1,4-diene-3,20-dione, Difluocortolone=6α,9α-difluoro-11β,21-dihydroxy-16α-methylpregna-1,4-diene-3,20-dione, Fluclorolone=6α-fluoro-9α,11β-dichloro-16α,17α,21-trihydroxypregna-1,4-dien-3,20-dione, Flumetasone=6α,9α-difluoro-11β,17α,21-trihydroxy-16α-methylpregna-1,4-diene-3,20-dione, Fluocortin=6α-fluoro-11β,21-dihydroxy-16α-methylpregna-1,4-diene-3,20,21-trione, Fluocortolone=6α-fluoro-11β,21-dihydroxy-16α-methylpregna-1,4-diene-3,20-dione, Fluprednidene=9α-fluoro-11β,17α,21-trihydroxy-16-methylenepregna-1,4-diene-3,20-dione, Fluticasone=6α,9α-difluoro-11β,17α-dihydroxy-16α-methyl-21-thia-21-fluoromethylpregna-1,4-dien-3,20-dione, Fluticasone furoate=6α,9α-difluoro-11β,17α-dihydroxy-16α-methyl-21-thia-21-fluoromethylpregna-1,4-dien-3,20-dione 17α-(2-furoate), Halometasone=2-chloro-6α,9α-difluoro-11β,17α,21-trihydroxy-16α-methylpregna-1,4-diene-3,20-dione, Meprednisone=16β-methyl-17α,21-dihydroxypregna-1,4-diene-3,11,20-trione,Mometasone=9α,21-dichloro-11β,17α-dihydroxy-16α-methylpregna-1,4-diene-3,20-dione, Mometasone furoate=9α,21-dichloro-11β,17α-dihydroxy-16α-methylpregna-1,4-diene-3,20-dione 17α-(2-furoate) Paramethasone=6α-fluoro-11β,17α,21-trihydroxy-16α-methylpregna-1,4-diene-3,20-dione, Prednylidene=11β,17α,21-trihydroxy-16-methylenepregna-1,4-diene-3,20-dione, Rimexolone=11β-hydroxy-16α,17α,21-trimethylpregna-1,4-dien-3,20-dione, Ulobetasol (halobetasol)=6α,9α-difluoro-11β,17α-dihydroxy-16β-methyl-21-chloropregna-1,4-diene-3,20-dione, Amcinonide=9α-fluoro-11β,16α,17α,21-tetrahydroxypregna-1,4-diene-3,20-dione cyclic 16α,17α-acetal with cyclopentanone, 21-acetate, Budesonide=11β,16α,17α,21-tetrahydroxypregna-1,4-diene-3,20-dione cyclic 16α,17α-acetal with butyraldehyde, Ciclesonide=11β,16α,17α,21-tetrahydroxypregna-1,4-diene-3,20-dione cyclic 16α,17α-acetal with (R)-cyclohexanecarboxaldehyde, 21-isobutyrate, Deflazacort=11β,21-dihydroxy-2′-methyl-5′H-pregna-1,4-dieno[17,16-d]oxazole-3,20-dione 21-acetate, Desonide=11β,16α,17α,21-tetrahydroxypregna-1,4-diene-3,20-dione cyclic 16α,17α-acetal with acetone, Formocortal (fluoroformylone)=3-(2-chloroethoxy)-9α-fluoro-11β,16α,17α,21-tetrahydroxy-20-oxopregna-3,5-diene-6-carboxaldehyde cyclic 16α,17α-acetal with acetone, 21-acetate, Fluclorolone acetonide (flucloronide)=6α-fluoro-9α,11β-dichloro-16α,17α,21-trihydroxypregna-1,4-dien-3,20-dione cyclic 16α,17α-acetal with acetone, Fludroxycortide (flurandrenolone, flurandrenolide)=6α-fluoro-11β,16α,17α,21-tetrahydroxypregn-4-ene-3,20-dione cyclic 16α,17α-acetal with acetone, Flunisolide=6α-fluoro-11β,16α,17α,21-tetrahydroxypregna-1,4-diene-3,20-dione cyclic 16α,17α-acetal with acetone, Fluocinolone acetonide=6α,9α-difluoro-11β,16α,17α,21-tetrahydroxypregna-1,4-diene-3,20-dione cyclic 16α,17α-acetal with acetone, Fluocinonide=6α,9α-difluoro-11β,16α,17α,21-tetrahydroxypregna-1,4-diene-3,20-dione cyclic 16α,17α-acetal with acetone, 21-acetate, Halcinonide=9α-fluoro-11β,16α,17α-trihydroxy-21-chloropregn-4-ene-3,20-dione cyclic 16α,17α-acetal with acetone, Triamcinolone acetonide=9α-fluoro-11β,16α,17α,21-tetrahydroxypregna-1,4-diene-3,20-dione cyclic 16α,17α-acetal with acetone, Cortivazol=6,16α-dimethyl-11β,17α,21-trihydroxy-2′-phenyl[3,2-c]pyrazolopregna-4,6-dien-20-one 21-acetate, and RU-28362=6-methyl-11β,17β-dihydroxy-17α-(1-propynyl)androsta-1,4,6-trien-3-one.

The calibrated mathematical pathway model may be a probabilistic model, preferably a Bayesian network model, based on conditional probabilities relating the activity level of the GR TF element and the expression levels of the six or more GR target genes, or the calibrated mathematical pathway model may be based on one or more linear combination(s) of the expression levels of the six or more GR target genes. In particular, the inferring of the activity of the GR cellular signaling pathway may be performed as disclosed in the published international patent application WO 2013/011479 A2 (“Assessment of cellular signaling pathway activity using probabilistic modeling of target gene expression”) or as described in the published international patent application WO 2014/102668 A2 (“Assessment of cellular signaling pathway activity using linear combination(s) of target gene expressions”), the contents of which are herewith incorporated in their entirety. Further details regarding the inferring of cellular signaling pathway activity using mathematical modeling of target gene expression can be found in Verhaegh W. et al., “Selection of personalized patient therapy through the use of knowledge-based computational models that identify tumor-driving signal transduction pathways”, Cancer Research, Vol. 74, No. 11, 2014, pages 2936-2945.

The term “sample”, as used herein, refers to a specimen containing cells. The sample may be obtained from a tissue, a cell culture or a subject. The sample may be treated such as fixation by formalin. The term sample may also refer to a lysate of cells or its purified components, such as isolated RNA. When obtained from a subject the sample may for example be a biopsy or a blood sample.

The term “subject”, as used herein, refers to any living being. In some embodiments, the subject is an animal, preferably a mammal. In certain embodiments, the subject is a human being, preferably a medical subject.

The term “target gene” as used herein, means a gene whose transcription is directly or indirectly controlled by a GR transcription factor element. The “target gene” may be a “direct target gene” and/or an “indirect target gene” (as described herein).

Particularly suitable GR target genes are described in the following text passages as well as the examples below.

In an embodiment the sample is obtained from a subject. In an embodiment the method for inferring activity of a GR cellular signaling pathway in a sample further comprises the step of obtaining the sample from the subject.

Another aspect of the present invention relates to a method (as described herein), further comprising:
determining whether the GR cellular signaling pathway is operating at an undesired activity level in the sample based on the inferred activity of the GR cellular signaling pathway in the sample.

Another aspect of the present invention relates to a method (as described herein), further comprising:
recommending prescribing a drug for the subject that corrects for undesired activity level operation of the GR cellular signaling pathway,
wherein the recommending is performed if the GR cellular signaling pathway is determined to be operating at an undesired activity level in the sample based on the inferred activity of the GR cellular signaling pathway.

The phrase “the cellular signaling pathway is operating abnormally” refers to the case where the “activity” of the pathway is not as expected, wherein the term “activity” may refer to the activity of the transcription factor complex in driving the target genes to expression, i.e., the speed by which the target genes are transcribed. “Normal” may be when it is inactive in tissue where it is expected to be inactive and active where it is expected to be active. Furthermore, there may be a certain level of activity that is considered “normal”, and anything higher or lower maybe considered “abnormal”.

The present invention also relates to a method (as described herein), wherein the abnormal operation of the GR cellular signaling pathway is an operation in which the GR cellular signaling pathway operates as a tumor suppressor, an immune suppressor, or a driver of a disease or a disorder.

The sample(s) to be used in accordance with the present invention can be an extracted sample, that is, a sample that has been extracted from the subject. Examples of the sample include, but are not limited to, a tissue, cells, blood and/or a body fluid of a subject. If the subject is a medical subject that has or may have cancer, it can be, e.g., a sample obtained from a cancer lesion, or from a lesion suspected for cancer, or from a metastatic tumor, or from a body cavity in which fluid is present which is contaminated with cancer cells (e.g., pleural or abdominal cavity or bladder cavity), or from other body fluids containing cancer cells, and so forth, preferably via a biopsy procedure or other sample extraction procedure. The cells of which a sample is extracted may also be tumorous cells from hematologic malignancies (such as leukemia or lymphoma). In some cases, the cell sample may also be circulating tumor cells, that is, tumor cells that have entered the bloodstream and may be extracted using suitable isolation techniques, e.g., apheresis or conventional venous blood withdrawal. If the subject is a medical subject that has or may have a different disease (meaning not cancer) the sample may be a sample obtained from a tissue relevant for the disease or representative for the disease. Aside from blood, a body fluid of which a sample is extracted may be urine, gastrointestinal contents, or an extravasate. The term “sample”, as used herein, also encompasses the case where e.g. a tissue and/or cells and/or a body fluid of the subject have been taken from the subject and, e.g., have been put on a microscope slide, and where for performing the claimed method a portion of this sample is extracted, e.g., by means of Laser Capture Microdissection (LCM), or by scraping off the cells of interest from the slide, or by fluorescence-activated cell sorting techniques. In addition, the term “sample”, as used herein, also encompasses the case where e.g. a tissue and/or cells and/or a body fluid of the subject have been taken from the subject and have been put on a microscope slide, and the claimed method is performed on the slide. In addition, the term “sample”, as used herein, also encompasses the case where e.g. a cell line and/or cell culture has been generated based on the cells/tissue/body fluid that have been taken from the subject.

Another aspect of the present invention relates to a method (as described herein), wherein the undesired activity level in which the GR cellular signaling pathway operates as a promoter of, or fails to inhibit, or is an indication of one or more of the following disorders: cancer, a psychiatric disorder, an eye disorder, cardiovascular disease, inflammatory and immune related disorders, a respiratory disorder, a musculoskeletal disorders, a cutaneous inflammatory condition, or a metabolic disorder.

In an aspect the cancer is leukemia, childhood leukemia, B-cell acute lymphoblastic leukemia, T-cell acute lymphoblastic leukemia, lymphoid cancer, adult acute lymphoblastic leukemia (ALL), multiple myeloma (MM), Hodgkin's disease, Chronic lymphocytic leukemia (CLL), non-Hodgkin's lymphomas (NHL) or a solid tumor such as prostate cancer or Kaposi sarcoma (Pufall MA. Glucocorticoids and Cancer. Adv Exp Med Biol. 2015; 872:315-333, incorporated by reference in its entirety). The GR pathway is generally considered a tumor suppressing pathway, therefore GR pathway activity is in general relevant in cancer as: 1) glucocorticoids are often administered as co-treatment, abnormal GR pathway activity can predict its effectivity for a patient. 2) Abnormally low GR pathway activity may contribute to tumor growth due to the lack of GR tumor suppressing function. However in some cases GR pathway activity may be oncogenic, for example in breast or prostate cancer. Therefore in general in cancer patients an undesired activity level of the GR signaling pathway is a low activity level which is preferably corrected by recommending prescribing or by administering a treatment that increases GR pathway activity, however in breast or prostate cancer patients an undesired activity level of the GR signaling pathway is a high activity level which is preferably corrected by recommending prescribing or by administering a treatment that decreases GR pathway activity.

In an aspect the psychiatric disorder is selected from schizophrenia, drug addiction, posttraumatic stress disorder (PTSD) or mood disorders. Such disorders are associated with elevated levels of glucocorticoids and subsequent GR pathway signaling. Therefore in psychiatric disorder patients an undesired activity level of the GR signaling pathway is a high activity level which is preferably corrected by recommending prescribing or by administering a treatment that decrease GR pathway activity.

In an aspect the eye disorder is selected from ocular inflammation (e.g., conjunctivitis, keratitis, uveitis), macular edema, macular degeneration or glaucoma. Glucocorticoids are often prescribed in these disorders for their anti-inflammatory and anti-angiogenic functions. Therefore in eye disorder patients an undesired activity level of the GR signaling pathway is a low activity level which is preferably corrected by recommending prescribing or by administering a treatment that increases GR pathway activity.

In an aspect the disorder is cardiovascular disease. Patients with excessive levels of corticosteroids (either endogenously or exogenously) are at a higher risk of developing cardiovascular disease. Therefore in cardiovascular disease patients an undesired activity level of the GR signaling pathway is a high activity level which is preferably corrected by recommending prescribing or by administering a treatment that decrease GR pathway activity.

In an aspect the immune disorder is selected from organs transplantation, inflammation, an autoimmune disorder or a B cell malignancy. Glucocorticoids are often administered for their immune suppressive functions. Therefore in immune disorder patients an undesired activity level of the GR signaling pathway is a low activity level which is preferably corrected by recommending prescribing or by administering a treatment that increases GR pathway activity.

In an aspect the respiratory disorder is a chronic inflammatory conditions of the respiratory tract such as asthma or COPD. For respiratory disorders glucocorticoids are often prescribed to suppress cytokines and chemokines. Therefore in respiratory disorder patients an undesired activity level of the GR signaling pathway is a low activity level which is preferably corrected by recommending prescribing or by administering a treatment that increases GR pathway activity.

In an aspect the musculoskeletal disorders is rheumatoid arthritis (RA) or osteoporosis. For RA patient glucocorticoids may be prescribed for their immune suppressing function. Therefore in RA patients an undesired activity level of the GR signaling pathway is a low activity level which is preferably corrected by recommending prescribing or by administering a treatment that increases GR pathway activity. Osteoporosis may be caused by long term exposure to high glucocorticoids exposure. Therefore in osteoporosis patients an undesired activity level of the GR signaling pathway is a high activity level which is preferably corrected by recommending prescribing or by administering a treatment that decrease GR pathway activity.

In an aspect the cutaneous inflammatory condition is eczema or psoriasis. Topical corticosteroids are commonly used for treating cutaneous inflammatory conditions, such as eczema and psoriasis, due to their anti-proliferative and anti-inflammatory action. Therefore in patients with a cutaneous inflammatory condition an undesired activity level of the GR signaling pathway is a low activity level which is preferably corrected by recommending prescribing or by administering a treatment that increases GR pathway activity. Osteoporosis may be caused by long term exposure to high glucocorticoids exposure.

In an aspect the metabolic disorder is Cushing's disease, Addison's disease, obesity or hyperglycemia. In patients suffering from Cushing's disease, obesity or hyperglycemia excessive glucocorticoids are found. Therefore in patients suffering from Cushing's disease, obesity or hyperglycemia an undesired activity level of the GR signaling pathway is a high activity level which is preferably corrected by recommending prescribing or by administering a treatment that decrease GR pathway activity. Therefore in patients with a Addison's disease an undesired activity level of the GR signaling pathway is a low activity level which is preferably corrected by recommending prescribing or by administering a treatment that increases GR pathway activity. Osteoporosis may be caused by long term exposure to high glucocorticoids exposure.

As can be understood from the above passages, the methods described herein may be used to determine GR pathway activity in a sample from a subject, which pathway activity may further be used to determine a treatment strategy. The following two situations can be discerned:
1) the GR pathway has aberrant activity (e.g. the activity is higher or lower than expected based on previously determined activities in related or comparable samples). In such case the aberrant activity of the GR pathway is the undesired activity level, and the treatment strategy may be chosen such as to correct the pathway activity to a more normal (desired) level. E.g. if high GR pathway activity is measured in the blood of a subject this may indicate a risk of developing cardiovascular disease, in which case the treatment strategy may comprises administering an inhibitor of the GR pathway.
2) stimulation or inhibition of the GR pathway is intended as part of a treatment regimen, in which case normal GR pathway activity is the undesired activity level and the treatment regimen comprises stimulating or inhibiting the GR pathway activity. For example, in many tumors glucocorticoids are administered. In the tumor, low GR pathway activity may be normal for the tissue type but undesirable as the GR pathway has a tumor suppressing effect, in which case the treatment strategy may comprises administering a glucocorticoid (GR pathway activator).

Therefore, in an embodiment the method further comprises recommending prescribing a drug. The method may also comprises the method of administering a drug in a subject in need thereof. The treatment strategy may be to treat an existing condition or to prevent a condition based on an elevated risk. The treatment strategy may comprise administering an activator or an inhibitor of the GR pathway to correct the undesired activity level. An activator of the GR pathway may be a natural or a synthetic glucocorticoid as described herein above. An inhibitor of the GR pathway may be RTI 3021-012, RTI 3021-022, Cyproterone acetate, RU-486, RU-43044, 11-Monoaryl steroids, 11,21 Bisaryl Steroids, 11β-Substituted Steroids, Bridged 11β-Aryl Steroids, 10β-Substituted Steroids, Org 34517, Org 36410, Org 34850, 11β-Aryl Conjugates of Mifepristone, SAR for 11β-Aryl Substituents in Mifepristone, 11β-Aryl Substituted Mifepristone Derivatives, A-348441, Steroidal Phosphonates, Phosphorus-Containing Mifepristone Analogs, Octahydrophenanthrenes, CP-409069, CP-394531, Sprirocyclic Dihydropyridines, CP-47255, Triphenylmethanes and Diaryl ethers, KB285, AL-438, Chromenes, Dibenzyl anilines, Azadecalins, Aryl Pyrazolo Azadecalins, 8a-Benzyl Isoquinolones, Fluorocortivazol, or combinations thereof (as described in Clark R D. Glucocorticoid receptor antagonists. Curr Top Med Chem. 2008; 8(9):813-38. hereby incorporated by reference in its entirety).

Therefore, in an embodiment the invention relates to a GR cellular signaling pathway agonist or a GR cellular signaling pathway antagonist for use in the treatment, prevention or amelioration of a disease, the use comprising inferring activity of the GR cellular pathway as broadly described herein in a sample obtained from a subject, determining if the GR cellular signaling pathway activity in the sample is aberrant, and if the pathway activity is aberrant administering the GR cellular signaling pathway agonist or antagonist to the subject. Alternatively the invention relates to a method of treating, preventing or ameliorating a disease in a subject in need thereof, the method comprising, inferring activity of the GR cellular pathway as broadly described herein in a sample obtained from a subject, determining if the GR cellular signaling pathway activity in the sample is aberrant, and if the pathway activity is aberrant administering the GR cellular signaling pathway agonist or antagonist to the subject. In an embodiment the disease is selected from cancer, a psychiatric disorder, an eye disorder, cardiovascular disease, inflammatory and immune related disorders, a respiratory disorder, a musculoskeletal disorders, a cutaneous inflammatory condition, or a metabolic disorder. In an embodiment the agonist of the GR cellular signaling pathway is a natural or a synthetic glucocorticoid as described herein above. In an embodiment the antagonist of the GR cellular signaling pathway is RTI 3021-012, RTI 3021-022, Cyproterone acetate, RU-486, RU-43044, 11-Monoaryl steroids, 11,21 Bisaryl Steroids, 11β-Substituted Steroids, Bridged 11β-Aryl Steroids, 10β-Substituted Steroids, Org 34517, Org 36410, Org 34850, 11β-Aryl Conjugates of Mifepristone, SAR for 11β-Aryl Substituents in Mifepristone, 11β-Aryl Substituted Mifepristone Derivatives, A-348441, Steroidal Phosphonates, Phosphorus-Containing Mifepristone Analogs, Octahydrophenanthrenes, CP-409069, CP-394531, Sprirocyclic Dihydropyridines, CP-47255, Triphenylmethanes and Diaryl ethers, KB285, AL-438, Chromenes, Dibenzyl anilines, Azadecalins, Aryl Pyrazolo Azadecalins, 8a-Benzyl Isoquinolones, Fluorocortivazol, or combinations thereof.

In an embodiment the method of the invention is used in at least one of the following activities:
diagnosis based on the inferred activity of the GR cellular signaling pathway in the sample;
prognosis based on the inferred activity of the GR cellular signaling pathway in the sample;
drug prescription based on the inferred activity of the GR cellular signaling pathway in the sample;
prediction of drug efficacy based on the inferred activity of the GR cellular signaling pathway in the sample;
prediction of adverse effects based on the inferred activity of the GR cellular signaling pathway in the sample;
monitoring of drug efficacy;
drug development;
assay development;
pathway research;
enrollment of the subject in a clinical trial based on the inferred activity of the GR cellular signaling pathway in a sample obtained from the subject;
selection of subsequent test to be performed;
selection of companion diagnostic tests;
assessment of GR pathway activity in a sample of cells cultured or kept in vitro, for example cell lines, organoids, organ-on-chip; and
for research such as stem cell research.

In an embodiment in the method of the invention the calibrated mathematical pathway model is a probabilistic model, preferably a Bayesian network model, based on conditional probabilities relating the activity level of the GR TF element and the expression levels of the six or more GR target genes, or wherein the mathematical pathway model is based on one or more (pseudo-)linear combination(s) of the expression levels of the six or more GR target genes.

In an embodiment the method of the invention further comprises determining the expression levels of the six or more GR target genes in the sample, preferably wherein said determining the expression levels of the six or more GR target genes in the sample is based on mRNA extracted from the sample.

In accordance with another disclosed aspect, an apparatus for inferring activity of a GR cellular signaling pathway in a sample comprises a digital processor configured to perform the method of the present invention as described herein.

In accordance with another disclosed aspect, a non-transitory storage medium for inferring activity of a GR cellular signaling pathway in a sample, stores instructions that are executable by a digital processing device to perform the method of the present invention as described herein. The non-transitory storage medium may be a computer-readable storage medium, such as a hard drive or other magnetic storage medium, an optical disk or other optical storage medium, a random access memory (RAM), read only memory (ROM), flash memory, or other electronic storage medium, a network server, or so forth. The digital processing device may be a handheld device (e.g., a personal data assistant or smartphone), a notebook computer, a desktop computer, a tablet computer or device, a remote network server, or so forth.

In accordance with another disclosed aspect, a computer program for inferring activity of a GR cellular signaling pathway in a sample comprises program code means for causing a digital processing device to perform the method of the present invention as described herein, when the computer program is run on the digital processing device. The digital processing device may be a handheld device (e.g., a personal data assistant or smartphone), a notebook computer, a desktop computer, a tablet computer or device, a remote network server, or so forth.

In accordance with another disclosed aspect, a kit for measuring expression levels of six or more target genes of the GR cellular signaling pathway in a sample, comprising:
polymerase chain reaction primers directed to the six or more GR target genes, and
probes directed to the six or more GR target genes,
wherein the six or more, e.g. three, four, five, six, seven, eight, nine, ten or eleven target genes are selected from the group consisting of: AKAP13, BCL6, BIRC3, DDIT4, FGD4, FKBP5, MCL1, PER1, SRGN, TSC22D3 and TSPYL2. Alternatively the invention may relate to a kit comprising components or means for measuring the expression levels of six or more target genes of the GR cellular signaling pathway, wherein the six or more, e.g. three, four, five, six, seven, eight, nine, ten or eleven target genes are selected from the group consisting of: AKAP13, BCL6, BIRC3, DDIT4, FGD4, FKBP5, MCL1, PER1, SRGN, TSC22D3 and TSPYL2.

The one or more components or means for measuring the expression levels of the six or more GR target genes can be selected from the group consisting of: a DNA array chip, an oligonucleotide array chip, a protein array chip, an antibody, a plurality of probes, for example, labeled probes, a set of RNA reverse-transcriptase sequencing components, and/or RNA or DNA, including cDNA, amplification primers. In an embodiment, the kit includes a set of labeled probes directed to a portion of an mRNA or cDNA sequence of the six or more GR target genes as described herein. In an embodiment, the kit includes a set of primers and probes directed to a portion of an mRNA or cDNA sequence of the six or more GR target genes. In an embodiment, the labeled probes are contained in a standardized 96-well plate. In an embodiment, the kit further includes primers or probes directed to a set of reference genes. Such reference genes can be, for example, constitutively expressed genes useful in normalizing or standardizing expression levels of the target gene expression levels described herein.

In accordance with another disclosed aspect, a kit for inferring activity of a GR cellular signaling pathway in a sample comprises:
the kit of the present invention as described herein, and
the apparatus of the present invention as described herein, the non-transitory storage medium of the present invention as described herein, or the computer program of the present invention as described herein.

In accordance with another disclosed aspect, the kits of the present invention as described herein are used in performing the method of the present invention as described herein.

In accordance with another disclosed aspect, the use of the kits of the present invention as described herein in performing the method of the present invention as described herein.

In accordance with another disclosed aspect, the use of the kits of the present invention as described herein in determining the GR pathway activity.

The present invention as described herein can, e.g., also advantageously be used in at least one of the following activities:
diagnosis based on the inferred activity of the GR cellular signaling pathway in the sample;
prognosis based on the inferred activity of the GR cellular signaling pathway in the sample;
drug prescription based on the inferred activity of the GR cellular signaling pathway in the sample;
prediction of drug efficacy based on the inferred activity of the GR cellular signaling pathway in the sample;
prediction of adverse effects based on the inferred activity of the GR cellular signaling pathway in the sample;
monitoring of drug efficacy;
drug development;
assay development;
pathway research;
enrollment of the subject in a clinical trial based on the inferred activity of the GR cellular signaling pathway in a sample obtained from the subject;
selection of subsequent test to be performed;
selection of companion diagnostic tests;
assessment of GR pathway activity in a sample of cells cultured or kept in vitro, for example cell lines, organoids, organ-on-chip; and
for research such as stem cell research.

In alternative embodiments the methods, products and uses described herein are based on three or more, e.g. three, four, five, six, seven, eight, nine, ten or eleven, target genes of the GR cellular signaling pathway, wherein the three or more, e.g. three, four, five, six, seven, eight, nine, ten or eleven, target genes are selected from the group consisting of: AKAP13, BCL6, BIRC3, DDIT4, FGD4, FKBP5, MCL1, PER1, SRGN, TSC22D3 and TSPYL2, and wherein the three or more, e.g. three, four, five, six, seven, eight, nine, ten or eleven, target genes comprise one or more, e.g. one, two or three, target genes selected from AKAP13, FGD4 and TSPYL2. Thus the three or more target genes may comprise AKAP13 and two or more, e.g. two, three, four, five, six, seven, eight, nine, or ten, target genes selected from BCL6, BIRC3, DDIT4, FGD4, FKBP5, MCL1, PER1, SRGN, TSC22D3 and TSPYL2, or the three or more target genes may comprise FGD4 and two or more, e.g. two, three, four, five, six, seven, eight, nine, or ten, target genes selected from AKAP13, BCL6, BIRC3, DDIT4, FKBP5, MCL1, PER1, SRGN, TSC22D3 and TSPYL2, or the three or more target genes may comprise TSPYL2 and two or more, e.g. two, three, four, five, six, seven, eight, nine, or ten, target genes selected from AKAP13, BCL6, BIRC3, DDIT4, FGD4, FKBP5, MCL1, PER1, SRGN, and TSC22D3. Further advantages will be apparent to those of ordinary skill in the art upon reading and understanding the attached figures, the following description and, in particular, upon reading the detailed examples provided herein below.

It shall be understood that the method of claim 1, the apparatus of claim 9, the non-transitory storage medium of claim 10, the computer program of claim 11, the kits of claims 12 to 14, and the use of the kits of claim 15 have similar and/or identical preferred embodiments, in particular, as defined in the dependent claims.

It shall be understood that a preferred embodiment of the present invention can also be any combination of the dependent claims or above embodiments with the respective independent claim.

These and other aspects of the invention will be apparent from and elucidated with reference to the embodiments described hereinafter.

### Examples

The following examples merely illustrate particularly preferred methods and selected aspects in connection therewith. The teaching provided therein may be used for constructing several tests and/or kits, e.g., to detect, predict and/or diagnose the abnormal activity of the GR cellular signaling pathway. Furthermore, upon using methods as described herein drug prescription can advantageously be guided, drug response prediction and monitoring of drug efficacy (and/or adverse effects) can be made, drug resistance can be predicted and monitored, e.g., to select subsequent test(s) to be performed (like a companion diagnostic test). The following examples are not to be construed as limiting the scope of the present invention.

### 1. Mathematical Model Construction

As described in detail in the published international patent application WO 2013/011479 A2 (“Assessment of cellular signaling pathway activity using probabilistic modeling of target gene expression”), by constructing a probabilistic model, e.g., a Bayesian network model, and incorporating conditional probabilistic relationships between the expression levels of six or more target genes of a cellular signaling pathway, herein, the GR cellular signaling pathway, and the activity level of a transcription factor (TF) element, herein, the GR TF element, the TF element controlling transcription of the six or more target genes of the cellular signaling pathway, such a model may be used to determine the activity of the cellular signaling pathway with a high degree of accuracy. Moreover, the probabilistic model can be readily updated to incorporate additional knowledge obtained by later clinical studies, by adjusting the conditional probabilities and/or adding new nodes to the model to represent additional information sources. In this way, the probabilistic model can be updated as appropriate to embody the most recent medical knowledge.

In another easy to comprehend and interpret approach described in detail in the published international patent application WO 2014/102668 A2 (“Assessment of cellular signaling pathway activity using linear combination(s) of target gene expressions”), the activity of a cellular signaling pathway, herein, the GR cellular signaling pathway, may be determined by constructing and evaluating a linear or (pseudo-)linear model incorporating relationships between expression levels of six or more target genes of the cellular signaling pathway and the level of a transcription factor (TF) element, herein, the GR TF element, the TF element controlling transcription of the six or more target genes of the cellular signaling pathway, the model being based on one or more linear combination(s) of expression levels of the six or more target genes.

In both approaches, the expression levels of the six or more target genes may preferably be measurements of the level of mRNA, which can be the result of, e.g., (RT)-PCR and microarray techniques using probes associated with the target genes mRNA sequences, and of RNA-sequencing. In another embodiment, the expression levels of the six or more target genes can be measured by protein levels, e.g., the concentrations and/or activity of the protein(s) encoded by the target genes.

The aforementioned expression levels may optionally be converted in many ways that might or might not suit the application better. For example, four different transformations of the expression levels, e.g., microarray-based mRNA levels, may be:
“continuous data”, i.e., expression levels as obtained after preprocessing of microarrays using well known algorithms such as MAS5.0 and fRMA,
“z-score”, i.e., continuous expression levels scaled such that the average across all samples is 0 and the standard deviation is 1,
“discrete”, i.e., every expression above a certain threshold is set to 1 and below it to 0 (e.g., the threshold for a probeset may be chosen as the (weighted) median of its value in a set of a number of positive and the same number of negative clinical samples),
“fuzzy”, i.e., the continuous expression levels are converted to values between 0 and 1 using a sigmoid function of the following format: 1/(1+exp((thr−expr)/se)), with expr being the continuous expression levels, thr being the threshold as mentioned before and se being a softening parameter influencing the difference between 0 and 1.

One of the simplest linear models that can be constructed is a model having a node representing the transcription factor (TF) element, herein, the GR TF element, in a first layer and weighted nodes representing direct measurements of the target genes expression levels, e.g., by one probeset that is particularly highly correlated with the particular target gene, e.g., in microarray or (q)PCR experiments, in a second layer. The weights can be based either on calculations from a training data set or based on expert knowledge. This approach of using, in the case where possibly multiple expression levels are measured per target gene (e.g., in the case of microarray experiments, where one target gene can be measured with multiple probesets), only one expression level per target gene is particularly simple. A specific way of selecting the one expression level that is used for a particular target gene is to use the expression level from the probeset that is able to separate active and passive samples of a training data set the best. One method to determine this probeset is to perform a statistical test, e.g., the t-test, and select the probeset with the lowest p-value. The training data set's expression levels of the probeset with the lowest p-value is by definition the probeset with the least likely probability that the expression levels of the (known) active and passive samples overlap. Another selection method is based on odds-ratios. In such a model, one or more expression level(s) are provided for each of the six or more target genes and the one or more linear combination(s) comprise a linear combination including for each of the six or more target genes a weighted term, each weighted term being based on only one expression level of the one or more expression level(s) provided for the respective target gene. If only one expression level is chosen per target gene as described above, the model may be called a “most discriminant probe sets” model.

In an alternative to the “most discriminant probe sets” model, it is possible, in the case where possibly multiple expression levels are measured per target gene, to make use of all the expression levels that are provided per target gene. In such a model, one or more expression level(s) are provided for each of the six or more target genes and the one or more linear combination(s) comprise a linear combination of all expression levels of the one or more expression level(s) provided for the six or more target genes. In other words, for each of the six or more target genes, each of the one or more expression level(s) provided for the respective target gene may be weighted in the linear combination by its own (individual) weight. This variant may be called an “all probesets” model. It has an advantage of being relatively simple while making use of all the provided expression levels.

Both models as described above have in common that they are what may be regarded as “single-layer” models, in which the activity level of the TF element is calculated based on a linear combination of expression levels of the one or more probeset of the six or more target genes.

After the activity level of the TF element, herein, the GR TF element, has been determined by evaluating the respective model, the determined TF element activity level can be thresholded in order to infer the activity of the cellular signaling pathway, herein, the GR cellular signaling pathway. A preferred method to calculate such an appropriate threshold is by comparing the determined TF element activity levels wlc (weighted linear combination) of training samples known to have a passive cellular signaling pathway and training samples with an active cellular signaling pathway. A method that does so and also takes into account the variance in these groups is given by using a threshold

$$thr = \frac{{σ}_{{wlc}_{pas}} {μ}_{{wlc}_{act}} + {σ}_{{wlc}_{act}} {μ}_{{wlc}_{pas}}}{{σ}_{{wlc}_{pas}} + {σ}_{{wlc}_{act}}} \qquad ( 1 )$$

where σ and μ are the standard deviation and the mean of the determined TF element activity levels wlc for the training samples. In case only a small number of samples are available in the active and/or passive training samples, a pseudo count may be added to the calculated variances based on the average of the variances of the two groups:

$$\tilde{v} = \frac{{v}_{{wlc}_{act}} + {v}_{{wlc}_{pas}}}{2} \qquad ( 2 )$$

$${\tilde{v}}_{{wlc}_{act}} = \frac{x \tilde{v} + ( {n}_{act} - 1 ) {v}_{{wlc}_{act}}}{x + {n}_{act} - 1}$$

$${\tilde{v}}_{{wlc}_{pas}} = \frac{x \tilde{v} + ( {n}_{pas} - 1 ) {v}_{{wlc}_{pas}}}{x + {n}_{pas} - 1}$$

where ν is the variance of the determined TF element activity levels wlc of the groups, x is a positive pseudo count, e.g., 1 or 10, and n_{act} and nₚₒₛ are the number of active and passive samples, respectively. The standard deviation a can next be obtained by taking the square root of the variance v.

The threshold can be subtracted from the determined TF element activity levels wlc for ease of interpretation, resulting in a cellular signaling pathway's activity score in which negative values correspond to a passive cellular signaling pathway and positive values correspond to an active cellular signaling pathway.

As an alternative to the above-described “single-layer” models, a “two-layer” may also be used in an example. In such a model, a summary value is calculated for every target gene using a linear combination based on the measured intensities of its associated probesets (“first (bottom) layer”). The calculated summary value is subsequently combined with the summary values of the other target genes of the cellular signaling pathway using a further linear combination (“second (upper) layer”). Again, the weights can be either learned from a training data set or based on expert knowledge or a combination thereof. Phrased differently, in the “two-layer” model, one or more expression level(s) are provided for each of the six or more target genes and the one or more linear combination(s) comprise for each of the six or more target genes a first linear combination of all expression levels of the one or more expression level(s) provided for the respective target gene (“first (bottom) layer”). The model is further based on a further linear combination including for each of the six or more target genes a weighted term, each weighted term being based on the first linear combination for the respective target gene (“second (upper) layer”).

The calculation of the summary values can, in a preferred version of the “two-layer” model, include defining a threshold for each target gene using the training data and subtracting the threshold from the calculated linear combination, yielding the target gene summary. Here the threshold may be chosen such that a negative target gene summary value corresponds to a down-regulated target gene and that a positive target gene summary value corresponds to an up-regulated target gene. Also, it is possible that the target gene summary values are transformed using, e.g., one of the above-described transformations (fuzzy, discrete, etc.), before they are combined in the “second (upper) layer”.

After the activity level of the TF element has been determined by evaluating the “two-layer” model, the determined TF element activity level can be thresholded in order to infer the activity of the cellular signaling pathway, as described above.

In the following, the models described above are collectively denoted as “(pseudo-)linear” models. A more detailed description of the training and use of probabilistic models, e.g., a Bayesian network model, is provided in section 3 below.

### 2. Selection of Target Genes

A transcription factor (TF) is a protein complex (i.e., a combination of proteins bound together in a specific structure) or a protein that is able to regulate transcription from target genes by binding to specific DNA sequences, thereby controlling the transcription of genetic information from DNA to mRNA. The mRNA directly produced due to this action of the TF complex is herein referred to as a “direct target gene” (of the transcription factor). Cellular signaling pathway activation may also result in more secondary gene transcription, referred to as “indirect target genes”. In the following, (pseudo-)linear models or Bayesian network models (as exemplary mathematical models) comprising or consisting of direct target genes as direct links between cellular signaling pathway activity and mRNA level, are preferred, however the distinction between direct and indirect target genes is not always evident. Herein, a method to select direct target genes using a scoring function based on available scientific literature data is presented. Nonetheless, an accidental selection of indirect target genes cannot be ruled out due to limited information as well as biological variations and uncertainties.

Here we propose a list of GR target which are found to be transcribed upon binding of a dimeric protein complex consisting of GR to cellular DNA. The list was generated by manually curating scientific literature found using Pubmed (www.ncbi.nlm.nih.gov/pubmed/) and ScienceDirect (www.sciencedirect.com/). Collected evidence was classified into three categories: 1) GR binds to regulatory region; 2) Presence of a glucocorticoid response element (GRE) in the regulatory region; 3) Gene is differentially regulated by glucocorticoid.

An overall evidence score was computed by adding a normalized literature score (NLscore) and a normalized differential expression score (NDscore) as follows:
1. Normalized Literature score (NLscore): We computed literature scores (Lscores) for each literature evidence category using a weighted sum (see Tables 1 to 3). Per literature source, only the strongest evidence in each category (per gene) was used. The weight given to evidence produced in certain specific settings was corrected as indicated in Tables 1 to 3. We then computed the final Lscore by summing the Lscores obtained for each evidence category plus an extra point for genes with evidence in all three categories (complete evidence), or half a point for genes with evidence in two categories. The normalized literature score (NLscore) was then computed by dividing the Lscore of each gene by the maximum Lscore.
2. Normalized Differential expression score (NDscore): We estimated the differential gene expression score (Dscore) based on the magnitude and significance of the differential expression of the genes in question on a selection of Affymetrix HG1133Plus2 data sets. For each dataset, we calibrated a GR cellular signaling pathway model and used the calibration summary results to estimate differential expression magnitude. Only probesets that were significantly differentially expressed were taken into account. For each calibration set, we computed the proportion of significantly differentially expressed probesets, % sig, the average of odds ratios for the significantly differentially expressed probesets, av(OR), and the average differences in mean expression between GR cellular signaling pathway active and inactive calibration samples (of significantly differentially expressed probesets),

$$av ( diff ) = {av}_{ps} ( ( {\overline{on}}_{ps , set} ) - ( {\overline{off}}_{ps , set} ) ) , \qquad ( 3 )$$

where (o̅n̅_{ps,set}) is the average expression of active samples for a given probeset ps, on calibration set set and (o̅f̅f̅_{ps,set}) is the average expression of inactive samples for a given probeset ps, on calibration set set.

We then computed a differential expression score for each data set as:

$$Dscore = \% sig * av ( diff ) * {log}_{2} ( av ( OR ) ) * sign ( av ( diff ) ) , \qquad ( 4 )$$

and computed an overall score by adding the mean score obtained with the a blood dataset (GSE11336) and the score of the lung dataset (GSE17307). The normalized score was computed by dividing the absolute value of the Dscore of each gene by the maximum absolute value of the Dscores.

TABLE 1
Weights given depending on evidence strength for category
“GR binds to regulatory region” evidence.

| | Strength rank | Weight |
|---|---|---|
| Evidence type | | |
| ChIP/PCR | 1 | 1/1 |
| Luciferase assay, CAT assay, EMSA | | |
| assay, ChIP/CHIP, ChIP/reChIP, | | |
| ChIP/seq | 2 | 1/2 |
| H3K4me ChIP | 4 | 1/4 |
| Literature | 8 | 1/8 |
| Extra weight | | |
| GR binds near GR motif | | +half weight |
| Binding is weaker in mutated GR motif | | +half weight |

TABLE 2
Weights given depending on evidence strength for category
“GRE motif in the regulatory region” evidence.

| | Strength rank | Weight |
|---|---|---|
| Evidence type | | |
| Palindromic/perfect GRE (with sequence) | 1 | 1/1 |
| 1-2 mismatches/non specified GRE (with | 2 | 1/2 |
| sequence) | | |
| putative GRE/Half site GRE (with | 3 | 1/3 |
| sequence) | | |
| Perfect GRE (no sequence) | 3 | 1/3 |
| GRE (no sequence) | 4 | 1/4 |
| Half site GRE | 5 | 1/5 |
| putative GRE | 6 | 1/6 |
| Literature | 8 | 1/8 |
| Extra weight | | |
| GR binds near motif differentially | | +half weight |

TABLE 3
Weights given depending on evidence strength for category
“differential mRNA transcription” evidence.

| | Strength rank | Weight |
|---|---|---|
| Evidence type | | |
| PCR/Northern blot in CHX | 1 | 1 |
| PCR/Northern blot | 2 | 1/2 |
| Microarray in CHX | 2 | 1/2 |
| Microarray | 3 | 1/3 |
| RNAseq | 3 | 1/3 |
| Western blot/immune fluorescence | 4 | 1/4 |
| RNA PolII ChIP/PCR | 4 | 1/4 |
| RNA PolII ChIP/CHIP | 5 | 1/5 |
| literature | 8 | 1/8 |
| Extra weight | | |
| diff. expr. by nuclear GR | | +half weight |
| down regulated by anti-glucocorticoid | | +half weight |

Target genes were ranked according to level of evidence, and the top set of target genes was selected to construct the Bayesian model, see Table 4.

TABLE 4
GR target genes selected based on literature

FKBP5
TSC22D3
CAVIN2
PER1
PER2
PER3
NFKBIA
MCL1
FOXN2
BCL6
PSORS1C1
PSORS1C2
POU5F1
KLF4
TSPYL2
IGFBP1
BIRC3
ENTPD2
CIDEC
CEBPD
DUSP1
ZC3H12A
PARD6B
THBD
ELL2
EDN2
MT2A
ITPKC
OTULINL
ZFAND5
BAG3
SRGN
BAX
SGK1
TH
CHGA
SLC19A2
CXCL8
SCNN1A
DDIT4
CDKN1C
DNAJC15
NR3C1
AKAP13
FGD4

The Bayesian model was generated, as described

These preferred target gene lists were selected based on their capacity of separating a series of expected active samples from expected inactive samples from the calibration sets according to the following criteria:
(1) best AUC,
(2) best balanced accuracy,
(3) largest difference in activity between expected active and expected inactive samples, and
(4) smallest standard deviation of the average differences between inferred GR cellular signaling pathway activity of active and inactive samples (ground truth) from individual data sets. (The rationale behind this is that average difference in inferred GR cellular signaling pathway activity for active and inactive samples within a data set is preferably similar.).

Endoderm, ectoderm and mesoderm are developmental germ layers which in the embryo give rise to their own sets of cell types, while differentiation to other cell types becomes blocked by (epigenetic) chromatin changes. As a consequence, specific GR target genes that are not used in for example mesodermal derived cells, may not be accessible any more to an activated GR transcription factor in cell types from the mesodermal lineage. And vice versa for endodermal/ectodermal lineages. Since ectodermal and endodermal lineages are developmentally spoken closer together, differences in target gene expression are expected to be most prominent between mesodermal and ectodermal/endodermal derived cell types.

To enable development of a generic GR model and GR pathway activity test, it was necessary to investigate such differences in target gene expression between mesodermal and endodermal derived cell types, and subsequently identify a set of commonly expressed target genes.

The constructed Bayesian model was first calibrated on mesodermal-derived blood cells and on endodermal derived lung cells. Subsequently, to generate a generic model a set of common target genes was selected for construction of the generic model, and the model was calibrated on the combined set of data from samples of both endodermal and mesodermal origin.

### Generation of a Generic Model, to be Used on Both Endoderm Ectoderm-Derived Cell Types and Mesoderm-Derived Cell Types

For the generic model a subset of GR target genes was selected, that is, the GR target genes which contribute to the GR pathway activity score in both mesodermal and endo/ectodermal derived cell types, and that show consistent behavior in both cell types.

Initial selection of target genes resulted in the following Table 5:

TABLE 5
short list for calibration of model

BAG3
DNAJC15
NR3C1
PER2
SGK1
DDIT4
MCL1
AKAP13
BCL6
BIRC3
FGD4
FKBP5
PER1
SRGN
TSC22D3
TSPYL2

All genes have one or more probes with an absolute difference between activated and inactivated sample groups greater than 0.5 in both the blood and the lung model. The model was calibrated on samples of the blood and lung calibration dataset combined, where their numbers were balanced to avoid a bias towards one of the two cell types.

This set was evaluated on mesoderm samples (blood cells) and endoderm samples (lung cells). It was observed that not all target genes performed in a similar manner when comparing mesodermal and endodermal samples, therefore the list was further reduced to the following target genes which provide a uniform response on both germ layers (mesoderm and endoderm), see Table 6:

TABLE 6
validated genes for generic model.

AKAP13
BCL6
BIRC3
DDIT4
FGD4
FKBP5
MCL1
PER1
SRGN
TSC22D3
TSPYL2

### 3. Training and Using the Mathematical Model

Before the mathematical model can be used to infer the activity of the cellular signaling pathway, herein, the GR cellular signaling pathway, in a sample, the model must be appropriately trained.

If the mathematical pathway model is a probabilistic model, e.g., a Bayesian network model, based on conditional probabilities relating the activity level of the GR TF element and expression levels of six or more target genes of the GR cellular signaling pathway measured in the sample of the subject, the training may preferably be performed as described in detail in the published international patent application WO 2013/011479 A2 (“Assessment of cellular signaling pathway activity using probabilistic modeling of target gene expression”).

If the mathematical pathway model is based on one or more linear combination(s) of expression levels of six or more target genes of the GR cellular signaling pathway measured in the sample of the sample, the training may preferably be performed as described in detail in the published international patent application WO 2014/102668 A2 (“Assessment of cellular signaling pathway activity using linear combination(s) of target gene expressions”).

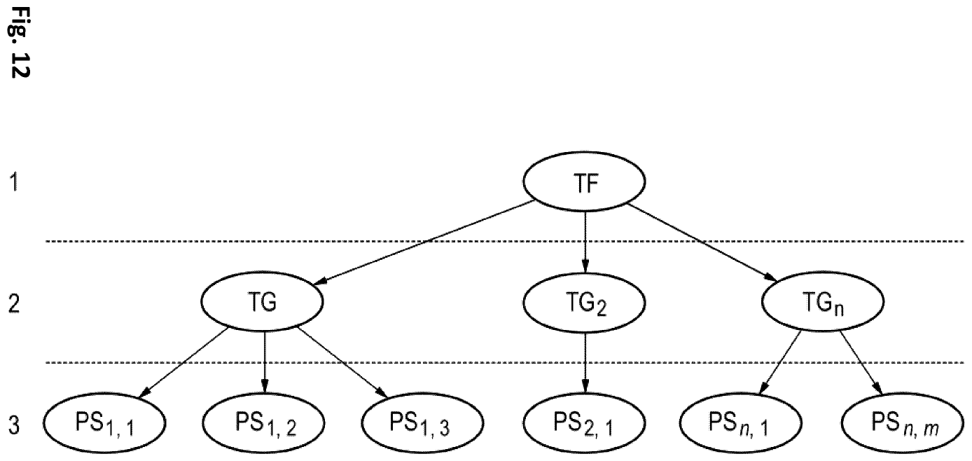

Herein, an exemplary Bayesian network model as shown in FIG. 12 was used to model the transcriptional program of the GR cellular signaling pathway in a simple manner. The model consists of three types of nodes: (a) a transcription factor (TF) element (with states “absent” and “present”) in a first layer 1; (b) target genes TG₁, TG₂, TGₙ (with states “down” and “up”) in a second layer 2, and; (c) measurement nodes linked to the expression levels of the target genes in a third layer 3. These can be microarray probesets PS_{1,1}, PS_{1,2}, PS_{1,3}, PS_{2,1}, PS_{n,1}, PS_{n,m} (with states “low” and “high”), as preferably used herein, but could also be other gene expression measurements such as RNAseq or RT-qPCR.

A suitable implementation of the mathematical model, herein, the exemplary Bayesian network model, is based on microarray data. The model describes (i) how the expression levels of the target genes depend on the activation of the TF element, and (ii) how probe set intensities, in turn, depend on the expression levels of the respective target genes. For the latter, probeset intensities may be taken from fRMA pre-processed Affymetrix HG-U133Plus2.0 microarrays, which are widely available from the Gene Expression Omnibus (GEO, www.ncbi.nlm.nih.gov/geo) and ArrayExpress (www.ebi.ac.uk/arrayexpress).

As the exemplary Bayesian network model is a simplification of the biology of a cellular signaling pathway, herein, the GR cellular signaling pathway, and as biological measurements are typically noisy, a probabilistic approach was opted for, i.e., the relationships between (i) the TF element and the target genes, and (ii) the target genes and their respective probesets, are described in probabilistic terms. Furthermore, it was assumed that the activity of the oncogenic cellular signaling pathway which drives tumor growth is not transiently and dynamically altered, but long term or even irreversibly altered. Therefore the exemplary Bayesian network model was developed for interpretation of a static cellular condition. For this reason complex dynamic cellular signaling pathway features were not incorporated into the model.

Once the exemplary Bayesian network model is built and calibrated (see below), the model can be used on microarray data of a new sample by entering the probeset measurements as observations in the third layer 3, and inferring backwards in the model what the probability must have been for the TF element to be “present”. Here, “present” is considered to be the phenomenon that the TF element is bound to the DNA and is controlling transcription of the cellular signaling pathway's target genes, and “absent” the case that the TF element is not controlling transcription. This probability is hence the primary read-out that may be used to indicate activity of the cellular signaling pathway, herein, the GR cellular signaling pathway, which can next be translated into the odds of the cellular signaling pathway being active by taking the ratio of the probability of it being active vs. it being passive (i.e., the odds are given by p/(1-p), where p is the predicted probability of the cellular signaling pathway being active).

In the exemplary Bayesian network model, the probabilistic relations have been made quantitative to allow for a quantitative probabilistic reasoning. In order to improve the generalization behavior across tissue types, the parameters describing the probabilistic relationships between (i) the TF element and the target genes have been carefully hand-picked. If the TF element is “absent”, it is most likely that the target gene is “down”, hence a probability of 0.95 is chosen for this, and a probability of 0.05 is chosen for the target gene being “up”. The latter (non-zero) probability is to account for the (rare) possibility that the target gene is regulated by other factors or that it is accidentally observed as being “up” (e.g. because of measurement noise). If the TF element is “present”, then with a probability of 0.70 the target gene is considered “up”, and with a probability of 0.30 the target gene is considered “down”. The latter values are chosen this way, because there can be several causes why a target gene is not highly expressed even though the TF element is present, e.g., because the gene's promoter region is methylated. In the case that a target gene is not up-regulated by the TF element, but down-regulated, the probabilities are chosen in a similar way, but reflecting the down-regulation upon presence of the TF element. The parameters describing the relationships between (ii) the target genes and their respective probesets have been calibrated on experimental data. For the latter, in this example, microarray data was used from patients samples which are known to have an active GR cellular signaling pathway whereas normal, healthy samples from a different data set were used as passive GR cellular signaling pathway samples, but this could also be performed using cell line experiments or other patient samples with known cellular signaling pathway activity status. The resulting conditional probability tables are given by:

A: for upregulated target genes

| | PS_{i,j} = low | PS_{i,j} = high |
|---|---|---|
| TGᵢ = down | $\frac{{AL}_{i , j} + 1}{A {L}_{i , j} + A {H}_{i , j} + 2}$ | $\frac{{AH}_{i , j} + 1}{A {L}_{i , j} + A {H}_{i , j} + 2}$ |
| TGᵢ = up | $\frac{P {L}_{i , j} + 1}{P {L}_{i , j} + P {H}_{i , j} + 2}$ | $\frac{P {H}_{i , j} + 1}{P {L}_{i , j} + P {H}_{i , j} + 2}$ |

B: for downregulated target genes

| | PS_{i,j} = low | PS_{i,j} = high |
|---|---|---|
| TGᵢ = down | $\frac{P {L}_{i , j} + 1}{P {L}_{i , j} + P {H}_{i , j} + 2}$ | $\frac{P {H}_{i , j} + 1}{P {L}_{i , j} + P {H}_{i , j} + 2}$ |
| TGᵢ = up | $\frac{{AL}_{i , j} + 1}{A {L}_{i , j} + A {H}_{i , j} + 2}$ | $\frac{{AH}_{i , j} + 1}{A {L}_{i , j} + A {H}_{i , j} + 2}$ |

In these tables, the variables AL_{i,j}, AH_{i,j}, PL_{i,j}, and PH_{i,j} indicate the number of calibration samples with an “absent” (A) or “present” (P) transcription complex that have a “low” (L) or “high” (H) probeset intensity, respectively. Dummy counts have been added to avoid extreme probabilities of 0 and 1.

To discretize the observed probeset intensities, for each probeset PS_{i,j} a threshold t_{i,j} was used, below which the observation is called “low”, and above which it is called “high”. This threshold has been chosen to be the (weighted) median intensity of the probeset in the used calibration data set. Due to the noisiness of microarray data, a fuzzy method was used when comparing an observed probeset intensity to its threshold, by assuming a normal distribution with a standard deviation of 0.25 (on a log 2 scale) around the reported intensity, and determining the probability mass below and above the threshold.

If instead of the exemplary Bayesian network described above, a (pseudo-)linear model as described in section 1 above was employed, the weights indicating the sign and magnitude of the correlation between the nodes and a threshold to call whether a node is either “absent” or “present” would need to be determined before the model could be used to infer cellular signaling pathway activity in a test sample. One could use expert knowledge to fill in the weights and the threshold a priori, but typically the model would be trained using a representative set of training samples, of which preferably the ground truth is known, e.g., expression data of probesets in samples with a known “present” transcription factor complex (=active cellular signaling pathway) or “absent” transcription factor complex (=passive cellular signaling pathway).

Known in the field are a multitude of training algorithms (e.g., regression) that take into account the model topology and changes the model parameters, here, the weights and the threshold, such that the model output, here, a weighted linear score, is optimized. Alternatively, it is also possible to calculate the weights directly from the observed expression levels without the need of an optimization algorithm.

A first method, named “black and white”-method herein, boils down to a ternary system, in which each weight is an element of the set {−1, 0, 1}. If this is put in a biological context, the −1 and 1 correspond to target genes or probesets that are down- and up-regulated in case of cellular signaling pathway activity, respectively. In case a probeset or target gene cannot be statistically proven to be either up- or down-regulated, it receives a weight of 0. In one example, a left-sided and right-sided, two sample t-test of the expression levels of the active cellular signaling pathway samples versus the expression levels of the samples with a passive cellular signaling pathway can be used to determine whether a probe or gene is up- or down-regulated given the used training data. In cases where the average of the active samples is statistically larger than the passive samples, i.e., the p-value is below a certain threshold, e.g., 0.3, the target gene or probeset is determined to be up-regulated. Conversely, in cases where the average of the active samples is statistically lower than the passive samples, the target gene or probeset is determined to be down-regulated upon activation of the cellular signaling pathway. In case the lowest p-value (left- or right-sided) exceeds the aforementioned threshold, the weight of the target gene or probeset can be defined to be 0.

A second method, named “log odds”-weights herein, is based on the logarithm (e.g., base e) of the odds ratio. The odds ratio for each target gene or probeset is calculated based on the number of positive and negative training samples for which the probeset/target gene level is above and below a corresponding threshold, e.g., the (weighted) median of all training samples. A pseudo-count can be added to circumvent divisions by zero. A further refinement is to count the samples above/below the threshold in a somewhat more probabilistic manner, by assuming that the probeset/target gene levels are e.g. normally distributed around its observed value with a certain specified standard deviation (e.g., 0.25 on a 2-log scale), and counting the probability mass above and below the threshold. Herein, an odds ratio calculated in combination with a pseudo-count and using probability masses instead of deterministic measurement values is called a “soft” odds ratio.

Further details regarding the inferring of cellular signaling pathway activity using mathematical modeling of target gene expression can be found in Verhaegh W. et al., “Selection of personalized patient therapy through the use of knowledge-based computational models that identify tumor-driving signal transduction pathways”, Cancer Research, Vol. 74, No. 11, 2014, pages 2936 to 2945.

### 4. Experimental Results

To demonstrate the GR models utility, here we present results obtained by applying various calibrated GR models to a series of Affymetrix HGU133Plus2.0 data sets. Activity read-outs are either presented as odds on:off (the odds of being active vs. being inactive), represented in a base 2 log scale, or as a normalized activity obtained by normalizing the log 2(odds on:off) to values between 0 and 100. The later normalized activity scale is useful when comparing activities of two different models that use a different number of target genes, target probesets, or calibration set, since the range of the log 2(odds on:off) of a model is highly dependent on those values.

### Generic Model Validation on Independent “Ground Truth” Datasets

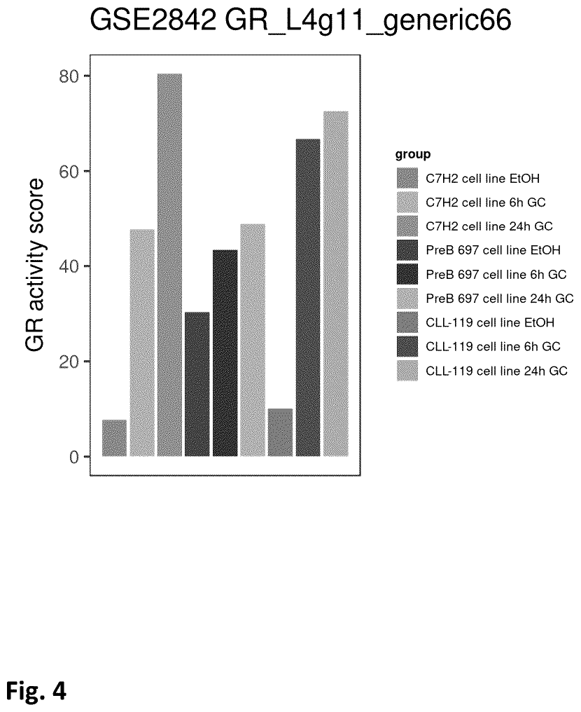

Following the calibration procedure for the generic model, the generic model was biologically validated on Affymetrix expression microarray (Affymetrix HG-U133 Plus2.0) a number of independent in vitro and in vivo experiments with a known GR pathway activity (FIG. 4-11). The cell types on which the generic model was validated represent derivatives of the endodermal (epithelial breast cancer cells) and mesodermal (blood cells and bone cells) embryonic germ layers. GR pathway activity scores are presented on a normalized 0-100 scale.

FIG. 4 shows validation on various leukemia blood cell lines. Addition of glucocorticoids (GC) for 6-24 hours resulted in an increase in GR pathway activity score. Ethanol (EthOH) was used as a negative control.

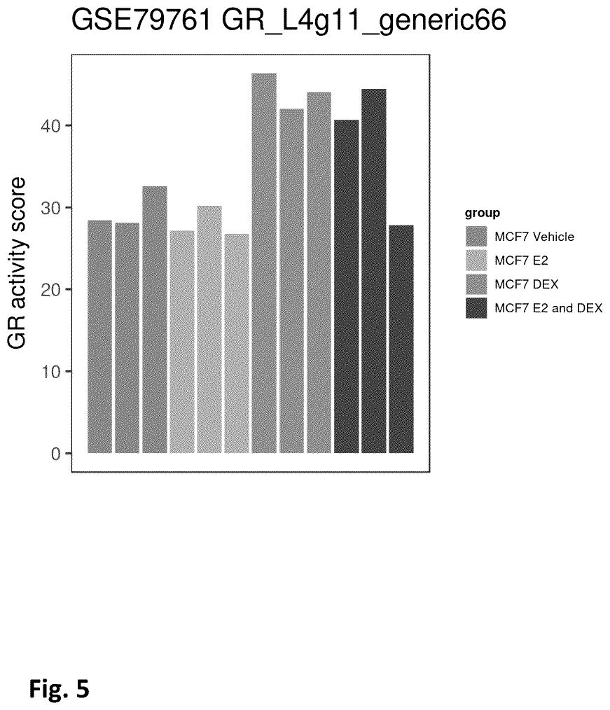

FIG. 5 shows validation on breast cancer epithelial cell line MCF7. Addition of glucocorticoids (dexamethasone (DEX)) resulted in an increase in GR pathway activity score. Estradiol (E2) is used as a control to demonstrate specificity of the pathway activity (estradiol is the ligand for the estrogen receptor pathway and should not activate GR).

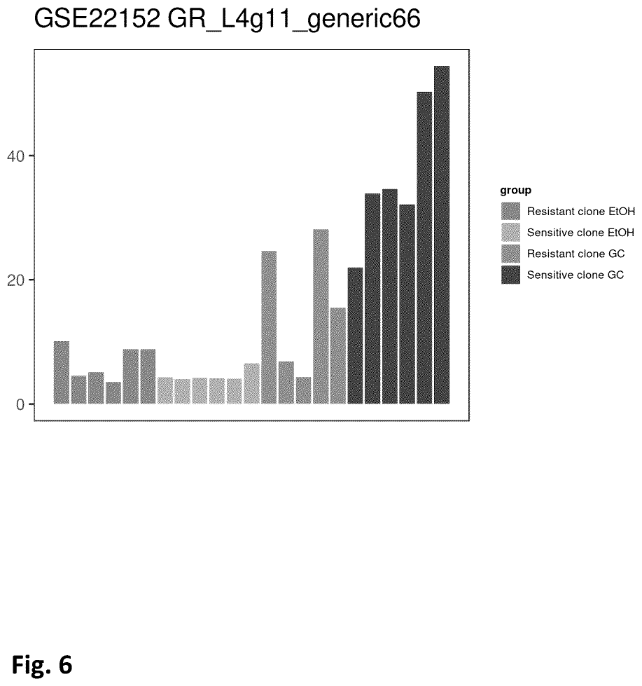

FIG. 6 shows validation on T-ALL blood cells. Addition of glucocorticoids (GC) to the glucocorticoid-resistant cell line resulted in a much lower increase in GR pathway activity score, than addition of GC to the sensitive cell line.

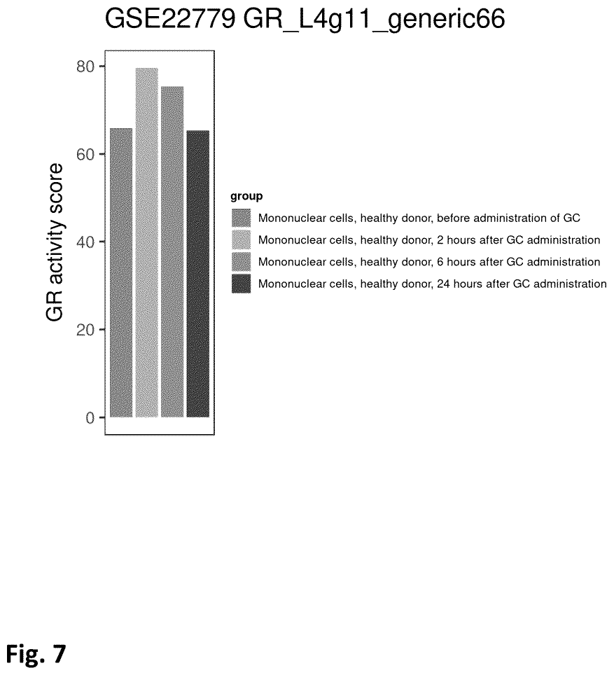

FIG. 7 shows validation on PBMCs from a healthy individual before and at several timepoints after administration of glucocorticoids. Addition of glucocorticoids (GC) in vivo resulted in a temporary increase in GR pathway activity score, which was maximal at 6 hours after administering glucocorticoids, and subsequently decreased to pre-treatment values at 24 hours after GC administration.

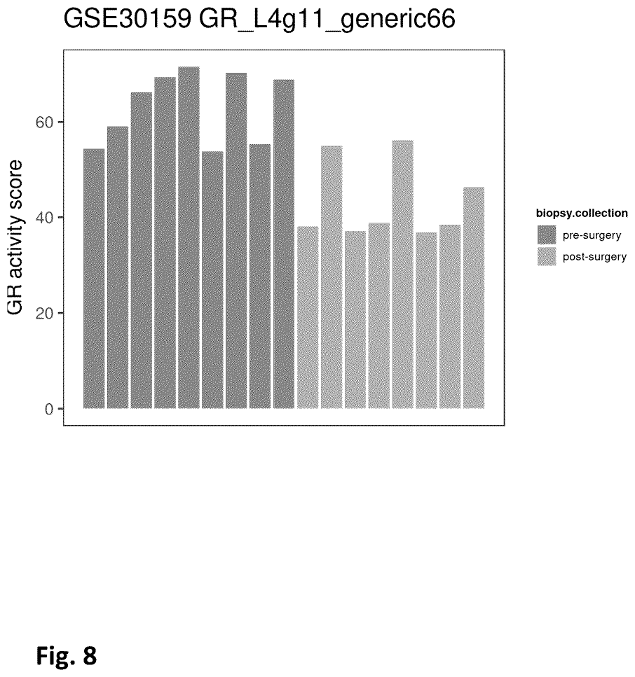

FIG. 8 shows validation on bone samples from patients with Cushing syndrome. Cushing syndrome is characterized by increased production of glucocorticoids by a tumor, which can be surgically removed to restore normal glucocorticoid levels. As can be seen, with the exception of patient 2, after surgery activity of the GR pathway decreases as expected.

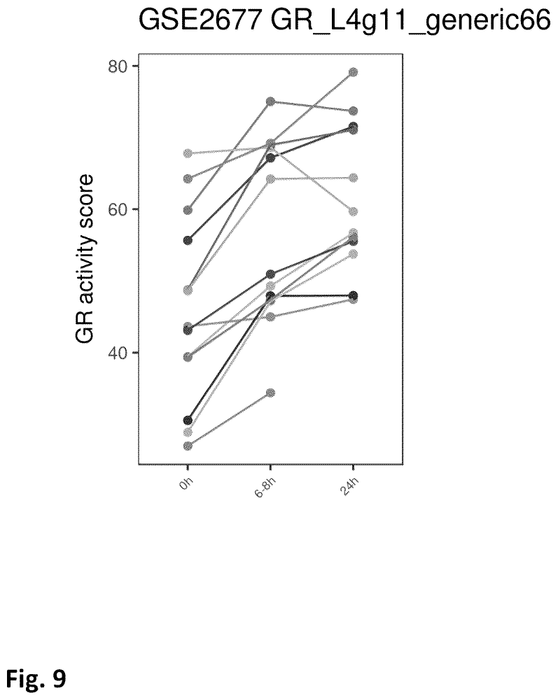

FIG. 9 shows Validation on blood derived lymphoblasts before and after in vivo treatment with GC. Glucocorticoids (GC) are standard treatment for lymphoid blood malignancies, e.g. pediatric acute lymphoblastic leukemia (ALL). Following administration of glucocorticoids, the GR pathway activity score increases in the lymphoblasts, with a stronger increase 24 hours after GC administration.

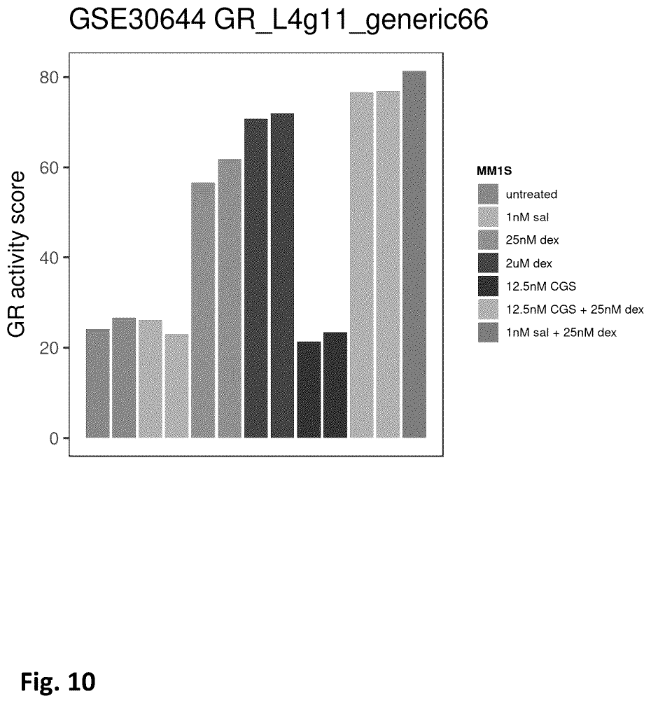

FIG. 10 shows validation on a multiple myeloma line (blood cells), treated with the GC dexamethasone. Dexamethasone induced an increase in GR pathway activity score, which was not affected by the combination with other drugs.

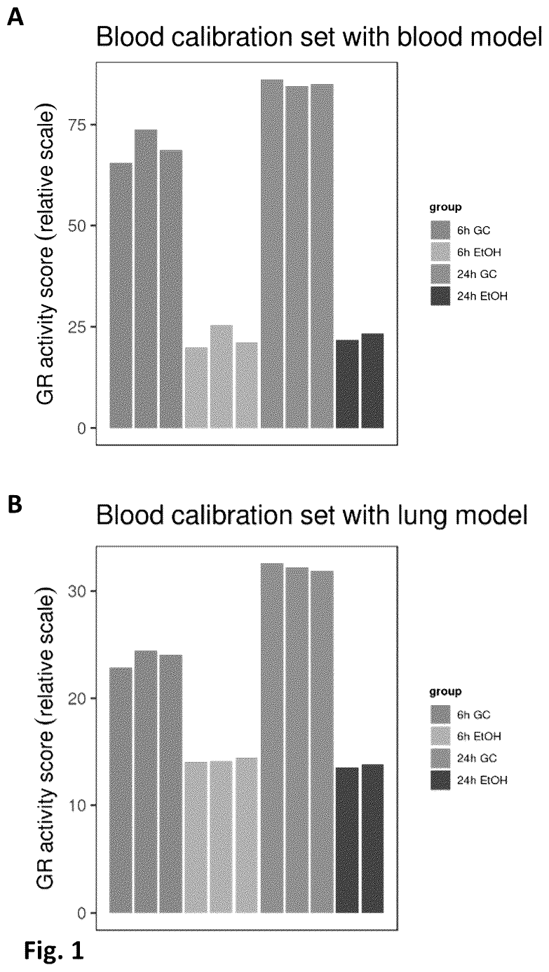

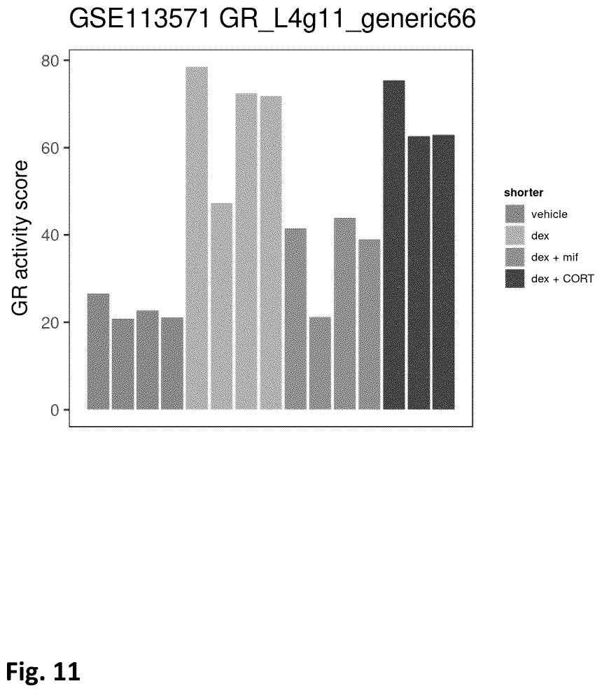

FIG. 11 shows validation results show that a GR pathway activity model calibrated on a cell type of endodermal origin can measure GR pathway activity on a cell type of endodermal origin (FIG. 1,2), performance is much lower on the cell types that originate from another germ layer than used for calibration. This problem was solved by developing a generic GR pathway model, based on the carefully selected set of GR target genes selected form the two separate models, that is, the target genes that performed best and consistent on cell types derived from both germ layers. This generic GR model performed very well on various cell and tissue types from different germ layer origin.

### 5. Further Information for Illustrating the Present Invention

### (1) Measuring Levels of Gene Expression

Data derived from the unique set of target genes described herein is further utilized to infer an activity of the GR cellular signaling pathway using the methods described herein.

Methods for analyzing gene expression levels in extracted samples are generally known. For example, methods such as Northern blotting, the use of PCR, nested PCR, quantitative real-time PCR (qPCR), RNA-seq, or microarrays can all be used to derive gene expression level data. All methods known in the art for analyzing gene expression of the target genes are contemplated herein.

Methods of determining the expression product of a gene using PCR based methods may be of particular use. In order to quantify the level of gene expression using PCR, the amount of each PCR product of interest is typically estimated using conventional quantitative real-time PCR (qPCR) to measure the accumulation of PCR products in real time after each cycle of amplification. This typically utilizes a detectible reporter such as an intercalating dye, minor groove binding dye, or fluorogenic probe whereby the application of light excites the reporter to fluoresce and the resulting fluorescence is typically detected using a CCD camera or photomultiplier detection system, such as that disclosed in U.S. Pat. No. 6,713,297 which is hereby incorporated by reference.

In some embodiments, the probes used in the detection of PCR products in the quantitative real-time PCR (qPCR) assay can include a fluorescent marker. Numerous fluorescent markers are commercially available. For example, Molecular Probes, Inc. (Eugene, Oreg.) sells a wide variety of fluorescent dyes. Non-limiting examples include Cy5, Cy3, TAMRA, R6G, R110, ROX, JOE, FAM, Texas Red™, and Oregon Green™ Additional fluorescent markers can include IDT ZEN Double-Quenched Probes with traditional 5′ hydrolysis probes in qPCR assays. These probes can contain, for example, a 5′ FAM dye with either a 3′ TAMRA Quencher, a 3′ Black Hole Quencher (BHQ, Biosearch Technologies), or an internal ZEN Quencher and 3′ Iowa Black Fluorescent Quencher (IBFQ).

Fluorescent dyes useful according to the invention can be attached to oligonucleotide primers using methods well known in the art. For example, one common way to add a fluorescent label to an oligonucleotide is to react an N-Hydroxysuccinimide (NHS) ester of the dye with a reactive amino group on the target. Nucleotides can be modified to carry a reactive amino group by, for example, inclusion of an allyl amine group on the nucleobase. Labeling via allyl amine is described, for example, in U.S. Pat. Nos. 5,476,928 and 5,958,691, which are incorporated herein by reference. Other means of fluorescently labeling nucleotides, oligonucleotides and polynucleotides are well known to those of skill in the art.

Other fluorogenic approaches include the use of generic detection systems such as SYBR-green dye, which fluoresces when intercalated with the amplified DNA from any gene expression product as disclosed in U.S. Pat. Nos. 5,436,134 and 5,658,751 which are hereby incorporated by reference.

Another useful method for determining target gene expression levels includes RNA-seq, a powerful analytical tool used for transcriptome analyses, including gene expression level difference between different physiological conditions, or changes that occur during development or over the course of disease progression.

Another approach to determine gene expression levels includes the use of microarrays for example RNA and DNA microarray, which are well known in the art. Microarrays can be used to quantify the expression of a large number of genes simultaneously.

### (2) Generalized Workflow for Determining the Activity of GR Cellular Signaling

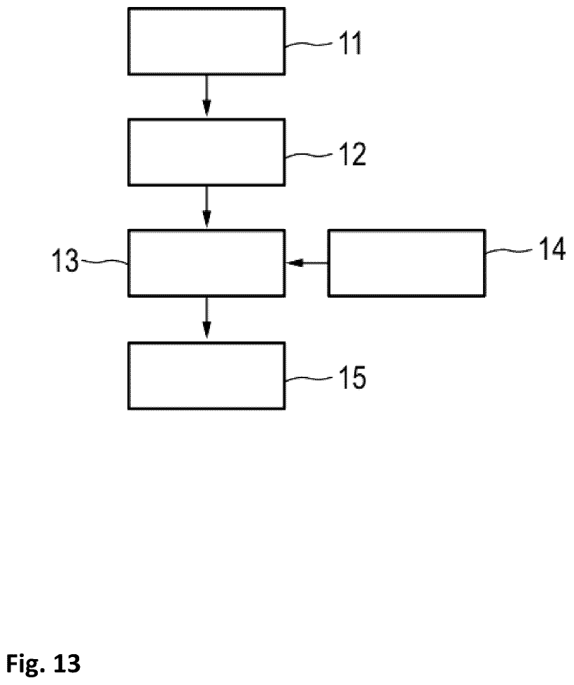

A flowchart exemplarily illustrating a process for inferring the activity of GR cellular signaling from a sample isolated from a subject is shown in FIG. 13. First, the mRNA from a sample is isolated (11). Second, the mRNA expression levels of a unique set of at least six or more GR target genes, as described herein, are measured (12) using methods for measuring gene expression that are known in the art. Next, an activity level of a GR transcription factor (TF) element (13) is determined using a calibrated mathematical pathway model (14) relating the expression levels of the six or more GR target genes to the activity level of the GR TF element. Finally, the activity of the GR cellular signaling pathway in the sample is inferred (15) based on the determined activity level of the GR TF element in the sample of the sample. For example, the GR cellular signaling pathway is determined to be active if the activity is above a certain threshold, and can be categorized as passive if the activity falls below a certain threshold.

### (3) Calibrated Mathematical Pathway Model

As contemplated herein, the expression levels of the unique set of six or more GR target genes described herein are used to determine an activity level of a GR TF element using a calibrated mathematical pathway model as further described herein. The calibrated mathematical pathway model relates the expression levels of the six or more GR target genes to the activity level of the GR TF element.

As contemplated herein, the calibrated mathematical pathway model is based on the application of a mathematical pathway model. For example, the calibrated mathematical pathway model can be based on a probabilistic model, for example, a Bayesian network model, or a linear or pseudo-linear model.

In an embodiment, the calibrated mathematical pathway model is a probabilistic model incorporating conditional probabilistic relationships relating the GR TF element and the expression levels of the six or more GR target genes. In an embodiment, the probabilistic model is a Bayesian network model.

In an alternative embodiment, the calibrated pathway mathematical model can be a linear or pseudo-linear model. In an embodiment, the linear or pseudo-linear model is a linear or pseudo-linear combination model as further described herein.

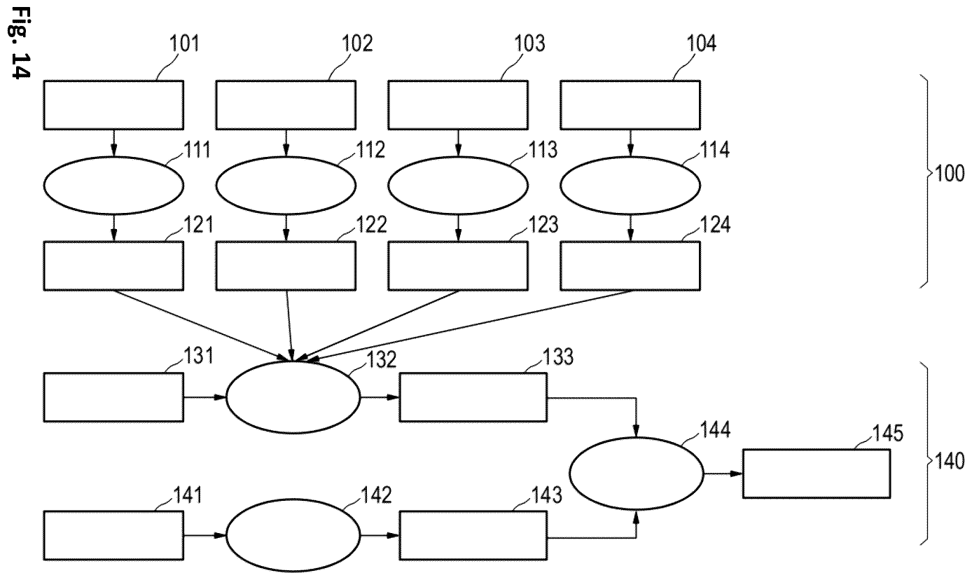

A flowchart exemplarily illustrating a process for generating a calibrated mathematical pathway model is shown in FIG. 14. As an initial step, the training data for the mRNA expression levels is collected and normalized. The data can be collected using, for example, microarray probeset intensities (101), real-time PCR Cq values (102), raw RNAseq reads (103), or alternative measurement modalities (104) known in the art. The raw expression level data can then be normalized for each method, respectively, by normalization using a normalization algorithm, for example, frozen robust multiarray analysis (fRMA) or MAS5.0 (111), normalization to average Cq of reference genes (112), normalization of reads into reads/fragments per kilobase of transcript per million mapped reads (RPKM/FPKM) (113), or normalization w.r.t. reference genes/proteins (114). This normalization procedure leads to a normalized probeset intensity (121), normalized Cq values (122), normalized RPKM/FPKM (123), or normalized measurement (124) for each method, respectively, which indicate target gene expression levels within the training samples.

Once the training data has been normalized, a training sample ID or IDs (131) is obtained and the training data of these specific samples is obtained from one of the methods for determining gene expression (132). The final gene expression results from the training sample are output as training data (133). All of the data from various training samples are incorporated to calibrate the model (including for example, thresholds, CPTs, for example in the case of the probabilistic or Bayesian network, weights, for example, in the case of the linear or pseudo-linear model, etc) (144). In addition, the pathway's target genes and measurement nodes (141) are used to generate the model structure for example, as described in FIG. 12 (142). The resulting model structure (143) of the pathway is then incorporated with the training data (133) to calibrate the model (144), wherein the gene expression levels of the target genes is indicative of the transcription factor element activity. As a result of the TF element determination in the training samples, a calibrated pathway model (145) is generated, which assigns the GR cellular signaling pathway activity for a subsequently examined sample of interest, for example from a subject with a cancer, based on the target gene expression levels in the training samples.

### (4) TF Element Determination

A flowchart exemplarily illustrating a process for determining an activity level of a TF element is shown in FIG. 15. The expression level data (test data) (163) from a sample extracted from a subject is input into the calibrated mathematical pathway model (145). The mathematical pathway model may be a probabilistic model, for example, a Bayesian network model, a linear model, or a pseudo-linear model.

The mathematical pathway model may be a probabilistic model, for example, a Bayesian network model, based on conditional probabilities relating the GR TF element and expression levels of the six or more target genes of the GR cellular signaling pathway measured in the sample of the subject, or the mathematical model may be based on one or more linear combination(s) of expression levels of the six or more target genes of the GR cellular signaling pathway measured in the sample of the subject. In particular, the determining of the activity of the GR cellular signaling pathway may be performed as disclosed in the published international patent application WO 2013/011479 A2 (“Assessment of cellular signaling pathway activity using probabilistic modeling of target gene expression”), the contents of which are herewith incorporated in their entirety. Briefly, the data is entered into a Bayesian network (BN) inference engine call (for example, a BNT toolbox) (154). This leads to a set of values for the calculated marginal BN probabilities of all the nodes in the BN (155). From these probabilities, the transcription factor (TF) node's probability (156) is determined and establishes the TF element's activity level (157).

Alternatively, the mathematical model may be a linear model. For example, a linear model can be used as described in the published international patent application WO 2014/102668 A2 (“Assessment of cellular signaling pathway activity using linear combination(s) of target gene expressions”), the contents of which are herewith incorporated in their entirety. Further details regarding the calculating/determining of cellular signaling pathway activity using mathematical modeling of target gene expression can also be found in Verhaegh W. et al., “Selection of personalized patient therapy through the use of knowledge-based computational models that identify tumor-driving signal transduction pathways”, Cancer Research, Vol. 74, No. 11, 2014, pages 2936-2945. Briefly, the data is entered into a calculated weighted linear combination score (w/c) (151). This leads to a set of values for the calculated weighted linear combination score (152). From these weighted linear combination scores, the transcription factor (TF) node's weighted linear combination score (153) is determined and establishes the TF's element activity level (157).

### (5) Procedure for Discretized Observables

A flowchart exemplarily illustrating a process for inferring activity of a GR cellular signaling pathway in a sample as a discretized observable is shown in FIG. 5. First, the test sample is extracted and given a test sample ID (161). Next, the test data for the mRNA expression levels is collected and normalized (162). The test data can be collected using the same methods as discussed for the training samples in FIG. 15, using microarray probeset intensities (101), real-time PCR Cq values (102), raw RNAseq reads (103), or an alternative measurement modalities (104). The raw expression level data can then be normalized for each method, respectively, by normalization using an algorithm, for example fRMA or MAS5.0 (111), normalization to average Cq of reference genes (112), normalization of reads into RPKM/FPKM (113), and normalization w.r.t. reference genes/proteins (114). This normalization procedure leads to a normalized probeset intensity (121), normalized Cq values (122), normalized RPKM/FPKM (123), or normalized measurement (124) for each method, respectively.

Once the test data has been normalized, the resulting test data (163) is analyzed in a thresholding step (164) based on the calibrated mathematical pathway model (145), resulting in the thresholded test data (165). In using discrete observables, in one non-limiting example, every expression above a certain threshold is, for example, given a value of 1 and values below the threshold are given a value of 0, or in an alternative embodiment, the probability mass above the threshold as described herein is used as a thresholded value. Based on the calibrated mathematical pathway model, this value represents the TF element's activity level (157), which is then used to calculate the cellular signaling pathway's activity (171). The final output gives the cellular signaling pathway's activity (172) in the sample.

### (6) Procedure for Continuous Observables

A flowchart exemplarily illustrating a process for inferring activity of a GR cellular signaling pathway in a sample as a continuous observable is shown in FIG. 6. First, the test sample is extracted and given a test sample ID (161). Next, the test data for the mRNA expression levels is collected and normalized (162). The test data can be collected using the same methods as discussed for the training samples in FIG. 5, using microarray probeset intensities (101), real-time PCR Cq values (102), raw RNAseq reads (103), or an alternative measurement modalities (104). The raw expression level data can then be normalized for each method, respectively, by normalization using an algorithm, for example fRMA (111), normalization to average Cq of reference genes (112), normalization of reads into RPKM/FPKM (113), and normalization w.r.t. reference genes/proteins (114). This normalization procedure leads to a normalized probeset intensity (121), normalized Cq values (122), normalized RPKM/FPKM (123), or normalized measurement (124) for each method, respectively.

Once the test data has been normalized, the resulting test data (163) is analyzed in the calibrated mathematical pathway model (145). In using continuous observables, as one non-limiting example, the expression levels are converted to values between 0 and 1 using a sigmoid function as described in further detail herein. The TF element determination as described herein is used to interpret the test data in combination with the calibrated mathematical pathway model, the resulting value represents the TF element's activity level (157), which is then used to calculate the cellular signaling pathway's activity (171). The final output gives the cellular signaling pathway's activity (172) in the sample.

### (7) Target Gene Expression Level Determination Procedure

A flowchart exemplary illustrating a process for deriving target gene expression levels from a sample extracted from a subject is shown in FIG. 7. In an exemplary embodiment, samples are received and registered in a laboratory. Samples can include, for example, Formalin-Fixed, Paraffin-Embedded (FFPE) samples (181) or fresh frozen (FF) samples (180). FF samples can be directly lysed (183). For FFPE samples, the paraffin can be removed with a heated incubation step upon addition of Proteinase K (182). Cells are then lysed (183), which destroys the cell and nuclear membranes which makes the nucleic acid (NA) available for further processing. The nucleic acid is bound to a solid phase (184) which could for example, be beads or a filter. The nucleic acid is then washed with washing buffers to remove all the cell debris which is present after lysis (185). The clean nucleic acid is then detached from the solid phase with an elution buffer (186). The DNA is removed by DNAse treatment to ensure that only RNA is present in the sample (187). The nucleic acid sample can then be directly used in the RT-qPCR sample mix (188). The RT-qPCR sample mixes contains the RNA sample, the RT enzyme to prepare cDNA from the RNA sample and a PCR enzyme to amplify the cDNA, a buffer solution to ensure functioning of the enzymes and can potentially contain molecular grade water to set a fixed volume of concentration. The sample mix can then be added to a multiwell plate (i.e., 96 well or 384 well plate) which contains dried RT-qPCR assays (189). The RT-qPCR can then be run in a PCR machine according to a specified protocol (190). An example PCR protocol includes i) 30 minutes at 50° C.; ii) 5 minutes at 95° C.; iii) 15 seconds at 95° C.; iv) 45 seconds at 60° C.; v) 50 cycles repeating steps iii and iv. The Cq values are then determined with the raw data by using the second derivative method (191). The Cq values are exported for analysis (192).

### (8) GR Mediated Diseases and Disorders and Methods of Treatment

As contemplated herein, the methods and apparatuses of the present invention can be utilized to assess GR cellular signaling pathway activity in a sample, for example, a sample obtained from a subject suspected of having, or having, a disease or disorder wherein the status of the GR signaling pathway is probative, either wholly or partially, of disease presence or progression. In an embodiment, provided herein is a method of treating a subject comprising receiving information regarding the activity status of a GR cellular signaling pathway derived from a sample extracted from the subject using the methods described herein and administering to the subject a GR inhibitor if the information regarding the activity of the GR cellular signaling pathway is indicative of an active GR signaling pathway. In a particular embodiment, the GR cellular signaling pathway activity indication is set at a cutoff value of odds of the GR cellular signaling pathway being active of 10:1, 5:1, 4:1, 2:1, 1:1, 1:2, 1:4, 1:5, 1:10.

This application describes several preferred embodiments. Modifications and alterations may occur to others upon reading and understanding the preceding detailed description. It is intended that the application is construed as including all such modifications and alterations insofar as they come within the scope of the appended claims or the equivalents thereof.

Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality.

A single unit or device may fulfill the functions of several items recited in the claims. The mere fact that certain measures are recited in mutually different dependent claims does not indicate that a combination of these measures cannot be used to advantage.

Calculations like the determination of the risk score performed by one or several units or devices can be performed by any other number of units or devices.

A computer program may be stored/distributed on a suitable medium, such as an optical storage medium or a solid-state medium, supplied together with or as part of other hardware, but may also be distributed in other forms, such as via the Internet or other wired or wireless telecommunication systems.

1-16. (canceled)

17. A method of treating, preventing, or ameliorating a disease in a subject in need thereof, the method comprising:
inferring activity of the GR cellular pathway in a sample obtained from a subject;
determining that the GR cellular signaling pathway activity in the sample is aberrant; and
administering, in response to determining that the GR cellular signaling pathway activity in the sample is aberrant, a GR cellular signaling pathway agonist or antagonist to the subject.

18. The method according to claim 17, wherein the disease is selected from cancer, a psychiatric disorder, an eye disorder, cardiovascular disease, inflammatory and immune related disorders, a respiratory disorder, a musculoskeletal disorders, a cutaneous inflammatory condition, or a metabolic disorder.

19. The method according to claim 18, wherein the metabolic disorder is Cushing's disease, Addison's disease, obesity, or hyperglycemia.

20. The method according to claim 17, wherein the agonist of the GR cellular signaling pathway is a natural or a synthetic glucocorticoid.

21. The method according to claim 20, wherein the natural glucocorticoid is selected from 11-Dehydrocorticosterone (11-oxocorticosterone, 17-deoxycortisone)=21-hydroxypregn-4-ene-3,11,20-trione, 11-Deoxycorticosterone (deoxycortone, desoxycortone; 21-hydroxyprogesterone)=21-hydroxypregn-4-ene-3,20-dione, 11-Deoxycortisol (cortodoxone, cortexolone)=17α,21-dihydroxypregn-4-ene-3,20-dione, 11-Ketoprogesterone (11-oxoprogesterone; Ketogestin)=pregn-4-ene-3,11,20-trione, 11β-Hydroxypregnenolone=3β,11β-dihydroxypregn-5-en-20-one, 11β-Hydroxyprogesterone (21-deoxycorticosterone)=11β-hydroxypregn-4-ene-3,20-dione, 11β,17α,21-Trihydroxypregnenolone=3β,11β,17α,21-tetrahydroxypregn-5-en-20-one, 17α,21-Dihydroxypregnenolone=3β,17α,21-trihydroxypregn-5-en-20-one, 17α-Hydroxypregnenolone=3β,17α-dihydroxypregn-5-en-20-one, 17α-Hydroxyprogesterone=17α-hydroxypregn-4-ene-3,11,20-trione, 18-Hydroxy-11-deoxycorticosterone=18,21-dihydroxypregn-4-ene-3,20-dione, 18-Hydroxycorticosterone=11β,18,21-trihydroxypregn-4-ene-3,20-dione, 18-Hydroxyprogesterone=18-hydroxypregn-4-ene-3,20-dione, 21-Deoxycortisol=11β,17α-dihydroxypregn-4-ene-3,20-dione, 21-Deoxycortisone=17α-hydroxypregn-4-ene-3,11,20-trione, 21-Hydroxypregnenolone (prebediolone)=3β,21-dihydroxypregn-5-en-20-one, Aldosterone=11β,21-dihydroxypregn-4-ene-3,18,20-trione, Corticosterone (17-deoxycortisol)=11β,21-dihydroxypregn-4-ene-3,20-dione, Cortisol (hydrocortisone)=11β,17α,21-trihydroxypregn-4-ene-3,20-dione, Cortisone=17α,21-dihydroxypregn-4-ene-3,11,20-trione, Pregnenolone=pregn-5-en-3β-ol-20-one, and Progesterone=pregn-4-ene-3,20-dione.

22. The method according to claim 21, wherein the synthetic glucocorticoid is selected from Flugestone (flurogestone)=9α-fluoro-11β,17α-dihydroxypregn-4-ene-3,20-dione, Fluorometholone=6α-methyl-9α-fluoro-11β,17α-dihydroxypregna-1,4-diene-3,20-dione, Medrysone (hydroxymethylprogesterone)=6α-methyl-11β-hydroxypregn-4-ene-3,20-dione, rebediolone acetate (21-acetoxypregnenolone)=3β,21-dihydroxypregn-5-en-20-one 21-acetate, Chloroprednisone=6α-chloro-17α,21-dihydroxypregna-1,4-diene-3,11,20-trione, Cloprednol=6-chloro-11β,17α,21-trihydroxypregna-1,4,6-triene-3,20-dione, Difluprednate=6α,9α-difluoro-11β,17α,21-trihydroxypregna-1,4-diene-3,20-dione 17α-butyrate 21-acetate, Fludrocortisone=9α-fluoro-11β,17α,21-trihydroxypregn-4-ene-3,20-dione, Fluocinolone=6α,9α-difluoro-11β,16α,17α,21-tetrahydroxypregna-1,4-diene-3,20-dione, Fluperolone=9α-fluoro-11β,17α,21-trihydroxy-21-methylpregna-1,4-diene-3,20-dione, Fluprednisolone=6α-fluoro-11β,17α,21-trihydroxypregna-1,4-diene-3,20-dione, Loteprednol=11β,17α,dihydroxy-21-oxa-21-chloromethylpregna-1,4-diene-3,20-dione, Methylprednisolone=6α-methyl-11β,17α,21-trihydroxypregna-1,4-diene-3,20-dione, Prednicarbate=11β,17α,21-trihydroxypregna-1,4-diene-3,20-dione 17α-ethylcarbonate 21-propionate, Prednisolone=11β,17α,21-trihydroxypregna-1,4-diene-3,20-dione, Prednisone=17α,21-dihydroxypregna-1,4-diene-3,11,20-trione, Tixocortol=11β,17α-dihydroxy-21-sulfanylpregn-4-ene-3,20-dione, Triamcinolone=9α-fluoro-11β,16α,17α,21-tetrahydroxypregna-1,4-diene-3,20-dione, Alclometasone=7α-chloro-11β,17α,21-trihydroxy-16α-methylpregna-1,4-diene-3,20-dione, Beclometasone=9α-chloro-11β,17α,21-trihydroxy-16β-methylpregna-1,4-diene-3,20-dione, Betamethasone=9α-fluoro-11β,17α,21-trihydroxy-16β-methylpregna-1,4-diene-3,20-dione, Clobetasol=9α-fluoro-11β,17α-dihydroxy-16β-methyl-21-chloropregna-1,4-diene-3,20-dione, Clobetasone=9α-fluoro-16β-methyl-17α-hydroxy-21-chloropregna-1,4-diene-3,11,20-trione, Clocortolone=6α-fluoro-9α-chloro-11β,21-dihydroxy-16α-methylpregna-1,4-diene-3,20-dione, Desoximetasone=9α-fluoro-11β,21-dihydroxy-16α-methylpregna-1,4-diene-3,20-dione, Dexamethasone=9α-fluoro-11β,17α,21-trihydroxy-16α-methylpregna-1,4-diene-3,20-dione, Diflorasone=6α,9α-difluoro-11β,17α,21-trihydroxy-16β-methylpregna-1,4-diene-3,20-dione, Difluocortolone=6α,9α-difluoro-11β,21-dihydroxy-16α-methylpregna-1,4-diene-3,20-dione, Fluclorolone=6α-fluoro-9α,11β-dichloro-16α,17α,21-trihydroxypregna-1,4-dien-3,20-dione, Flumetasone=6α,9α-difluoro-11β,17α,21-trihydroxy-16α-methylpregna-1,4-diene-3,20-dione, Fluocortin=6α-fluoro-11β,21-dihydroxy-16α-methylpregna-1,4-diene-3,20,21-trione, Fluocortolone=6α-fluoro-11β,21-dihydroxy-16α-methylpregna-1,4-diene-3,20-dione, Fluprednidene=9α-fluoro-11β,17α,21-trihydroxy-16-methylenepregna-1,4-diene-3,20-dione, Fluticasone=6α,9α-difluoro-11β,17α-dihydroxy-16α-methyl-21-thia-21-fluoromethylpregna-1,4-dien-3,20-dione, Fluticasone furoate=6α,9α-difluoro-11β,17α-dihydroxy-16α-methyl-21-thia-21-fluoromethylpregna-1,4-dien-3,20-dione 17α-(2-furoate), Halometasone=2-chloro-6α,9α-difluoro-11β,17α,21-trihydroxy-16α-methylpregna-1,4-diene-3,20-dione, Meprednisone=16β-methyl-17α,21-dihydroxypregna-1,4-diene-3,11,20-trione,Mometasone=9α,21-dichloro-11β,17α-dihydroxy-16α-methylpregna-1,4-diene-3,20-dione, Mometasone furoate=9α,21-dichloro-11β,17α-dihydroxy-16α-methylpregna-1,4-diene-3,20-dione 17α-(2-furoate), Paramethasone=6α-fluoro-11β,17α,21-trihydroxy-16α-methylpregna-1,4-diene-3,20-dione, Prednylidene=11β,17α,21-trihydroxy-16-methylenepregna-1,4-diene-3,20-dione, Rimexolone=11β-hydroxy-16α,17α,21-trimethylpregna-1,4-dien-3,20-dione, Ulobetasol (halobetasol)=6α,9α-difluoro-11β,17α-dihydroxy-16β-methyl-21-chloropregna-1,4-diene-3,20-dione, Amcinonide=9α-fluoro-11β,16α,17α,21-tetrahydroxypregna-1,4-diene-3,20-dione cyclic 16α,17α-acetal with cyclopentanone, 21-acetate, Budesonide=11β,16α,17α,21-tetrahydroxypregna-1,4-diene-3,20-dione cyclic 16α,17α-acetal with butyraldehyde, Ciclesonide=11β,16α,17α,21-tetrahydroxypregna-1,4-diene-3,20-dione cyclic 16α,17α-acetal with (R)-cyclohexanecarboxaldehyde, 21-isobutyrate, Deflazacort=11β,21-dihydroxy-2′-methyl-5′H-pregna-1,4-dieno[17,16-d]oxazole-3,20-dione 21-acetate, Desonide=11β,16α,17α,21-tetrahydroxypregna-1,4-diene-3,20-dione cyclic 16α,17α-acetal with acetone, Formocortal (fluoroformylone)=3-(2-chloroethoxy)-9α-fluoro-11β,16α,17α,21-tetrahydroxy-20-oxopregna-3,5-diene-6-carboxaldehyde cyclic 16α,17α-acetal with acetone, 21-acetate, Fluclorolone acetonide (flucloronide)=6α-fluoro-9α,11β-dichloro-16α,17α,21-trihydroxypregna-1,4-dien-3,20-dione cyclic 16α,17α-acetal with acetone, Fludroxycortide (flurandrenolone, flurandrenolide)=6α-fluoro-11β,16α,17α,21-tetrahydroxypregn-4-ene-3,20-dione cyclic 16α,17α-acetal with acetone, Flunisolide=6α-fluoro-11β,16α,17α,21-tetrahydroxypregna-1,4-diene-3,20-dione cyclic 16α,17α-acetal with acetone, Fluocinolone acetonide=6α,9α-difluoro-11β,16α,17α,21-tetrahydroxypregna-1,4-diene-3,20-dione cyclic 16α,17α-acetal with acetone, Fluocinonide=6α,9α-difluoro-11β,16α,17α,21-tetrahydroxypregna-1,4-diene-3,20-dione cyclic 16α,17α-acetal with acetone, 21-acetate, Halcinonide=9α-fluoro-11β,16α,17α-trihydroxy-21-chloropregn-4-ene-3,20-dione cyclic 16α,17α-acetal with acetone, Triamcinolone acetonide=9α-fluoro-11β,16α,17α,21-tetrahydroxypregna-1,4-diene-3,20-dione cyclic 16α,17α-acetal with acetone, Cortivazol=6,16α-dimethyl-11β,17α,21-trihydroxy-2′-phenyl[3,2-c]pyrazolopregna-4,6-dien-20-one 21-acetate, and RU-28362=6-methyl-11β,17β-dihydroxy-17α-(1-propynyl)androsta-1,4,6-trien-3-one.

23. The method according to claim 17, wherein the antagonist of the GR cellular signaling pathway is selected from RTI 3021-012, RTI 3021-022, Cyproterone acetate, RU-486, RU-43044, 11-Monoaryl steroids, 11,21 Bisaryl Steroids, 11β-Substituted Steroids, Bridged 11β-Aryl Steroids, 10β-Substituted Steroids, Org 34517, Org 36410, Org 34850, 11β-Aryl Conjugates of Mifepristone, SAR for 11β-Aryl Substituents in Mifepristone, 11β-Aryl Substituted Mifepristone Derivatives, A-348441, Steroidal Phosphonates, Phosphorus-Containing Mifepristone Analogs, Octahydrophenanthrenes, CP-409069, CP-394531, Sprirocyclic Dihydropyridines, CP-47255, Triphenylmethanes and Diaryl ethers, KB285, AL-438, Chromenes, Dibenzyl anilines, Azadecalins, Aryl Pyrazolo Azadecalins, 8a-Benzyl Isoquinolones, Fluorocortivazol, or a combination thereof.

24. The method according to claim 17, wherein the activity of the GR cellular signaling pathway is inferred by a computer-implemented method performed by a digital processing device, wherein the inferring comprises:
receiving expression levels of six or more target genes of the GR cellular signaling pathway measured in the sample;
determining an activity level of a GR transcription factor (TF) element in the sample, the GR TF element controlling transcription of the six or more GR target genes, the determining being based on evaluating a calibrated mathematical pathway model relating the expression levels of the six or more GR target genes to the activity level of the GR TF element; and
inferring the activity of the GR cellular signaling pathway in the sample based on the determined activity level of the GR TF element in the sample;
wherein the calibrated mathematical pathway model is not germline specific and the six or more target genes are selected from the group consisting of: AKAP13, BCL6, BIRC3, DDIT4, FGD4, FKBP5, MCL1, PER1, SRGN, TSC22D3 and TSPYL2.

25. The method of claim 24, further comprising:
recommending prescribing a drug for a subject that corrects for undesired activity level operation of the GR cellular signaling pathway, wherein the recommending is performed if the GR cellular signaling pathway is determined to be operating at an undesired activity level in the subject based on the inferred activity of the GR cellular signaling pathway.

26. The method of claim 24, wherein the aberrant activity of the GR cellular signaling pathway is an activity level in which the GR cellular signaling pathway operates as a promoter of, or fails to inhibit, or is an indication of one or more of the following disorders: cancer, a psychiatric disorder, an eye disorder, cardiovascular disease, inflammatory and immune related disorders, a respiratory disorder, a musculoskeletal disorders, a cutaneous inflammatory condition, or a metabolic disorder.

27. The method of claim 24, wherein the calibrated mathematical pathway model is a probabilistic model based on conditional probabilities relating the activity level of the GR TF element and the expression levels of the six or more GR target genes, or wherein the mathematical pathway model is based on one or more (pseudo-) linear combination(s) of the expression levels of the six or more GR target genes.

28. The method of claim 24, wherein the method further comprises determining the expression levels of the six or more GR target genes in the sample, and wherein said determining the expression levels of the six or more GR target genes in the sample is based on mRNA extracted from the sample.

29. A method of predicting a response to a drug in a subject having a disease, the method comprising:
determining the expression levels of six or more target genes of the GR cellular signaling pathway measured in a sample;
determining an activity level of a GR transcription factor (TF) element in the sample, the GR TF element controlling transcription of the six or more GR target genes, the determining being based on evaluating a calibrated mathematical pathway model relating the expression levels of the six or more GR target genes to the activity level of the GR TF element;
inferring the activity of the GR cellular signaling pathway in the sample based on the determined activity level of the GR TF element in the sample, wherein the inferred activity of the GR cellular signaling pathway activity in the sample is determined to be aberrant;
wherein the calibrated mathematical pathway model is not germline specific and the six or more target genes are selected from the group consisting of: AKAP13, BCL6, BIRC3, DDIT4, FGD4, FKBP5, MCL1, PER1, SRGN, TSC22D3 and TSPYL2; and
predicting the response to a drug based on the inferred activity of the GR cellular signaling pathway in the sample.

30. The method according to claim 29, wherein said determining the expression levels of the six or more GR target genes in the sample is based on mRNA extracted from the sample.

31. The method according to claim 30, wherein the expression levels are analyzed using a method selected from Northern blotting, PCR, nested PCR, quantitative real-time PCR (qPCR), RNA-seq, or microarrays.

32. The method according to claim 29, wherein the method comprises recommending administering a GR cellular signaling pathway agonist or antagonist to the subject when the GR cellular signaling pathway activity in the sample is aberrant.

33. The method according to claim 32, wherein the antagonist of the GR cellular signaling pathway is selected from RTI 3021-012, RTI 3021-022, Cyproterone acetate, RU-486, RU-43044, 11-Monoaryl steroids, 11,21 Bisaryl Steroids, 11β-Substituted Steroids, Bridged 11β-Aryl Steroids, 10β-Substituted Steroids, Org 34517, Org 36410, Org 34850, 110-Aryl Conjugates of Mifepristone, SAR for 11β-Aryl Substituents in Mifepristone, 11β-Aryl Substituted Mifepristone Derivatives, A-348441, Steroidal Phosphonates, Phosphorus-Containing Mifepristone Analogs, Octahydrophenanthrenes, CP-409069, CP-394531, Sprirocyclic Dihydropyridines, CP-47255, Triphenylmethanes and Diaryl ethers, KB285, AL-438, Chromenes, Dibenzyl anilines, Azadecalins, Aryl Pyrazolo Azadecalins, 8a-Benzyl Isoquinolones, Fluorocortivazol, or combinations thereof.

34. The method according to claim 32, wherein the agonist of the GR cellular signaling pathway is a natural or a synthetic glucocorticoid.

35. The method according to claim 34, wherein the natural glucocorticoids is selected from 11-Dehydrocorticosterone (11-oxocorticosterone, 17-deoxycortisone)=21-hydroxypregn-4-ene-3,11,20-trione, 11-Deoxycorticosterone (deoxycortone, desoxycortone; 21-hydroxyprogesterone)=21-hydroxypregn-4-ene-3,20-dione, 11-Deoxycortisol (cortodoxone, cortexolone)=17α,21-dihydroxypregn-4-ene-3,20-dione, 11-Ketoprogesterone (11-oxoprogesterone; Ketogestin)=pregn-4-ene-3,11,20-trione, 11β-Hydroxypregnenolone=3β,11β-dihydroxypregn-5-en-20-one, 11β-Hydroxyprogesterone (21-deoxycorticosterone)=11β-hydroxypregn-4-ene-3,20-dione, 11β,17α,21-Trihydroxypregnenolone=3β,11β,17α,21-tetrahydroxypregn-5-en-20-one, 17α,21-Dihydroxypregnenolone=3β,17α,21-trihydroxypregn-5-en-20-one, 17α-Hydroxypregnenolone=3β,17α-dihydroxypregn-5-en-20-one, 17α-Hydroxyprogesterone=17α-hydroxypregn-4-ene-3,11,20-trione, 18-Hydroxy-11-deoxycorticosterone=18,21-dihydroxypregn-4-ene-3,20-dione, 18-Hydroxycorticosterone=11β,18,21-trihydroxypregn-4-ene-3,20-dione, 18-Hydroxyprogesterone=18-hydroxypregn-4-ene-3,20-dione, 21-Deoxycortisol=11β,17α-dihydroxypregn-4-ene-3,20-dione, 21-Deoxycortisone=17α-hydroxypregn-4-ene-3,11,20-trione, 21-Hydroxypregnenolone (prebediolone)=30,21-dihydroxypregn-5-en-20-one, Aldosterone=11β,21-dihydroxypregn-4-ene-3,18,20-trione, Corticosterone (17-deoxycortisol)=11β,21-dihydroxypregn-4-ene-3,20-dione, Cortisol (hydrocortisone)=11β,17α,21-trihydroxypregn-4-ene-3,20-dione, Cortisone=17α,21-dihydroxypregn-4-ene-3,11,20-trione, Pregnenolone=pregn-5-en-3β-ol-20-one and Progesterone=pregn-4-ene-3,20-dione.

36. The method according to claim 34, wherein the synthetic glucocorticoid is selected from Flugestone (flurogestone)=9α-fluoro-11β,17α-dihydroxypregn-4-ene-3,20-dione, Fluorometholone=6α-methyl-9α-fluoro-11β,17α-dihydroxypregna-1,4-diene-3,20-dione, Medrysone (hydroxymethylprogesterone)=6α-methyl-11β-hydroxypregn-4-ene-3,20-dione, rebediolone acetate (21-acetoxypregnenolone)=3β,21-dihydroxypregn-5-en-20-one 21-acetate, Chloroprednisone=6α-chloro-17α,21-dihydroxypregna-1,4-diene-3,11,20-trione, Cloprednol=6-chloro-11β,17α,21-trihydroxypregna-1,4,6-triene-3,20-dione, Difluprednate=6α,9α-difluoro-11β,17α,21-trihydroxypregna-1,4-diene-3,20-dione 17α-butyrate 21-acetate, Fludrocortisone=9α-fluoro-11β,17α,21-trihydroxypregn-4-ene-3,20-dione, Fluocinolone=6α,9α-difluoro-11β,16α,17α,21-tetrahydroxypregna-1,4-diene-3,20-dione, Fluperolone=9α-fluoro-11β,17α,21-trihydroxy-21-methylpregna-1,4-diene-3,20-dione, Fluprednisolone=6α-fluoro-11β,17α,21-trihydroxypregna-1,4-diene-3,20-dione, Loteprednol=11β,17α,dihydroxy-21-oxa-21-chloromethylpregna-1,4-diene-3,20-dione, Methylprednisolone=6α-methyl-11β,17α,21-trihydroxypregna-1,4-diene-3,20-dione, Prednicarbate=11β,17α,21-trihydroxypregna-1,4-diene-3,20-dione 17α-ethylcarbonate 21-propionate, Prednisolone=11β,17α,21-trihydroxypregna-1,4-diene-3,20-dione, Prednisone=17α,21-dihydroxypregna-1,4-diene-3,11,20-trione, Tixocortol=11β,17α-dihydroxy-21-sulfanylpregn-4-ene-3,20-dione, Triamcinolone=9α-fluoro-11β,16α,17α,21-tetrahydroxypregna-1,4-diene-3,20-dione, Alclometasone=7α-chloro-11β,17α,21-trihydroxy-16α-methylpregna-1,4-diene-3,20-dione, Beclometasone=9α-chloro-11β,17α,21-trihydroxy-16β-methylpregna-1,4-diene-3,20-dione, Betamethasone=9α-fluoro-11β,17α,21-trihydroxy-16β-methylpregna-1,4-diene-3,20-dione, Clobetasol=9α-fluoro-11β,17α-dihydroxy-16β-methyl-21-chloropregna-1,4-diene-3,20-dione, Clobetasone=9α-fluoro-16β-methyl-17α-hydroxy-21-chloropregna-1,4-diene-3,11,20-trione, Clocortolone=6α-fluoro-9α-chloro-11β,21-dihydroxy-16α-methylpregna-1,4-diene-3,20-dione, Desoximetasone=9α-fluoro-11β,21-dihydroxy-16α-methylpregna-1,4-diene-3,20-dione, Dexamethasone=9α-fluoro-11β,17α,21-trihydroxy-16α-methylpregna-1,4-diene-3,20-dione, Diflorasone=6α,9α-difluoro-11β,17α,21-trihydroxy-16β-methylpregna-1,4-diene-3,20-dione, Difluocortolone=6α,9α-difluoro-11β,21-dihydroxy-16α-methylpregna-1,4-diene-3,20-dione, Fluclorolone=6α-fluoro-9α,11β-dichloro-16α,17α,21-trihydroxypregna-1,4-dien-3,20-dione, Flumetasone=6α,9α-difluoro-11β,17α,21-trihydroxy-16α-methylpregna-1,4-diene-3,20-dione, Fluocortin=6α-fluoro-11β,21-dihydroxy-16α-methylpregna-1,4-diene-3,20,21-trione, Fluocortolone=6α-fluoro-11β,21-dihydroxy-16α-methylpregna-1,4-diene-3,20-dione, Fluprednidene=9α-fluoro-11β,17α,21-trihydroxy-16-methylenepregna-1,4-diene-3,20-dione, Fluticasone=6α,9α-difluoro-11β,17α-dihydroxy-16α-methyl-21-thia-21-fluoromethylpregna-1,4-dien-3,20-dione, Fluticasone furoate=6α,9α-difluoro-11β,17α-dihydroxy-16α-methyl-21-thia-21-fluoromethylpregna-1,4-dien-3,20-dione 17α-(2-furoate), Halometasone=2-chloro-6α,9α-difluoro-11β,17α,21-trihydroxy-16α-methylpregna-1,4-diene-3,20-dione, Meprednisone=16β-methyl-17α,21-dihydroxypregna-1,4-diene-3,11,20-trione,Mometasone=9α,21-dichloro-11β,17α-dihydroxy-16α-methylpregna-1,4-diene-3,20-dione, Mometasone furoate=9α,21-dichloro-11β,17α-dihydroxy-16α-methylpregna-1,4-diene-3,20-dione 17α-(2-furoate), Paramethasone=6α-fluoro-11β,17α,21-trihydroxy-16α-methylpregna-1,4-diene-3,20-dione, Prednylidene=11β,17α,21-trihydroxy-16-methylenepregna-1,4-diene-3,20-dione, Rimexolone=11β-hydroxy-16α,17α,21-trimethylpregna-1,4-dien-3,20-dione, Ulobetasol (halobetasol)=6α,9α-difluoro-11β,17α-dihydroxy-16β-methyl-21-chloropregna-1,4-diene-3,20-dione, Amcinonide=9α-fluoro-11β,16α,17α,21-tetrahydroxypregna-1,4-diene-3,20-dione cyclic 16α,17α-acetal with cyclopentanone, 21-acetate, Budesonide=11β,16α,17α,21-tetrahydroxypregna-1,4-diene-3,20-dione cyclic 16α,17α-acetal with butyraldehyde, Ciclesonide=11β,16α,17α,21-tetrahydroxypregna-1,4-diene-3,20-dione cyclic 16α,17α-acetal with (R)-cyclohexanecarboxaldehyde, 21-isobutyrate, Deflazacort=11β,21-dihydroxy-2′-methyl-5′H-pregna-1,4-dieno[17,16-d]oxazole-3,20-dione 21-acetate, Desonide=11β,16α,17α,21-tetrahydroxypregna-1,4-diene-3,20-dione cyclic 16α,17α-acetal with acetone, Formocortal (fluoroformylone)=3-(2-chloroethoxy)-9α-fluoro-11β,16α,17α,21-tetrahydroxy-20-oxopregna-3,5-diene-6-carboxaldehyde cyclic 16α,17α-acetal with acetone, 21-acetate, Fluclorolone acetonide (flucloronide)=6α-fluoro-9α,11β-dichloro-16α,17α,21-trihydroxypregna-1,4-dien-3,20-dione cyclic 16α,17α-acetal with acetone, Fludroxycortide (flurandrenolone, flurandrenolide)=6α-fluoro-11β,16α,17α,21-tetrahydroxypregn-4-ene-3,20-dione cyclic 16α,17α-acetal with acetone, Flunisolide=6α-fluoro-11β,16α,17α,21-tetrahydroxypregna-1,4-diene-3,20-dione cyclic 16α,17α-acetal with acetone, Fluocinolone acetonide=6α,9α-difluoro-11β,16α,17α,21-tetrahydroxypregna-1,4-diene-3,20-dione cyclic 16α,17α-acetal with acetone, Fluocinonide=6α,9α-difluoro-11β,16α,17α,21-tetrahydroxypregna-1,4-diene-3,20-dione cyclic 16α,17α-acetal with acetone, 21-acetate, Halcinonide=9α-fluoro-11β,16α,17α-trihydroxy-21-chloropregn-4-ene-3,20-dione cyclic 16α,17α-acetal with acetone, Triamcinolone acetonide=9α-fluoro-11β,16α,17α,21-tetrahydroxypregna-1,4-diene-3,20-dione cyclic 16α,17α-acetal with acetone, Cortivazol=6,16α-dimethyl-11β,17α,21-trihydroxy-2′-phenyl[3,2-c]pyrazolopregna-4,6-dien-20-one 21-acetate, and RU-28362=6-methyl-11β,17β-dihydroxy-17α-(1-propynyl)androsta-1,4,6-trien-3-one.


SEQUENCE LISTING (45 sequences)

SEQ ID NO: 1 - DNA, 13327 bp, Homo sapiens
  source 1..13327: mol_type = other DNA
gtgtgcagggccagcgcggagcccgagcagccgcggtgaagcgcctgtgctctgccgagactgccgtgcccattgctcgcctcggtcgccgccgctttagccgcctccgggggagcggccgcctattgtctttctccgcggcgaaggtgaagagttgtcccagctcggcccgcgggggagccccgggagccgcacgtgtcctgggtcatgaaacttaatccacagcaagctcccttatatggtgattgtgttgttacagtgctgcttgctgaagaggacaaagctgaagatgatgtagtgttttacttggtatttttgggttccaccctccgtcactgtacaagtactcggaaggtcagttctgatacattggagaccattgctcctggtcatgattgttgtgaaacagtgaaggtgcagctctgtgcttccaaagagggccttcccgtgtttgtggtggctgaagaagactttcatttcgtccaggatgaagcgtatgatgcagctcaattcctagcaaccagtgctggaaatcagcaggctttgaactttacccgttttcttgaccagtcaggacccccatctggggatgtgaattcccttgataagaagttggtgctggcattcaggcacctgaagctgcccacggagtggaatgtattggggacagatcagagtttgcatgatgctggcccgcgagagacattgatgcattttgctgtgcggctgggactgctgaggttgacgtggttcctgttgcagaagccaggtggccgcggagctctcagtatccacaaccaggaaggggcgacgcctgtgagcttggccttggagcgaggctatcacaagctgcaccagcttctaaccgaggagaatgctggagaaccagactcctggagcagtttatcctatgaaataccgtatggagactgttctgtgaggcatcatcgagagttggacatctatacattaacctctgagtctgattcacatcatgaacacccatttcctggagacggttgcactggaccaatttttaaacttatgaacatccaacagcaactaatgaaaacaaacctcaagcagatggacagtcttatgcccttaatgatgacagcacaggatccttccagtgccccagagacagatggccagtttcttccctgtgcaccggagcccacggaccctcagcgactttcttcttctgaagagactgagagcactcagtgctgcccagggagccctgttgcacagactgaaagtccctgtgatttgtcaagcatagttgaggaggagaatacagaccgttcctgtaggaagaaaaataaaggcgtggaaagaaaaggggaagaggtggagccagcacctattgtggactctggaactgtatctgatcaagacagctgccttcagagcttgcctgattgtggagtaaagggcacggaaggcctttcgtcctgtggaaacagaaatgaagaaactggaacaaaatcttctggaatgcccacagaccaggagtccctgagcagtggagatgctgtgcttcagagagacttggtcatggagccaggcacagcccagtattcctctggaggtgaactgggaggcatttcaacaacaaatgtcagtaccccagacactgcaggggaaatggaacatgggctcatgaacccagatgccactgtttggaagaatgtgcttcagggaggggaaagtacaaaggaaagatttgagaactctaatattggcacagctggagcctctgacgtgcacgtcacaagtaagcctgtggataaaatcagtgttccaaactgtgcccctgctgccagttccctggatggtaacaaacctgctgagtcttcacttgcatttagtaatgaagaaacctccactgaaaaaacagcagaaacggaaacttcacgaagtcgtgaggagagtgctgatgctccagtagatcagaattctgtggtgattccagctgctgcaaaagacaagatttcagatggattagaaccttatactctcttagcagcaggcataggtgaggcaatgtcaccctcagatttagcccttcttgggctggaagaagatgtaatgccacaccagaactcagaaacaaattcatctcatgctcaaagccaaaagggcaaatcctcacccatttgttctacaactggagacgataaactttgtgcagactctgcatgtcaacagaacacagtgacttctagtggcgatttggttgcaaaactgtgtgataacatagttagcgagtccgaaagcaccacagcaaggcaacccagctcacaagatccacccgatgcctcccactgtgaagacccacaggctcatacagtcacctctgaccctgtaagggatacccaggaacgtgcggatttttgtcctttcaaagtggtggataacaaaggccaacgaaaagatgtgaaactagataaacctttaacaaatatgcttgaggtggtttcacatccacatccagttgtccctaaaatggagaaagaactggtgccagaccaggcagtaatatcagacagtactttctctctggcaaacagtccaggcagtgaatcagtaaccaaggatgacgcactttcttttgtcccctcccagaaagaaaagggaacagcaactcctgaactacatacagctacagattatagagatggcccagatggaaattcgaatgagcctgatacgcggccactagaagacagggcagtaggcctgtccacatcctccactgctgcagagcttcagcacgggatggggaataccagtctcacaggacttggtggagagcatgagggtcccgcccctccagcaatcccagaagctctgaatatcaaggggaacactgactcttccctgcaaagtgtgggtaaggccactttggctttagattcagttttgactgaagaaggaaaacttctggtggtttcagaaagctctgcagctcaggaacaagataaggataaagcggtgacctgttcctctattaaggaaaatgctctctcttcaggaactttgcaggaagagcagagaacaccacctcctggacaagatactcaacaatttcatgaaaaatcaatctcagctgactgtgccaaggacaaagcacttcagctaagtaattcaccgggtgcatcctctgcctttcttaaggcagaaactgaacataacaaggaagtggccccacaagtctcactgctgactcaaggtggggctgcccagagcctggtgccaccaggagcaagtctggccacagagtcaaggcaggaagccttgggggcagagcacaacagctccgctctgttgccatgtctgttgccagatgggtctgatgggtccgatgctcttaactgcagtcagccttctcctctggatgttggagtgaagaacactcaatcccagggaaaaactagtgcctgtgaggtgagtggagatgtgacggtggatgttacaggggttaatgctctacaaggtatggctgagcccagaagagagaatatatcacacaacacccaagacatcctgattccaaacgtcttgttgagccaagagaagaatgccgttctaggtttgccagtggctctacaggacaaagctgtgactgacccacagggagttggaaccccagagatgatacctcttgattgggagaaagggaagctggagggagcagaccacagctgtaccatgggtgacgctgaggaagcccaaatagacgatgaagcacatcctgtcctactgcagcctgttgccaaggagctccccacagacatggagctctcagcccatgatgatggggccccagctggtgtgagggaagtcatgcgagccccgccttcaggcagggaaaggagcactccctctctaccttgcatggtctctgcccaggacgcacctctgcctaagggggcagacttgatagaggaggctgccagccgtatagtggatgctgtcatcgaacaagtcaaggccgctggagcactgcttactgagggggaggcctgtcacatgtcactgtccagccctgagttgggtcctctcactaaaggactagagagtgcttttacagaaaaagtgagtactttcccacctggggagagcctaccaatgggcagtactcctgaggaagccacggggagccttgcaggatgttttgctggaagggaggagccagagaagatcattttacctgtccaggggcctgagccagcagcagaaatgccagacgtgaaagctgaagatgaagtggattttagagcaagttcaatttctgaagaagtggctgtagggagcatagctgctacactgaagatgaagcaaggcccaatgacccaggcgataaaccgagaaaactggtgtacaatagagccatgccctgatgcagcatctcttctggcttccaagcagagcccagaatgtgagaacttcctggatgttggactgggcagagagtgtacctcaaaacaaggtgtacttaaaagagaatctgggagtgattctgacctctttcactcacccagtgatgacatggacagcatcatcttcccaaagccagaggaagagcatttggcctgtgatatcaccggatccagttcatccaccgatgacacggcttcactggaccgacattcttctcatggcagtgatgtgtctctctcccagattttaaagccaaacaggtcaagagatcggcaaagccttgatggattctacagccatgggatgggagctgagggtcgagaaagtgagagtgagcctgctgacccaggcgacgtggaggaggaggagatggacagtatcactgaagtgcctgcaaactgctctgtcctaaggagctccatgcgctctctttctcccttccggaggcacagctgggggcctgggaaaaatgcagccagcgatgcagaaatgaaccaccggagttcaatgcgagttcttggggatgttgtcaggagacctcccattcataggagaagtttcagtctagaaggcttgacaggaggagctggtgtcggaaacaagccatcctcatctctagaagtaagctctgcaaatgccgaagagctcagacacccattcagtggtgaggaacgggttgactctttggtgtcactttcagaagaggatctggagtcagaccagagagaacataggatgtttgatcagcagatatgtcacagatctaagcagcagggatttaattactgtacatcagccatttcctctccattgacaaaatccatctcattaatgacaatcagccatcctggattggacaattcacggcccttccacagtaccttccacaataccagtgctaatctgactgagagtataacagaagagaactataatttcctgccacatagcccctccaagaaagattctgaatggaagagtggaacaaaagtcagtcgtacattcagctacatcaagaataaaatgtctagcagcaagaagagcaaagaaaaggaaaaagaaaaagataagattaaggagaaggagaaagattctaaagacaaggagaaagataagaagactgtcaacgggcacactttcagttccattcctgttgtgggtcccatcagctgtagccagtgtatgaagcccttcaccaacaaagatgcctatacttgtgcaaattgcagtgcttttgtccacaaaggctgccgagaaagtctagcctcctgtgcaaaggtcaaaatgaagcagcccaaagggagccttcaggcacatgacacatcatcactgcccacggtcattatgagaaacaagccctcacagcccaaggagcgtcctcggtccgcagtcctcctggtggatgaaaccgctaccaccccaatatttgccaatagacgatcccagcagagtgtctcgctctccaaaagtgtctccatacagaacattactggagttggcaatgatgagaacatgtcaaacacctggaaattcctgtctcattcaacagactcactaaataaaatcagcaaggtcaatgagtcaacagaatcacttactgatgagggagtaggtacagacatgaatgaaggacaactactgggagactttgagattgagtccaaacagctggaagcagagtcttggagtcggataatagacagcaagtttctaaaacagcaaaagaaagatgtggtcaaacggcaagaagtaatatatgagttgatgcagacagagtttcatcatgtccgcactctcaagatcatgagtggtgtgtacagccaggggatgatggcggatctgctttttgagcagcagatggtagaaaagctgttcccctgtttggatgagctgatcagtatccatagccaattcttccagaggattctggagcggaagaaggagtctctggtggataaaagtgaaaagaactttctcatcaagaggataggggatgtgcttgtaaatcagttttcaggtgagaatgcagaacgtttaaagaagacatatggcaagttttgtgggcaacataaccagtctgtaaactacttcaaagacctttatgccaaggataagcgttttcaagcctttgtaaagaagaagatgagcagttcagttgttagaaggcttggaattccagagtgcatattgcttgtaactcagcggattaccaagtacccagttttattccaaagaatattgcagtgtaccaaagacaatgaagtggagcaggaagatctagcacagtccttgagcctggtgaaggatgtgattggagctgtagacagcaaagtggcaagttatgaaaagaaagtgcgtctcaatgagatttatacaaagacagatagcaagtcaatcatgaggatgaagagtggtcagatgtttgccaaggaagatttgaaacggaagaagcttgtacgtgatgggagtgtgtttctgaagaatgcagcaggaaggttgaaagaggttcaagcagttcttctcactgacattttagttttccttcaagaaaaagaccagaagtacatctttgcatcattggaccagaagtcaacagtgatctctttaaagaagctgattgtgagagaagtggcacatgaggagaaaggtttattcctgatcagcatggggatgacagatccagagatggtagaagtccatgccagctccaaagaggaacgaaacagctggattcagatcattcaggacacaatcaacaccctgaacagagatgaagatgaaggaattcctagtgagaatgaggaagaaaagaaaatgttggacaccagagcccgagaattaaaagaacaacttcaccagaaggaccaaaaaatcctactcttgttggaagagaaggagatgattttccgggacatggctgagtgcagcacccctctcccagaggattgctccccaacacatagccctagagttctcttccgctccaacacagaagaggctctcaaaggaggacctttaatgaaaagtgcaataaatgaggtggagatccttcagggtttggtgagtggaaatctgggaggcacacttgggccgactgtcagcagccccattgagcaagatgtggtcggtcccgtttccctgccccggagagcagagacctttggaggatttgacagccatcagatgaatgcttcaaaaggaggcgagaaggaagagggagatgatggccaagatcttaggagaacggaatcagatagtggcctaaaaaagggtggaaatgctaacctggtatttatgcttaaaagaaacagtgagcaggttgtccagagcgttgttcatctctacgagctcctcagcgctctgcagggtgtggtgctgcagcaggacagctacattgaggaccagaaactggtgctgagcgagagggcgctcactcgcagcttgtcccgcccgagctccctcattgagcaggagaagcagcgcagcctggagaagcagcgccaggacctggccaacctgcagaagcagcaggcccagtacctcgaggagaagcgcaggcgcgagcgtgagtgggaagctcgtgagagggagctgcgggagcgggaggccctcctggcccagcgcgaggaggaggtgcagcaggggcagcaggacctggaaaaggagcgggaggagctccagcagaagaagggcacataccagtatgacctggagcgactgcgtgctgcccagaaacagcttgagagggaacaggagcagctgcgccgggaggcagagcggctcagccagcggcagacagaacgggacctgtgtcaggtttcccatccacataccaagctgatgaggatcccatcgttcttccccagtcctgaggagcccccctcgccatctgcaccttccatagccaaatcagggtcattggactcagaactttcagtgtccccaaaaaggaacagcatctctcggacacacaaagataaggggccttttcacatactgagttcaaccagccagacaaacaaaggaccagaagggcagagccaggcccctgcgtccacctctgcctctacccgcctgtttgggttaacaaagccaaaggaaaagaaggagaaaaaaaagaagaacaaaaccagccgctctcagcccggtgatggtcccgcgtcagaagtatcagcagagggtgaagagatcttctgctgaccctcttcctctctgctgaggcagctgcctcctgatcctggccagcccacctctcctgctgtccccgcgtgcacaagtctcttacactggacgcccactgctcctcagcgtccagtcctcctgggcggccccaggtcctggacaataagcaacagatgatattgagtgtcgggtggggaaggaggcccagactctgcttcggccatgatttgtgactgcccaggactctcaggttgggctggccctactcaggattacactgaaagtaatggcctcgtaagtacaggtgatggttttggacacgtcaggaattcctaaaggctgaaagagtgtatccaagtaaggtctgaacctccgaatgccttttatttgggggaacacaaaaccaaacagcagatgttttggacttgatctgtgtacgtacatggggacctgtctgcatatacacacggggaatgccagaagaaggcccagtctgcaccaggcgtctggtcaacttagcacaagggcagtgcctggacggacccggagcccccgcatatcagcagttcacccagtactcctcagagactggtttccctctaaacccatcccgggcacataccacccgtgttttgcatgtatttctcatttcattttagggatgacaaacatttgtgaaaccagtgagagaaggcttgatgtgtataaaagacgtgatgtgcaccacctcgatctcggtgtttcaggcactaaagcaacaaaacaacccatagtatctcattctgtcatcagatccagaagaaatatcctggttttccagcatgtttacccacatgttttggccatggataaagtgaagaggcctactcaccattatccctgcagcgtgacaccttttgattgtcactgaccactcagaaggggccacggcctcctggctgtgttcctgagcccccgtcgtgcctctcccagacagcagctgtctggcccttgctgggtgagggcacaccactgccaggggtcagcctcgcacccaggccaggcagaagctgtgctctgaagctaggacagctggctgagaagtgggttcaggcgaagggtgaagccatgtgtagcagttcctgccagtgcagatctggagaggagctggcccggaaggcgtggttgtgaaagcgcccttcttatgttaggaggccttggcaaaattggatttcttcaaaaatacatgtaaaggtctgttgttgaattgtactctgcccctggaagcagatacagatggctgcctgctgctcggctttgcttttgcttttcccaccgtgttttcatctttgttcacttgaggctttccccagctggtgtgtgcaggacagttcatggtaatgttgccctctgaggccccgtacaccagaagggaggccctggaaaattttgtgcttccaacgtggccttcaattcttgcttttttgcccctcggaagcatggggcttttgagcacacttaaaaaaagaaaaatctgtaacttggtgcttattgatgaattgcaagctggccttgcagatggagatatttatctttcagtttatttgaaagaggtctggtttaaaatttgtagcctacatttgttttatttattgtatttgtgtgtttgtgtttgtttttttttaagggtgagccaggtctagcccaacagtctaaactatccagtcaataccgagtgaagtggcagccagcactgttcactctgtgtcttttgaagtgccttgaaggcccagatgaaattttaaagggagggggtccatgtccttccctcccccaccccgcctcattctttaatcaaaggatgtcttctcccttgtttgagaatgaagaaactcgccacctctgacctacctttgcctttttctgtcatggagaatactcacccttcagaaacagaccaaaggccaaaacctgctgatttttctattgaaaatatgtccccttgcaaagaccctaaacaaaaagttaagtttctttctttcacctatttgtacaactccaagttacagctgaatctgtcgtgactttcctgagatctacccggggcttggctgtctgttctgggcactggctccgagttcccctcctgggatttgcaggagggcagtactgaacctgcattcttctccttgtaaatgtaggccgggtgcccctgttctccgggtttggaacaatacgaggttggtgctgatgggatttacttgcgtacgtgctcttcacaaaaacaccgtggatgctgaagttagagcacgtcgccacagagcttgacatcaatgttagagggtctcttactccccgcccagctgtgatgtttcatctgctttggttgttttggtggtcttttttaaaaatagagatttcacatctgcccagaccccactcaaaacgatttggtcaggttctggttggacaagtttaaaatcaaagtagtgcccggaattccctcaaaccacccaacttcatccaggaatacagtctgcagtgcagcaacagaaccgcttaccaagaactgtgcttacatacctttgtcatctctcttccccccttggaagttgtcctcagggggatttgttcctgtcctggggatttacctgggatggtggctgcctgtgcttttgctcatggccttgacagtgctctagttgctggatctaatggcctgtcttggtttctatcacatgagaaggggttgtttttttggggtgactcggactgaattccccatactgtttccacgccgggacaccatgttctccatcaagctaaagaaatcacgtgcctgaaactgtgcttaagttttgggggaaagatggagttcctatccagagcccccagatttccagaatcgagtgagcttcctggaaggagactgcgtcttctctcaattccagtcatctcagtcgttgtcgttaggtgacatgtgcactttaaatgctctcatcggttggcttcattttcaagacaatcaaatgtattgactgtgttttcttcttagaaaatggagagggttaaaaacatgcaaactgccactttcaacctttgccagtattccctctacccccgtgagagctatctggggggaagaatccttaccaaggtttttttggaaaggtacgaatcttaacttttttccccttctgtgtctcagggtaatactattcagagtcgcccctttgctcattttctcccgtatttgttaccttcctgaggcctcagtattagtcgtgagcacaaagttttgagacctttggcgttgtttcttgatgtgggaggggaggtgttagtgcatgcaagggttgaactagatagaccctgccttagtagagggtgggactataaccttagaggccagaacttgatccagaagttgctgtccacagaagtgctttctatttcatcatttttgtttctagggctctttttctgtagccaggtcttcccaaggattttagtatttgcattggagttgaggtttactctaatgatggtggcccagctgtgcccagaggacagccaggcaggccctgggagggagtttagaaagacagtcctggtgaatgggcttcaagtggtcacaaagagggtggctgtgaggtgaccccagacactgcagaacgatgtgcaccctctgcgttttggatgtccttggaatgtgggagcctagaaataaccctgtggatggaattggggcagcggctgctggagatctgtgtgccttgccttccttcagcaggaccgtctaggtgcgcagccacctatggatgcgtcccagccagccccgtcgctctcgtccatcctcagagacaaagaagagggcagggagtttgggcttggttttgaactttcctttcaatgtagcaaagcattcctagttaaccagagccttggaatctactgcctgctggccaggctttaaaatgaaaagtgttttaatgctgccataaaagggaggcgggggggaggaagggaaaataaaggcatctttccaagtactcatctaatttaattgtcaaaagattgataggccatgaattacttctccatctcactaagggttaaaggcgtgcaaccccccactggctgtgtcccctgccaccgaagtgagtgacctgccctacaaccaggtgggaccacctgtgctgcagtccggaggggcttctgcaggaagcactcaccccccacaccttccccggcctgagcttcccctacctttcgtcaccacctgagggcatgagcacaggccatggggcgtgcctggtgagtctgcctgtggttcaggcttagcctgtggtctcctgtgtgctgctgcccgcatgggatgcgcaggggaggcgtggggatccgcaggagggtggttgggatacaccggatacctctgctctcattgcttgtttgcaaatgctctatggacatttgtgtgctaaatcctattaaataaaaaagacgggttaaaacccagatgctgtatattcatttgtaattatgtataaagtgaagcagttttaaactgtaaagatttttttcagtgtgttttctcgaattttgccacaacatactggcttcgtattttatttatctttctttctagttaccagcttcagacccttgtaaagtctccctcagccctttcaaaaaataataaatttcctgtgaagtt

SEQ ID NO: 2 - DNA, 2561 bp, Homo sapiens
  source 1..2561: mol_type = other DNA
gcatccaaccccgggccgcggccaacttctctggactggaccagaagtttctagccggccagttgctacctccctttatctcctccttcccctctggcagcgaggaggctatttccagacacttccacccctctctggccacgtcacccccgcctttaattcataaaggtgcccggcgccggcttcccggacacgtcggcggcggagaggggcccacggcggcggcccggccagagactcggcgcccggagccagcgccccgcacccgcgccccagcgggcagaccccaacccagcatgagcgccgccacccactcgcccatgatgcaggtggcgtccggcaacggtgaccgcgaccctttgccccccggatgggagatcaagatcgacccgcagaccggctggcccttcttcgtggaccacaacagccgcaccactacgtggaacgacccgcgcgtgccctctgagggccccaaggagactccatcctctgccaatggcccttcccgggagggctctaggctgccgcctgctagggaaggccaccctgtgtacccccagctccgaccaggctacattcccattcctgtgctccatgaaggcgctgagaaccggcaggtgcaccctttccatgtctatccccagcctgggatgcagcgattccgaactgaggcggcagcagcggctcctcagaggtcccagtcacctctgcggggcatgccagaaaccactcagccagataaacagtgtggacaggtggcagcggcggcggcagcccagcccccagcctcccacggacctgagcggtcccagtctccagctgcctctgactgctcatcctcatcctcctcggccagcctgccttcctccggcaggagcagcctgggcagtcaccagctcccgcgggggtacatctccattccggtgatacacgagcagaacgttacccggccagcagcccagccctccttccaccaagcccagaagacgcactacccagcgcagcagggggagtaccagacccaccagcctgtgtaccacaagatccagggggatgactgggagccccggcccctgcgggcggcatccccgttcaggtcatctgtccagggtgcatcgagccgggagggctcaccagccaggagcagcacgccactccactccccctcgcccatccgtgtgcacaccgtggtcgacaggcctcagcagcccatgacccatcgagaaactgcacctgtttcccagcctgaaaacaaaccagaaagtaagccaggcccagttggaccagaactccctcctggacacatcccaattcaagtgatccgcaaagaggtggattctaaacctgtttcccagaagcccccacctccctctgagaaggtagaggtgaaagttccccctgctccagttccttgtcctcctcccagccctggcccttctgctgtcccctcttcccccaagagtgtggctacagaagagagggcagcccccagcactgcccctgcagaagctacacctccaaaaccaggagaagccgaggctcccccaaaacatccaggagtgctgaaagtggaagccatcctggagaaggtacaggggctggagcaggctgtagacaactttgaaggcaagaagactgacaaaaagtacctgatgatcgaagagtatttgaccaaagagctgctggccctggattcagtggaccccgagggacgagccgatgtgcgtcaggccaggagagacggtgtcaggaaggttcagaccatcttggaaaaacttgaacagaaagccattgatgtcccaggtcaagtccaggtctatgaactccagcccagcaaccttgaagcagatcagccactgcaggcaatcatggagatgggtgccgtggcagcagacaagggcaagaaaaatgctggaaatgcagaagatccccacacagaaacccagcagccagaagccacagcagcagcgacttcaaaccccagcagcatgacagacacccctggtaacccagcagcaccgtagcctctgccctgtaaaaatcagactcggaaccgatgtgtgctttagggaattttaagttgcatgcatttcagagactttaagtcagttggtttttattagctgcttggtatgcagtaacttgggtggaggcaaaacactaataaaagggctaaaaaggaaaatgatgcttttcttctatattcttactctgtacaaataaagaagttgcttgttgtttgagaagtttaaccccgttgcttgttgttctgcagccctgtctacttgggcacccccaccacctgttagctgtggttgtgcactgtcttttgtagctctggactggaggggtagatggggagtcaattacccatcacataaatatgaaacatttatcagaaatgttgccattttaatgagatgattttcttcatctcataattaaaatacctgactttagagagagtaaaatgtgccaggagccataggaatatctgtatgttggatgactttaatgctacattttaaaaaaagaaaataaagtaataatataactcaaaa

SEQ ID NO: 3 - DNA, 648 bp, Homo sapiens
  source 1..648: mol_type = other DNA
gcgctgcggccgcccgcgcggacccggcgagaggcggcggcgggagcggcggtgatggacgggtccggggagcagcccagaggcggggggcccaccagctctgagcagatcatgaagacaggggcccttttgcttcaggggatgattgccgccgtggacacagactccccccgagaggtctttttccgagtggcagctgacatgttttctgacggcaacttcaactggggccgggttgtcgcccttttctactttgccagcaaactggtgctcaaggccctgtgcaccaaggtgccggaactgatcagaaccatcatgggctggacattggacttcctccgggagcggctgttgggctggatccaagaccagggtggttgggacggcctcctctcctactttgggacgcccacgtggcagaccgtgaccatctttgtggcgggagtgctcaccgcctcactcaccatctggaagaagatgggctgaggcccccagctgccttggactgtgtttttcctccataaattatggcatttttctgggaggggtggggattgggggacgtgggcatttttcttacttttgtaattattggggggtgtggggaagagtggtcttgagggggtaataaacctccttcgggacaca

SEQ ID NO: 4 - DNA, 3306 bp, Homo sapiens
  source 1..3306: mol_type = other DNA
agaactggggttcttagaagtggtgatgcaagaagtttctaggaaaggccggacaccaggttttgagcaaaattttggactgtgaagcaaggcattggtgaagacaaaatggcctcgccggctgacagctgtatccagttcacccgccatgccagtgatgttcttctcaaccttaatcgtctccggagtcgagacatcttgactgatgttgtcattgttgtgagccgtgagcagtttagagcccataaaacggtcctcatggcctgcagtggcctgttctatagcatctttacagaccagttgaaatgcaaccttagtgtgatcaatctagatcctgagatcaaccctgagggattctgcatcctcctggacttcatgtacacatctcggctcaatttgcgggagggcaacatcatggctgtgatggccacggctatgtacctgcagatggagcatgttgtggacacttgccggaagtttattaaggccagtgaagcagagatggtttctgccatcaagcctcctcgtgaagagttcctcaacagccggatgctgatgccccaagacatcatggcctatcggggtcgtgaggtggtggagaacaacctgccactgaggagcgcccctgggtgtgagagcagagcctttgcccccagcctgtacagtggcctgtccacaccgccagcctcttattccatgtacagccacctccctgtcagcagcctcctcttctccgatgaggagtttcgggatgtccggatgcctgtggccaaccccttccccaaggagcgggcactcccatgtgatagtgccaggccagtccctggtgagtacagccggccgactttggaggtgtcccccaatgtgtgccacagcaatatctattcacccaaggaaacaatcccagaagaggcacgaagtgatatgcactacagtgtggctgagggcctcaaacctgctgccccctcagcccgaaatgccccctacttcccttgtgacaaggccagcaaagaagaagagagaccctcctcggaagatgagattgccctgcatttcgagccccccaatgcacccctgaaccggaagggtctggttagtccacagagcccccagaaatctgactgccagcccaactcgcccacagagtcctgcagcagtaagaatgcctgcatcctccaggcttctggctcccctccagccaagagccccactgaccccaaagcctgcaactggaagaaatacaagttcatcgtgctcaacagcctcaaccagaatgccaaaccagaggggcctgagcaggctgagctgggccgcctttccccacgagcctacacggccccacctgcctgccagccacccatggagcctgagaaccttgacctccagtccccaaccaagctgagtgccagcggggaggactccaccatcccacaagccagccggctcaataacatcgttaacaggtccatgacgggctctccccgcagcagcagcgagagccactcaccactctacatgcaccccccgaagtgcacgtcctgcggctctcagtccccacagcatgcagagatgtgcctccacaccgctggccccacgttccctgaggagatgggagagacccagtctgagtactcagattctagctgtgagaacggggccttcttctgcaatgagtgtgactgccgcttctctgaggaggcctcactcaagaggcacacgctgcagacccacagtgacaaaccctacaagtgtgaccgctgccaggcctccttccgctacaagggcaacctcgccagccacaagaccgtccataccggtgagaaaccctatcgttgcaacatctgtggggcccagttcaaccggccagccaacctgaaaacccacactcgaattcactctggagagaagccctacaaatgcgaaacctgcggagccagatttgtacaggtggcccacctccgtgcccatgtgcttatccacactggtgagaagccctatccctgtgaaatctgtggcacccgtttccggcaccttcagactctgaagagccacctgcgaatccacacaggagagaaaccttaccattgtgagaagtgtaacctgcatttccgtcacaaaagccagctgcgacttcacttgcgccagaagcatggcgccatcaccaacaccaaggtgcaataccgcgtgtcagccactgacctgcctccggagctccccaaagcctgctgaagcatggagtgttgatgctttcgtctccagccccttctcagaatctacccaaaggatactgtaacactttacaatgttcatcccatgatgtagtgcctctttcatccactagtgcaaatcatagctgggggttgggggtggtgggggtcggggcctgggggactgggagccgcagcagctccccctcccccactgccataaaacattaagaaaatcatattgcttcttctcctatgtgtaaggtgaaccatgtcagcaaaaagcaaaatcattttatatgtcaaagcaggggagtatgcaaaagttctgacttgactttagtctgcaaaatgaggaatgtatatgttttgtgggaacagatgtttcttttgtatgtaaatgtgcattcttttaaaagacaagacttcagtatgttgtcaaagagagggctttaatttttttaaccaaaggtgaaggaatatatggcagagttgtaaatatataaatatatatatatataaaataaatatatataaacctaaaaaagatatattaaaaatataaaactgcgttaaaggctcgattttgtatctgcaggcagacacggatctgagaatctttattgagaaagagcacttaagagaatattttaagtattgcatctgtataagtaagaaaatattttgtctaaaatgcctcagtgtatttgtatttttttgcaagtgaaggtttacaatttacaaagtgtgtattaaaaaaaacaaaaagaacaaaaaaatctgcagaaggaaaaatgtgtaattttgttctagttttcagtttgtatatacccgtacaacgtgtcctcacggtgccttttttcacggaagttttcaatgatgggcgagcgtgcaccatccctttttgaagtgtaggcagacacagggacttgaagttgttactaactaaactctctttgggaatgtttgtctcatcccattctgcgtcatgcttgtgttataactactccggagacagggtttggctgtgtctaaactgcattaccgcgttgtaaaatatagctgtacaaatataagaataaaatgttgaaaagtcaaa

SEQ ID NO: 5 - DNA, 6877 bp, Homo sapiens
  source 1..6877: mol_type = other DNA
ggcacggaaaaggccaggcgacaggtgtcgcttgaaaagactgggcttgtccttgctggtgcatgcgtcgtcggcctctgggcagcaggtttacaaaggaggaaaacgacttcttctagatttttttttcagtttcttctataaatcaaaacatctcaaaatggagacctaaaatccttaaagggacttagtctaatctcgggaggtagttttgtgcatgggtaaacaaattaagtattaactggtgttttactatccaaagaatgctaattttataaacatgatcgagttatataaggtataccataatgagtttgattttgaatttgatttgtggaaataaaggaaaagtgattctagctggggcatattgttaaagcatttttttcagagttggccaggcagtctcctactggcacattctcccattatgtagaatagaaatagtacctgtgtttgggaaagattttaaaatgagtgacagttatttggaacaaagagctaataatcaatccactgcaaattaaagaaacatgcagatgaaagttttgacacattaaaatacttctacagtgacaaagaaaaatcaagaacaaagctttttgatatgtgcaacaaatttagaggaagtaaaaagataaatgtgatgattggtcaagaaattatccagttatttacaaggccactgatattttaaacgtccaaaagtttgtttaaatgggctgttaccgctgagaatgatgaggatgagaatgatggttgaaggttacattttaggaaatgaagaaacttagaaaattaatataaagacagtgatgaatacaaagaagatttttataacaatgtgtaaaatttttggccagggaaaggaatattgaagttagatacaattacttacctttgagggaaataattgttggtaatgagatgtgatgtttctcctgccacctggaaacaaagcattgaagtctgcagttgaaaagcccaacgtctgtgagatccaggaaaccatgcttgcaaaccactggtaaaaaaaaaaaaaaaaaaaaaaaaaagccacagtgacttgcttattggtcattgctagtattatcgactcagaacctctttactaatggctagtaaatcataattgagaaattctgaattttgacaaggtctctgctgttgaaatggtaaatttattattttttttgtcatgataaattctggttcaaggtatgctatccatgaaataatttctgaccaaaactaaattgatgcaatttgattatccatcttagcctacagatggcatctggtaacttttgactgttttaaaaaataaatccactatcagagtagatttgatgttggcttcagaaacatttagaaaaacaaaagttcaaaaatgttttcaggaggtgataagttgaataactctacaatgttagttctttgagggggacaaaaaatttaaaatctttgaaaggtcttattttacagccatatctaaattatcttaagaaaatttttaacaaagggaatgaaatatatatcatgattctgtttttccaaaagtaacctgaatatagcaatgaagttcagttttgttattggtagtttgggcagagtctctttttgcagcacctgttgtctaccataattacagaggacatttccatgttctagccaagtatactattagaataaaaaaacttaacattgagttgcttcaacagcatgaaactgagtccaaaagaccaaatgaacaaacacattaatctctgattatttattttaaatagaatatttaattgtgtaagatctaatagtatcattatacttaagcaatcatattcctgatgatctatgggaaataactattatttaattaatattgaaaccaggttttaagatgtgttagccagtcctgttactagtaaatctctttatttggagagaaattttagattgttttgttctccttattagaaggattgtagaaagaaaaaaatgactaattggagaaaaattggggatatatcatatttcactgaattcaaaatgtcttcagttgtaaatcttaccattattttacgtacctctaagaaataaaagtgcttctaattaaaatatgatgtcattaattatgaaatacttcttgataacagaagttttaaaatagccatcttagaatcagtgaaatatggtaatgtattattttcctcctttgagttaggtcttgtgcttttttttcctggccactaaatttcacaatttccaaaaagcaaaataaacatattctgaatatttttgctgtgaaacacttgacagcagagctttccaccatgaaaagaagcttcatgagtcacacattacatctttgggttgattgaatgccactgaaacattctagtagcctggagaagttgacctacctgtggagatgcctgccattaaatggcatcctgatggcttaatacacatcactcttctgtgaagggttttaattttcaacacagcttactctgtagcatcatgtttacattgtatgtataaagattatacaaaggtgcaattgtgtatttcttccttaaaatgtatcagtataggatttagaatctccatgttgaaactctaaatgcatagaaataaaaataataaaaaatttttcattttggcttttcagcctagtattaaaactgataaaagcaaagccatgcacaaaactacctccctagagaaaggctagtcccttttcttccccattcatttcattatgaacatagtagaaaacagcatattcttatcaaatttgatgaaaagcgccaacacgtttgaactgaaatacgacttgtcatgtgaactgtaccgaatgtctacgtattccacttttcctgctggggttcctgtctcagaaaggagtcttgctcgtgctggtttctattacactggtgtgaatgacaaggtcaaatgcttctgttgtggcctgatgctggataactggaaaagaggagacagtcctactgaaaagcataaaaagttgtatcctagctgcagattcgttcagagtctaaattccgttaacaacttggaagctacctctcagcctacttttccttcttcagtaacaaattccacacactcattacttccgggtacagaaaacagtggatatttccgtggctcttattcaaactctccatcaaatcctgtaaactccagagcaaatcaagatttttctgccttgatgagaagttcctaccactgtgcaatgaataacgaaaatgccagattacttacttttcagacatggccattgacttttctgtcgccaacagatctggcaaaagcaggcttttactacataggacctggagacagagtggcttgctttgcctgtggtggaaaattgagcaattgggaaccgaaggataatgctatgtcagaacacctgagacattttcccaaatgcccatttatagaaaatcagcttcaagacacttcaagatacacagtttctaatctgagcatgcagacacatgcagcccgctttaaaacattctttaactggccctctagtgttctagttaatcctgagcagcttgcaagtgcgggtttttattatgtgggtaacagtgatgatgtcaaatgcttttgctgtgatggtggactcaggtgttgggaatctggagatgatccatgggttcaacatgccaagtggtttccaaggtgtgagtacttgataagaattaaaggacaggagttcatccgtcaagttcaagccagttaccctcatctacttgaacagctgctatccacatcagacagcccaggagatgaaaatgcagagtcatcaattatccattttgaacctggagaagaccattcagaagatgcaatcatgatgaatactcctgtgattaatgctgccgtggaaatgggctttagtagaagcctggtaaaacagacagttcagagaaaaatcctagcaactggagagaattatagactagtcaatgatcttgtgttagacttactcaatgcagaagatgaaataagggaagaggagagagaaagagcaactgaggaaaaagaatcaaatgatttattattaatccggaagaatagaatggcactttttcaacatttgacttgtgtaattccaatcctggatagtctactaactgccggaattattaatgaacaagaacatgatgttattaaacagaagacacagacgtctttacaagcaagagaactgattgatacgattttagtaaaaggaaatattgcagccactgtattcagaaactctctgcaagaagctgaagctgtgttatatgagcatttatttgtgcaacaggacataaaatatattcccacagaagatgtttcagatctaccagtggaagaacaattgcggagactacaagaagaaagaacatgtaaagtgtgtatggacaaagaagtgtccatagtgtttattccttgtggtcatctagtagtatgcaaagattgtgctccttctttaagaaagtgtcctatttgtaggagtacaatcaagggtacagttcgtacatttctttcatgaagaagaaccaaaacatcgtctaaactttagaattaatttattaaatgtattataactttaacttttatcctaatttggtttccttaaaatttttatttatttacaactcaaaaaacattgttttgtgtaacatatttatatatgtatctaaaccatatgaacatatattttttagaaactaagagaatgataggcttttgttcttatgaacgaaaaagaggtagcactacaaacacaatattcaatcaaaatttcagcattattgaaattgtaagtgaagtaaaacttaagatatttgagttaacctttaagaattttaaatattttggcattgtactaataccgggaacatgaagccaggtgtggtggtatgtgcctgtagtcccaggctgaggcaagagaattacttgagcccaggagtttgaatccatcctgggcagcatactgagaccctgcctttaaaaacaaacagaacaaaaacaaaacaccagggacacatttctctgtcttttttgatcagtgtcctatacatcgaaggtgtgcatatatgttgaatgacattttagggacatggtgtttttataaagaattctgtgagaaaaaatttaataaagcaacaaaaattactcttattcttcattgctttatttcaatgacattggatagtttagtcactcccagactctttccataccttcttaaagcctctcaaatattgaactacagtttatactccttcccataagatgcttcttcattgacacttgtagaacacggggtcaacacatcataaaatctattatggaatgcctgagacaagaatcaaacagtccctttagtaagtttgtttattcacttctctattgattcattcaagaagtctcatgccagccccacctattggaagaaggtctgagttttattcttatctctttggtattaattctgaaacttagaaagtacactggttagcaatgcttgggaccaacaggttgttctggtaaataaatctgtttcatattgtcagtgcaacaaaatgtccccctctgcattatgttattggtactcaacacgtccgagtcataactctgtcctttgcttcttatagaggtattaggtcttcaagagcagaagtaagactgtaatagggaatactcaggggaaggcaggcaaaggctagtcatctaaaccagttctagatgtctgtataggggcagatggctctgtaagggcagaagggaaagaccccttcataagggtcacagctgacaatcctataacaaaagacaggttaacaagagaaaaacttaacaaatttatttaatcacagatttacatcaccggggagccttcgtaatgaagatccaaaattacaggggaaactgtgcatttttatgcttaggtttgataatgaatggacagccctgaagaatagtgattggaaaaaaaggatatgatctaatgggaatagacacaggttggggacccagcaaggcctgtctgttcagattattcttggtctctgtgcagcattccttcctcctggatatagggcagggcctgtatgggatggggatattataacctgctatcaagcaaggtaggtcagagaatttatttatggccagctcttacatagttaggtgaggaaagattagagtactatctttaagatgtaagtctggcattgtggaaagatggttccagtttctatgacctaccttggggaagaggaattcaagtttctgtggcttgccttcagggagaatgaggctgagacaggagggcaggataacatcagagaaaaactttgcttctgaggccttcactttgggttttctgagccccaacatctgctagtgttgtaaagagaacaattagggaccaagtgaggggaggaaagaatccatctctgcattctgatgctgggagacttatttccttgaaatgcaattgattttgcctctgctaagaggctctgctggctacccatgtactagccagtgtcctgcatgggtgctaggctgaattatttgtaattgtgcttaggtgatttgtaactcaggtatagggtatttaaatagtaggcaccctttttgcaccatgtgtttttttttttatctagttcttgtatactacagataatatttgaactttgtcatctcactgtaaaacttttgttcatttctcattatggtaataaatagctattataaccaacccatttattcaaatatgttatttccctaagtgttattttgacattttgttttggaaaaaataaatcaccatagataataa

SEQ ID NO: 6 - DNA, 3054 bp, Homo sapiens
  source 1..3054: mol_type = other DNA
gatcagcgtagccagttgttgcctgtctgaacctctgccagtcctggagactggtgccctgagctccaaccagcgggcctcatcctacaccctcaccaccgcaacttctcacccgagcaagaagcagctcccagagagaaagaacgttcccacctgcctagccatgggagaggacgctgcacaggccgaaaagttccagcaccctgggtctgacatgcggcaggaaaagccctcgagccccagcccgatgccttcctccacaccaagccccagcctgaacctagggaacacagaggaggccatccgggacaactcacaggtgaacgcagtcacggtgctcacgctcctggacaagctggtgaacatgctagacgctgtgcaggagaaccagcacaagatggagcagcgacagatcagtttggagggctccgtgaagggcatccagaatgacctcaccaagctctccaagtaccaggcctccaccagcaacacggtgagcaagctgctggagaagtcccgcaaggtcagcgcccacacgcgcgcggtcaaagagcgcatggataggcagtgcgcacaggtgaagcggctggagaacaaccacgcccagctcctccgacgcaaccatttcaaagtgctcatcttccaggaggaaaatgagatccctgccagcgtgtttgtgaaacagcccgtttccggtgccgtggaagggaaggaggagcttccggatgaaaacaaatccctggaggaaaccctgcacaccgtggacctctcctcagatgatgatttgccccacgatgaggaggccctggaagacagtgccgaggaaaaggtggaagaaagtagggcagagaaaataaaaagatccagcctgaagaaagtggatagcctcaagaaagcattttctcgccagaacatcgagaaaaagatgaacaagctggggacaaagatcgtatctgtagagaggagagagaagattaagaaatctctcacgtcaaatcaccagaaaatatcctcaggaaaaagctcccccttcaaggtttctcccctcactttcgggcggaagaaagtccgagagggagaaagccatgcagaaaatgagaccaagtcagaagacctgcctagcagtgagcagatgccaaatgaccaggaagaggagtcctttgcagagggtcattccgaagcgtccctcgccagcgctctggtggaaggggaaattgcagaggaggctgctgagaaggcgacctccagggggagtaactcggggatggacagcaacatcgacttgactattgtggaagatgaagaggaggagtcagtggccctggaacaggcacagaaggtacgctatgagggtagctacgcgctaacatccgaggaggcggagcgctccgatggggaccccgtgcagcccgccgtgctccaggtgcaccagacctcctgagcttagagccaccgtgccatcctgtgctgtgctcaagcgggcagccagggctgaagaacaaactcttgcacatctccagcacgactcacccactcctgcgttcctgtccaggcagtaatcattgaccatatagtcatagtaagacacacgagaccaggctttaccatgaaagcgacctgtcacggactccacttttaatttgctcttaggttctatctctgtagaatgtctccaagattgaagaagaaactgagcagttgaaaaatgctaatctctttgacttagtcagaaaaaaacagaggataattaagatactagtcatgaaaagtgattcattcttttttgtcattccataagcttgctgaatagtgtaccggtaatatattgtatttccaccgtactctgtgaatctaattattattctttaagtgttgatatataatatacataaatatgtaagctaaacatataactatatgttttaagaagaaaacatctacgaaaggtaaaaagagatgatcagttggttgtttacttgctagaaaccattgttttattgcaaacgaaggaaaaatgaagagattataaaagtcagctaatgaagtaagatacgtagtaaagtcaggactattcaaaaagtaagaaagaaaatttggaaaatgagagaaacaggaaacaaagaatgccgaaaagaatgaaaacagagaaaaaatgtatgtgcttgaaagtaaaatacttacaatagtagcttaactatttcactctttaaataaaaatactaaagaagttcgtatatcctggaataacatgtcatcttcaaaatatttttattttctaatatttttaataataaacattttatagtgttaaagctgtatttttcttaataaataaaggacattacaaatatttctttaaggaagttcaaaaggatgttgtcttgttaacactggaacgaatataaatcttcaaaatacaattaatatatactgttttgtggaaacttagctcatactattctttaatgctcttcaaatttaaactaacccttacattccatgtgatgtgatgtaggcttataagatgctaaaatctataggttttggaagtgaaagattctattttttctttttttgtacataatgggaatttcattccagattatattttatttacatattaatttcacagaatattaatatttcttaacttcttaaagcatgctagcgttttatgtatatgtacacatatattcagacagggtaattttatctgctgcctaacattgtactaaaatattgctttatctgtttttaattacaaaatgctaatgatttcttaaattatagtttaaagacaattggcccaggaagcaaatcccctgcctttagtatgaaccactataagtaaccttacaaatagagttaatccaagacaatattaacaaactgtgctttgtctttaataaaagggataggattaacaaacatattgatggcataacctattcagctatgtccttatttttgcaataatgtaacctcaaatattggattgttgaaccaacaatggctgtgttaaagaagattaaataaaccgaaaaataaaaagca

SEQ ID NO: 7 - DNA, 1943 bp, Homo sapiens
  source 1..1943: mol_type = other DNA
agtgcgctgtgctcgaggggtgccggccaggcctgagcgagcgagctagccagcaggcatcgagggggcgcggctgccgtccggacgagacaggcgaacccgacgcagaagagtccaccaccggacagccaggtagccgccgcgtccctcgcacacgcagagtcgggcggcgcggggtctcccttgcgcccggcctccgccctctcctcctctcctttccccttcttctcgctgtcctctcctctctcgctgcccgcgtttgcgcagccccgggccatgtccgacgcgtccctccgcagcacatccacgatggagcgtcttgtcgcccgtgggaccttcccagtactagtgcgcaccagcgcctgccgcagcctcttcgggccggtggaccacgaggagctgagccgcgagctgcaggcccgcctggccgagctgaacgccgaggaccagaaccgctgggattacgacttccagcaggacatgccgctgcggggccctggacgcctgcagtggaccgaagtggacagcgactcggtgcccgcgttctaccgcgagacggtgcaggtggggcgctgccgcctgctgctggcgccgcggcccgtcgcggtcgcggtggctgtcagcccgcccctcgagccggccgctgagtccctcgacggcctcgaggaggcgccggagcagctgcctagtgtcccggtcccggccccggcgtccaccccgcccccagtcccggtcctggctccagccccggccccggctccggctccggtcgcggctccggtcgcggctccggtcgcggtcgcggtcctggccccggccccggccccggctccggctccggctccggccccggctccagtcgcggccccggccccagccccggccccggccccggccccggcccccgccccggccccggccccggacgcggcgcctcaagagagcgccgagcagggcgcgaaccaggggcagcgcggccaggagcctctcgctgaccagctgcactcggggatttcgggacgtcccgcggccggcaccgcggccgccagcgccaacggcgcggcgatcaagaagctgtccgggcctctgatctccgatttcttcgccaagcgcaagagatcagcgcctgagaagtcgtcgggcgatgtccccgcgccgtgtccctctccaagcgccgcccctggcgtgggctcggtggagcagaccccgcgcaagaggctgcggtgagccaatttagagcccaaagagccccgagggaacctgccggggcagcggacgttggaagggcgctgggcctcggctgggaccgttcatgtagcagcaaccggcggcggctgccgcagagcagcgttcggttttgtttttaaattttgaaaactgtgcaatgtattaataacgtctttttatatctaaatgtattctgcacgagaaggtacactggtcccaaggtgtaaagctttaagagtcatttatataaaatgtttaatctctgctgaaactcagtgcaaaaaaaagaaaaaagaaaaaaaaaaggaaaaaataaaaaaaccatgtatatttgtacaaaaagtttttaaagttatactaacttatattttctatttatgtccaggcgtggaccgctctgccacgcactagctcggttattggttatgccaaaggcactctccatctcccacatctggttattgacaagtgtaactttattttcatcgcggactctggggaagggggtcactcacaagctgtagctgccatacatgcccatctagcttgcagtctcttcgcgctttcgctgtctctcttattatgactgtgtttatctgaaacttgaagacaagtctgttaaaatggttcctgagccgtctgtaccactgccccggcccctcgtccgccgggttctaaataaagaggccgaaaaatgctgcaaaaaaaaaaaaaaa

SEQ ID NO: 8 - DNA, 1252 bp, Homo sapiens
  source 1..1252: mol_type = other DNA
aggtgacagcctcgcttggacgcagagcccggcccgacgccgccatgagcgccgcgctcttcagcctggacggcccggcgcgcggcgcgccctggcctgcggagcctgcgcccttctacgaaccgggccgggcgggcaagccgggccgcggggccgagccaggggccctaggcgagccaggcgccgccgcccccgccatgtacgacgacgagagcgccatcgacttcagcgcctacatcgactccatggccgccgtgcccaccctggagctgtgccacgacgagctcttcgccgacctcttcaacagcaatcacaaggcgggcggcgcggggcccctggagcttcttcccggcggccccgcgcgccccttgggcccgggccctgccgctccccgcctgctcaagcgcgagcccgactggggcgacggcgacgcgcccggctcgctgttgcccgcgcaggtggccgcgtgcgcacagaccgtggtgagcttggcggccgcagggcagcccaccccgcccacgtcgccggagccgccgcgcagcagccccaggcagacccccgcgcccggccccgcccgggagaagagcgccggcaagaggggcccggaccgcggcagccccgagtaccggcagcggcgcgagcgcaacaacatcgccgtgcgcaagagccgcgacaaggccaagcggcgcaaccaggagatgcagcagaagttggtggagctgtcggctgagaacgagaagctgcaccagcgcgtggagcagctcacgcgggacctggccggcctccggcagttcttcaagcagctgcccagcccgcccttcctgccggccgccgggacagcagactgccggtaacgcgcggccggggcgggagagactcagcaacgacccatacctcagacccgacggcccggagcggagcgcgccctgccctggcgcagccagagccgccgggtgcccgctgcagtttcttgggacataggagcgcaaagaagctacagcctggacttaccaccactaaactgcgagagaagctaaacgtgtttattttcccttaaattatttttgtaatggtagctttttctacatcttactcctgttgatgcagctaaggtacatttgtaaaaagaaaaaaaaccagacttttcagacaaaccctttgtattgtagataagaggaaaagactgagcatgctcacttttttatattaatttttacagtatttgtaagaataaagcagcatttgaaatcgc

SEQ ID NO: 9 - DNA, 1985 bp, Homo sapiens
  source 1..1985: mol_type = other DNA
gccaccgctcctgccactgcagtgctcgagccccgtgcaggggagcttgcgggaggatcgaccgacagacggacgcacgccgaggcactgcgcccccagccccgcgccggtgccaccgcagcccgaccccggccgccagtccagccgcccctcgcccggtgcctaggtgcccggccccacaccgccagctgctcggcgcccgggtccgccatgcgctccgccgctgtcctggctcttctgctctgcgccgggcaagtcactgcgctccctgtgaacagccctatgaataaaggggataccgaggtgatgaaatgcatcgttgaggtcatctccgacacactttccaagcccagccccatgcctgtcagccaggaatgttttgagacactccgaggagatgaacggatcctttccattctgagacatcagaatttactgaaggagctccaagacctcgctctccaaggcgccaaggagagggcacatcagcagaagaaacacagcggttttgaagatgaactctcagaggttcttgagaaccagagcagccaggccgagctgaaagaggcggtggaagagccatcatccaaggatgttatggagaaaagagaggattccaaggaggcagagaaaagtggtgaagccacagacggagccaggccccaggccctcccggagcccatgcaggagtccaaggctgaggggaacaatcaggcccctggggaggaagaggaggaggaggaggaggccaccaacacccaccctccagccagcctccccagccagaaatacccaggcccacaggccgagggggacagtgagggcctctctcagggtctggtggacagagagaagggcctgagtgcagagccagggtggcaggcaaagagagaagaggaggaggaggaggaggaggaggctgaggctggagaggaggctgtccccgaggaagaaggccccactgtagtgctgaacccccacccgagccttggctacaaggagatccggaaaggcgagagtcggtcggaggctctggctgtggatggagctgggaagcctggggctgaggaggctcaggaccccgaagggaagggagaacaggagcactcccagcagaaagaggaggaggaggagatggcagtggtcccgcaaggcctcttccggggtgggaagagcggagagctggagcaggaggaggagcggctctccaaggagtgggaggactccaaacgctggagcaagatggaccagctggccaaggagctgacggctgagaagcggctggaggggcaggaggaggaggaggacaaccgggacagttccatgaagctctccttccgggcccgggcctacggcttcaggggccctgggccgcagctgcgacgaggctggaggccatcctcccgggaggacagccttgaggcgggcctgcccctccaggtccgaggctaccccgaggagaagaaagaggaggagggcagcgcaaaccgcagaccagaggaccaggagctggagagcctgtcggccattgaagcagagctggagaaagtggcccaccagctgcaggcactacggcggggctgagacaccggctggcagggctggccccagggcaccctgtggccctggctctgctgtccccttggcaggtcctggccagatggcccggatgctgcttccggtagggaggcagcctccagcctgcccaagcccaggccaccctatcgccccctacgcgccttgtctcctactcctgactcctacctgccctggaacatcctttgcagggcagccccacaactttaaacattgacgattccttctctgaacacaggcagctttctagaagtttcccttcctccatcctatccactgggcacaactgcaataacttctgaccttttggtgaaagctgagaactcctgactgtaacatattctgtatgaactttatctaaagaaaaataaatctgttctgggctcttt

SEQ ID NO: 10 - DNA, 1298 bp, Homo sapiens
  source 1..1298: mol_type = other DNA
agaatgttcttttggccactgtgaagcctcaggaaggggctcggattgctcaaggacccatgggagagaggaggctttgactgggctgcctgcctgtgaggtctctggactagaggtccaacgcagtccagctgacaaggatggaatacgccatgaagtcccttagccttctctaccccaagtccctctccaggcatgtgtcagtgcgtacctctgtggtgacccagcagctgctgtcggagcccagccccaaggcccccagggcccggccctgccgcgtaagcacggcggatcgaagcgtgaggaagggcatcatggcttacagtcttgaggacctcctcctcaaggtccgggacactctgatgctggcagacaagcccttcttcctggtgctggaggaagatggcacaactgtagagacagaagagtacttccaagccctggcaggggatacagtgttcatggtcctccagaaggggcagaaatggcagcccccatcagaacaggggacaaggcacccactgtccctctcccataagcctgccaagaagattgatgtggcccgtgtaacgtttgatctgtacaagctgaacccacaggacttcattggctgcctgaacgtgaaggcgactttttatgatacatactccctttcctatgatctgcactgctgtggggccaagcgcatcatgaaggaagctttccgctgggccctcttcagcatgcaggccacaggccacgtactgcttggcacctcctgttacctgcagcagctcctcgatgctacggaggaagggcagccccccaagggcaaggcctcatcccttatcccgacctgtctgaagatactgcagtgaaagcccaagtccttggaagctttccccagtgaaggactgactgggggcctcacgcttaactggtagtgcccacaagcctggcagctgtagagccgcgaacctccccacacctccctcaccgcgcaggaccctgagtgaggaggaggagctggaaacctggggtgggttggccaaaggagaacctcaagctcctggcctgatccagctccttcctgcccaaggcagcttagcccatccagactggtcctgaagtctgtccctccattggcatgaagtctgccccttagcaatctggcctcgcaggctgtactttcatggtgctctctaccttctggcccccatcccggaacattcctgagtgaattcgcaagcgcactagcatgtgatattagggagtttgcaataaattattgaggctgatgtaaaaaaaaaaaaaaaaaa

SEQ ID NO: 11 - DNA, 1560 bp, Homo sapiens
  source 1..1560: mol_type = other DNA
ctccataaggcacaaactttcagagacagcagagcacacaagcttctaggacaagagccaggaagaaaccaccggaaggaaccatctcactgtgtgtaaacatgacttccaagctggccgtggctctcttggcagccttcctgatttctgcagctctgtgtgaaggtgcagttttgccaaggagtgctaaagaacttagatgtcagtgcataaagacatactccaaacctttccaccccaaatttatcaaagaactgagagtgattgagagtggaccacactgcgccaacacagaaattattgtaaagctttctgatggaagagagctctgtctggaccccaaggaaaactgggtgcagagggttgtggagaagtttttgaagagggctgagaattcataaaaaaattcattctctgtggtatccaagaatcagtgaagatgccagtgaaacttcaagcaaatctacttcaacacttcatgtattgtgtgggtctgttgtagggttgccagatgcaatacaagattcctggttaaatttgaatttcagtaaacaatgaatagtttttcattgtaccatgaaatatccagaacatacttatatgtaaagtattatttatttgaatctacaaaaaacaacaaataatttttaaatataaggattttcctagatattgcacgggagaatatacaaatagcaaaattgggccaagggccaagagaatatccgaactttaatttcaggaattgaatgggtttgctagaatgtgatatttgaagcatcacataaaaatgatgggacaataaattttgccataaagtcaaatttagctggaaatcctggatttttttctgttaaatctggcaaccctagtctgctagccaggatccacaagtccttgttccactgtgccttggtttctcctttatttctaagtggaaaaagtattagccaccatcttacctcacagtgatgttgtgaggacatgtggaagcactttaagttttttcatcataacataaattattttcaagtgtaacttattaacctatttattatttatgtatttatttaagcatcaaatatttgtgcaagaatttggaaaaatagaagatgaatcattgattgaatagttataaagatgttatagtaaatttattttattttagatattaaatgatgttttattagataaatttcaatcagggtttttagattaaacaaacaaacaattgggtacccagttaaattttcatttcagatatacaacaaataattttttagtataagtacattattgtttatctgaaattttaattgaactaacaatcctagtttgatactcccagtcttgtcattgccagctgtgttggtagtgctgtgttgaattacggaataatgagttagaactattaaaacagccaaaactccacagtcaatattagtaatttcttgctggttgaaacttgtttattatgtacaaatagattcttataatattatttaaatgactgcatttttaaatacaaggctttatatttttaactttaaaaaaaaccgg

SEQ ID NO: 12 - DNA, 1744 bp, Homo sapiens
  source 1..1744: mol_type = other DNA
gcagcaggccaagggggaggtgcgagcgtggacctgggacgggtctgggcggctctcggtggttggcacgggttcgcacacccattcaagcggcaggacgcacttgtcttagcagttctcgctgaccgcgctagctgcggcttctacgctccggcactctgagttcatcagcaaacgccctggcgtctgtcctcaccatgcctagcctttgggaccgcttctcgtcgtcgtccacctcctcttcgccctcgtccttgccccgaactcccaccccagatcggccgccgcgctcagcctgggggtcggcgacccgggaggaggggtttgaccgctccacgagcctggagagctcggactgcgagtccctggacagcagcaacagtggcttcgggccggaggaagacacggcttacctggatggggtgtcgttgcccgacttcgagctgctcagtgaccctgaggatgaacacttgtgtgccaacctgatgcagctgctgcaggagagcctggcccaggcgcggctgggctctcgacgccctgcgcgcctgctgatgcctagccagttggtaagccaggtgggcaaagaactactgcgcctggcctacagcgagccgtgcggcctgcggggggcgctgctggacgtctgcgtggagcagggcaagagctgccacagcgtgggccagctggcactcgaccccagcctggtgcccaccttccagctgaccctcgtgctgcgcctggactcacgactctggcccaagatccaggggctgtttagctccgccaactctcccttcctccctggcttcagccagtccctgacgctgagcactggcttccgagtcatcaagaagaagctgtacagctcggaacagctgctcattgaggagtgttgaacttcaacctgagggggccgacagtgccctccaagacagagacgactgaacttttggggtggagactagaggcaggagctgagggactgattcctgtggttggaaaactgaggcagccacctaaggtggaggtgggggaatagtgtttcccaggaagctcattgagttgtgtgcgggtggctgtgcattggggacacatacccctcagtactgtagcatgaaacaaaggcttaggggccaacaaggcttccagctggatgtgtgtgtagcatgtaccttattatttttgttactgacagttaacagtggtgtgacatccagagagcagctgggctgctcccgccccagcccggcccagggtgaaggaagaggcacgtgctcctcagagcagccggagggaggggggaggtcggaggtcgtggaggtggtttgtgtatcttactggtctgaagggaccaagtgtgtttgttgtttgttttgtatcttgtttttctgatcggagcatcactactgacctgttgtaggcagctatcttacagacgcatgaatgtaagagtaggaaggggtgggtgtcagggatcacttgggatctttgacacttgaaaaattacacctggcagctgcgtttaagccttcccccatcgtgtactgcagagttgagctggcaggggaggggctgagagggtgggggctggaacccctccccgggaggagtgccatctgggtcttccatctagaactgtttacatgaagataagatactcactgttcatgaatacacttgatgttcaagtattaagacctatgcaatattttttacttttctaataaacatgtttgttaaaaca

SEQ ID NO: 13 - DNA, 7459 bp, Homo sapiens
  source 1..7459: mol_type = other DNA
agtcactgccgcggcgccttgagtctccgggccgccttgccatggctgcccgtggtgtcatcgctccagttggcgagagtttgcgctacgctgagtacttgcagccctcggccaaacggccagacgccgacgtcgaccagcagagactggtaagaagtttgatagctgtaggactgggtgttgcagctcttgcatttgcaggtcgctacgcatttcggatctggaaacctctagaacaagttatcacagaaactgcaaagaagatttcaactcctagcttttcatcctactataaaggaggatttgaacagaaaatgagtaggcgagaagctggtcttattttaggtgtaagcccatctgctggcaaggctaagattagaacagctcataggagagtcatgattttgaatcacccagataaaggtggatctccttacgtagcagccaaaataaatgaagcaaaagacttgctagaaacaaccaccaaacattgatgcttaaggaccacactgaaggaaaaaaaaagaggggacttcgaaaaaaaaaaaagccctgcaaaatattctaaaacatggtcttcttaattttctatatggattgaccacagtcttatcttccaccattaagctgtataacaataaaatgttaatagtcttgctttttattatcttttaaagatctccttaaattctataactgatcttttttcttattttgtttgtgacattcatacatttttaagatttttgttatgttctgaattcccccctacacacacacacacacacacacacacacacacgtgcaaaaaatatgatcaagaatgcaattgggatttgtgagcaatgagtagacctcttattgtttatatttgtaccctcattgtcaatttttttttagggaatttgggactctgcctatataaggtgttttaaatgtcttgagaacaagcactggctgatacctcttggagatatgatctgaaatgtaatggaatttattaaatggtgtttagtaaagtaggggttaaggacttgttaaagaaccccactatctctgagaccctatagccaaagcatgaggacttggagagctactaaaatgattcaggtttacaaaatgagccctgtgaggaaaggttgagagaagtctgaggagtttgtatttaattatagtcttccagtactgtatattcattcattactcattctacaaatatttattgaccccttttgatgtgcaaggcactatcgtgcgtcccctgagagttgcaagtatgaagcagtcatggatcatgaaccaaaggaacttatatgtagaggaaggataaatcacaaatagtgaatactgttagatacagatgatatattttaaaagttcaaaggaagaaaagaatgtgttaaacactgcatgagaggaggaataagtggcatagagctaggctttagaaaagaaaaatattccgataccatatgattggtgaggtaagtgttattctgagatgagaattagcagaaatagatatatcaatcggagtgattagagtgcagggtttctggaaagcaaggtttggacagagtggtcatcaaaggccagccctgtgacttacactgcattaaattaatttcttagaacatagtccctgatcattatcactttactattccaaaggtgagagaacagattcagatagagtgccagcattgtttcccagtattcctttacaaatcttgggttcattccaggtaaactgaactactgcattgtttctatcttaaaatactttttagatatcctagatgcatctttcaacttctaacattctgtagtttaggagttctcaaccttggcattattgacatgttaggccaaataattttttttgtgggaggtctcttgtgcgttttagatgattagcaataatccctgacctgttatctactaaagactagtcgtttctcatcagttgtgacaacaaaaatggttccagatattgccaaatgccctttagaggacagtaatcgcccccagttgagaaccatttcagtaaaactttaattactattttttcttttggtttataaaataatgatcctgaattaaattgatggaaccttgaagtcgataaaatatatttcttgctttaaagtccccatacgtgtcctactaattttctcatgctttagtgttttcacttttctcctgttatccttgtacctaagaatgccatcccaatccccagatgtccacctgcccaaagtctaggcatagctgaaggccaagctaaaatgtatccctctttttctggtacatgcagcaaaagtaatatgaattatcagctttctgagagcaggcattgtatctgtcttgtttggtgttacattggcacccaataaatatttgttgagtgaatgaataaattcccatagcactttattcttcacatggtacataactataggggctatagcttggtaccttgtgaagcaactcttggtgtaacataccttatttctcatactaaaatgcaagaacctttagagcaaggatcttgccattcatctttgtaacctctttactctggagcacttgcatttagcaggcatcataaagttttacgtaccaagaaaatgttgctgttttctgaatactatgcatcaaaaaatgttaccactaatttttaaagctctgctaaggaatattggggcaccctcagatgcaccttttaattgatgtcatattttcctaatccatactttattcatgagaatttgagtcaccccagcattagcttggaatttccttatttcccatttgctttgcaggtgccttggagtcagatctggttttgaatactatcttcctgttatgtgatcttgggcagttacttaattttctagtcaataacccgtatctataaaatagagaaaataatcctacacaccggggcctgttgtggggcggggagaggggggagggatcgcatttggagatatactaatgtaaatgacaagttaattggtgcagcacaccaacatggctcatgtctacatatgtaacaaacctgcacgttgtgcacatgtgccctagaacttaaagtataataaaaagaaattttaaaaaatcctgtcaaataaggttatagtagagaataaggatgtgtaaagcatttagtcacgtaaatgcttaaaaaaatgtaatttttacttctttcactgcctcatttaattagttttatctttaataataccttggattcagggtaaagtttcagttatgtcccagtaatcatttattttaccctcgaatctgcaatttggatagaacatggtggggacagctcgtctctattccttgcagcattaacaggctggaggcaccacttctctggccagcaagttgggcctggttgttggctgagagcctcagttcctttctgcacaggttcctctttacataggcttctcaacagggctactagagcatcgtcaccatagcagctgtcttataacagagagtggtcggtctgagagacaaaaaatggaagctgccaaattgttctgggtctggaaactgtcagggcatcacttgtgccatattcagttggcctaagaattacagagcctgcctcgattcaaagggagaggatagagaggactgaaggaatcagtgctcatctttaatatgcagcaggacaggtttgggattttttttcccccttgagtctgtgaaggcattacttaagaacaaagtcaggcatgtataattgaactacagttacttgaaatataagcccagaaagtttcagataataaatacaactatttttctgctgttacccttgtacctaaagatgccatcctaatccccagatctccacaactatacctacatagtagaaggttaaaatgtatccctctttttctggtgcatccagcaaaagtaatatcatgaattatgagctctctgagagcaaggatcatatcagtcttgtttattgttgcagtgaacaagtacagttgcagatattcaggagtaattatctaaatggcagtaggcttataaaactgaattttcaccagccacaccctccccccaactccttatctgtaaaaagcttatttgagtggttacctgtcttcagtaaagattgcgcttgcatatttgctgtcattgcatattctgcttaattaagctctgttgatattgcagtttctgtgcatacttacatcttagatgcaatctgagggcctaggaaggccttttaaaaataaaacaattccgattgcagagaaagtgtaagtcaaggacagttaattcaaggggaacatagaaagctatttagattttagttgatggtgccagtcttcagcgtaaagtcaaaagtggagggaagtttagtaaggaaaaaatgttgggcttggaatacattgtttagtcttcaaagcactttactttttatgaaatatattttagacattcagcaaatattgaatacttactatatcaggcagtaaagatataaattcattcttaaaatgtgcaacatgttcaaactgaaaaaaatacattcttaaacaggaaactttttccttcatactttttaattaacaagacatataagagttgcattaatgggcgtgcttatgattgatcacccagcagcatcattagaaataatatattttattcatgtgcagaaatcttttggttgtcctggggaaccttgaacacagaaaagagcttttattgataaggtaattgaacacacttgacaattagcttaatatggtttaataccatttgtgggagaagatgaatcagccaggctctttacgtcaagaatatgaagtttctcttgagtcaaccaacttaagatgagctacggagactgcagtgaaaagttaaatatccaagtacaccagccaatttcacacagtggaaccatgctgtcctcgggcaccctgcacctcgcccaacagtcatcaactagatggaggctcctggctgcaaggaggatttgatgggaatgagtaaatgtgtcagcatagtccgtcccttctaatggaaaagcaacccaaagagcaaatcctattaatggctggatcagtatcatctacttgtcaaaaacattccatgaattatgagtcaaaattttatttatggtggcattacacacattaagagatgaggacttctgttagcataatttattagctggaaaagttgagaaggttctctggactcatttttataggtggaacctaagtgatctggataattgcccaccagcaaaattgctgggcatggtggacaaagaaaatgttccttctaatgattttttatgagctgagtagctattgttcccagctgagtgctcttttcctctttttattgttgctgagcaaaagaatttataaaaagctctttcttttgtattaaaaaccctgctcaattgaaatgcaagttcattaagtaatcttcatttctcttcctgccataataaccctttccctctctgttcgattcaacagtatctagcagcactgctccaaattttaagtctgaacagactatattacatagatgtagagaaatactcaatcttcagcattaagagggagcttaatttcacacgggtggaatatgatcactcaggctagatgttggccataaatttcaaattagtatctcaacttagcaggggggatcaacagtggcaaacttcaattatgacaggataaaaatcacatagagatattggttcaatatggacatctaaactataatgctaaaagccaataattagaataagttcattttaagaaaagcattaataatattagctaacgtttagtacctgtgccaaacattctacctatgttaccttgattttcatagccagcctaagaggtactattatgtatccccattttacaggttaagaaacaggctcagaggagtttaggatcttttccaagattacatagccagtaagtggtggcactaggaaccaaattcagactctgaatcgcatgctgtttatattatattgcactcattctaaatatgtgggaatcagaatgaaggggcttgtatgacttttggctcattttttgatgcatgtgacctgggattataaatgtgaaattaggtttacgaaaggatccagtgtcattgtgcatcatgggcaaggagtacctaatctctttaattcttccctggaagcttacgatgtccatccaagtgcacatagcaaaagttctgttgtaaagtttagcagagtgactttctttgactcagagtgatgacggaggaagctttgataagattttatctgaaatgttcatggacaagagctttcaaggagaacatccagagcaaggttctgaagacagctcatgaaggtgaagcagcagacctggcacaagaaatgaagagagagctcagtgtattaaagatgaaaacaagaaaaccgaatatattgaaaggagcagagaggcaatgaaaacaagacaactgaaatgaggtaacttgcagcaattgaaagggaatttcagtacttttatagaattcttaaaaattgtttcctgctgtttattttcaattttgaacagggttatttgtccatgccatactttttttgccaaattccaaaattgtgtatagttctatagttgtctggtggagtcaatggaactttagttaccagtctaagaatgtgtctttgagattgtccagttaattctctatttccagtagctgtaataaatggtgaaaaggtttctgactcctggagaaagtttctaactccttatgactaatattcataacagacttgtgagttccttgaacatggatacacctatatgcaagagtgtattccaaagctaactcagtgatctttccatttatctattcttggattagtggtgcctttgctctttccttctgtaaatgtgaatagttaagagttgactgcagaagtgtttacactttggcttccatgcctctggaatgtttgtgctttggtggtgagatgtgagactatatttgtatagtctgcatctctcaggctgccccagaatgttgtacagtgcagtgctgaagaaagcagcaggtacacacagaaatgcagcctttcctggttaaccctgcttggatctgagttacactttgtttcctgacttcttgggacttaggtaatcagtttgccttctactctatctcattttgtactcgcttacatactacattcttgtttgggctttcgtttcttcttgtaagcagagattttttaaaatccaatatgtgaaaatacggatgcactacaattaaataaataaaatgctgttgtgtttgttttgctttaaaattgtaaaggataaacaataagatagttttatctatgtggttttcccgatgcagttaaaataaaacctaatctgctaaaattgaa

SEQ ID NO: 14 - DNA, 2013 bp, Homo sapiens
  source 1..2013: mol_type = other DNA
gcgaaggacatttgggctgtgtgtgcgacgcgggtcggaggggcagtcgggggaaccgcgaagaagccgaggagcccggagccccgcgtgacgctcctctctcagtccaaaagcggcttttggttcggcgcagagagacccgggggtctagcttttcctcgaaaagcgccgccctgcccttggccccgagaacagacaaagagcaccgcagggccgatcacgctgggggcgctgaggccggccatggtcatggaagtgggcaccctggacgctggaggcctgcgggcgctgctgggggagcgagcggcgcaatgcctgctgctggactgccgctccttcttcgctttcaacgccggccacatcgccggctctgtcaacgtgcgcttcagcaccatcgtgcggcgccgggccaagggcgccatgggcctggagcacatcgtgcccaacgccgagctccgcggccgcctgctggccggcgcctaccacgccgtggtgttgctggacgagcgcagcgccgccctggacggcgccaagcgcgacggcaccctggccctggcggccggcgcgctctgccgcgaggcgcgcgccgcgcaagtcttcttcctcaaaggaggatacgaagcgttttcggcttcctgcccggagctgtgcagcaaacagtcgacccccatggggctcagccttcccctgagtactagcgtccctgacagcgcggaatctgggtgcagttcctgcagtaccccactctacgatcagggtggcccggtggaaatcctgccctttctgtacctgggcagtgcgtatcacgcttcccgcaaggacatgctggatgccttgggcatcactgccttgatcaacgtctcagccaattgtcccaaccattttgagggtcactaccagtacaagagcatccctgtggaggacaaccacaaggcagacatcagctcctggttcaacgaggccattgacttcatagactccatcaagaatgctggaggaagggtgtttgtccactgccaggcaggcatttcccggtcagccaccatctgccttgcttaccttatgaggactaatcgagtcaagctggacgaggcctttgagtttgtgaagcagaggcgaagcatcatctctcccaacttcagcttcatgggccagctgctgcagtttgagtcccaggtgctggctccgcactgttcggcagaggctgggagccccgccatggctgtgctcgaccgaggcacctccaccaccaccgtgttcaacttccccgtctccatccctgtccactccacgaacagtgcgctgagctaccttcagagccccattacgacctctcccagctgctgaaaggccacgggaggtgaggctcttcacatcccattgggactccatgctccttgagaggagaaatgcaataactctgggaggggctcgagagggctggtccttatttatttaacttcacccgagttcctctgggtttctaagcagttatggtgatgacttagcgtcaagacatttgctgaactcagcacattcgggaccaatatatagtgggtacatcaagtccatctgacaaaatggggcagaagagaaaggactcagtgtgtgatccggtttctttttgctcgcccctgttttttgtagaatctcttcatgcttgacatacctaccagtattattcccgacgacacatatacatatgagaatataccttatttatttttgtgtaggtgtctgccttcacaaatgtcattgtctactcctagaagaaccaaatacctcaatttttgtttttgagtactgtactatcctgtaaatatatcttaagcaggtttgttttcagcactgatggaaaataccagtgttgggtttttttttagttgccaacagttgtatgtttgctgattatttatgacctgaaataatatatttcttcttctaagaagacattttgttacataaggatgacttttttatacaatggaataaattatggcatttctattgaaa

SEQ ID NO: 15 - DNA, 1253 bp, Homo sapiens
  source 1..1253: mol_type = other DNA
gagctatctgctcagcttaatagcaggacgctggcaacaggcactccctgctccagtccagcctgcgcgctccaccgccgctatggtctccgtgcctaccacctggtgctccgttgcgctagccctgctcgtggccctgcatgaagggaagggccaggctgctgccaccctggagcagccagcgtcctcatctcatgcccaaggcacccaccttcggcttcgccgttgctcctgcagctcctggctcgacaaggagtgcgtctacttctgccacttggacatcatctgggtgaacactcctgaacagacagctccttacggcctgggaaacccgccaagacgccggcgccgctccctgccaaggcgctgtcagtgctccagtgccagggaccccgcctgtgccaccttctgccttcgaaggccctggactgaagccggggcagtcccaagccggaagtcccctgcagacgtgttccagactggcaagacaggggccactacaggagagcttctccaaaggctgagggacatttccacagtcaagagcctctttgccaagcgacaacaggaggccatgcgggagcctcggtccacacattccaggtggaggaagagatagtgtcgtgagctggaggaacattgggaaggaagcccgcggggagagaggaggagagaagtggccagggcttgtggactctctgcctgcttcctggaccggggccttggtcccagacagctggacccatttgccaggattggcacaagctccctggtgagggagcctcgtccaaggcagttctgtgtcctcgcactgcccagggaagccctcggcctccagactgcggagcagcctccagtgctggctgctggcccacagctctgctggaagaactgcatggggagtacattcatctggaggctgcgtcctgaggagtgtcctgtctgctgggctacaaaccaggagcaaccgtgcagccacgaacacgcatgcctcagccagccctggaggctggatggctcccctgaggctggcatcctggctggctgtgtcctctccagctttccctccccagagttcttgcaccctcattccctcgggaccctcccagtgagaagggcctgctctgcttttcctgtctgtatataacttatttgccctaagaactttgagaatcccaattatttattttaatgtattttttagaccctctatttacctgcgaacttgtgtttataataaatgaggaaacata

SEQ ID NO: 16 - DNA, 5826 bp, Homo sapiens
  source 1..5826: mol_type = other DNA
agtatatgacagtacgtcagcagcgggatggccctagcagtggcggcggctgcagaagcccaagcagccgcggccgcagtggaggctagagccggagcggcggcggcggcggcaccccggggagtttaagatggcggcgggggggacagggggcctgcgggaggagcagcgctatgggctgtcgtgcggacggctggggcaggacaacatcaccgtactgcatgtgaagctcaccgagacggcgatccgggcgctcgagacttaccagagccacaagaatttaattccttttcgaccttcaatccagttccaaggactccacgggcttgtcaaaattcccaaaaatgatcccctcaatgaagttcataactttaacttttatttgtcaaatgtgggcaaagacaaccctcagggcagctttgactgcatccagcaaacattctccagctctggagcctcccagctcaattgcctgggatttatacaagataaaattacagtgtgtgcaacaaacgactcgtatcagatgacacgagaaagaatgacccaggcagaggaggaatcccgcaaccgaagcacaaaagttatcaaacccggtggaccatatgtagggaaaagagtgcaaattcggaaagcacctcaagctgtttcagatacagttcctgagaggaaaaggtcaacccccatgaaccctgcaaatacaattcgaaagacacatagcagcagcaccatctctcagaggccatacagggacagggtgattcacttactggccctgaaggcctacaagaaaccggagctacttgctagactccagaaagatggtgtcaatcaaaaagacaagaactccctgggagcaattctgcaacaggtagccaatctgaattctaaggacctctcatataccttaaaggattatgtttttaaagagcttcaaagagactggcctggatacagtgaaatagacagacggtcattggagtcagtgctctctagaaaactaaatccgtctcagaatgctgcaggcaccagccgttcagaatctcctgtatgttctagtagagacgctgtatcttctcctcagaaacggcttttggattcagagtttattgatcctttaatgaataaaaaagcccgaatatctcacctgacgaacagagtaccaccaacactaaatggtcatttgaatcccaccagtgaaaaatctgctgcaggcctcccgctgccccctgcggctgctgccatccctacccctccaccgctgccttcaacctatctgcccatctcacatcctcctcagattgtaaattctaactccaactcccctagcactccagaaggccgggggactcaagacctacctgttgacagttttagtcaaaacgatagtatctatgaggaccagcaagacaaatatacctctaggacttctctggaaaccttaccccctggttccgttctactaaagtgtccaaagcctatggaagaaaaccattcaatgtctcacaaaaagtccaaaaagaagtctaaaaaacataaggaaaaggaccaaataaaaaagcacgacattgagactattgaggaaaaggaggaagatcttaagagagaagaggaaattgccaagctaaataactccagtccaaattccagtggaggagttaaagaggattgcactgcctccatggaaccttcagcaattgaactcccagattatttgataaaatatatcgctatcgtctcctatgagcaacgccagaattataaggatgacttcaatgcagagtatgatgagtacagagctttgcatgccaggatggagactgtagctagaagatttatcaaactagatgcacaaagaaagcgcctttctccaggctcaaaagagtatcagaatgttcatgaagaagtcttacaagaatatcagaagataaagcagtctagtcccaattaccatgaagaaaaatacagatgtgaatatcttcataacaagctggctcacatcaaaaggctaataggtgaatttgaccaacagcaagcagagtcatggtcctagaactctgcttggaccagaagatgtgaataaacttaagcttatttatttaaaattccaaatgagttgctctagattctaaaaaggtgaaactttggctgttgaaagtttcagtattagtaaacttgagttactttttcttttccattttactttgcttccctgcatttcgaagctgctctttctggtcctccccaccaccccacccccaagacttgtgtttgttaatagaaataatttttttaggtattggggatccattgtctatatttcaaatcagttttttttcctcaaaaacttgtgtttgttattagaaatgattttttagatattggggatccagtgtccacacttaaaagttgtatgtgtttaaaaaacaacaacagtaatgtgcaaggtgaaatgcttttggataaacgtaagcctattttctgacgtttcttaatgcaaactctttgccttaaatggtagaatatttagaaatttgcacaaaattaaaaaaataaacattgtcttggagggttaaaaaatagaaaggtgtatgtgtatagattcacatacacatatgtatatacaggctgacttgatctagaacattaaatccgccctgcaagttaaccccccattgcaatggttgccttaaggtgtttgctagttgtgtacatagtgtggttaatcattagctacactgcttcccacttgattagagcaatgggaagcatactgtggcctaccagcatctggaagtgtgtgctcgatctgtatgtgtgcagaggtggtgtggatgtgagcgtgcatgaaggaaaaaaagctgctactcctagtaggccaaacgctcaggttaaacaactgacgagtgttactgtagggtgtttttttgtttttttgttttttttcttctatcaaattgctacttttgttgtggaagacaaaagcatttccatttcaacgagtttgtcagctttattaatgttgggcaaaaattgatatgtcatgaaaatgaaacagatctatagttttgggacaaaattataaaatgaaatgtgtaggtaacctatttatatactgctataaagtattttttgaagagagatatgcaaagaagctattacctacataagaggtatatttaaagattttttttttcatcctggtgccaggaatataaaaaagagtggatatatttaaccataacatactgtgattcatcaaacagcacaaactttcatttcatggagtttatctgttgacattgatttaaactgtcacttgttttatcatgtgggaacataagttatgtggtcaaaaatataaggattttgaattaatgttgattcaagttgtattgtcttattgtattgtcttttcaaagtgctgccagttgaaaagggaagcattatgtttacaaatctgttttgaaatgtttgccaaaattttggtagtgtctttaataaagatgtttgtctccagcatccagaaaaataaatgaataactttgttgtgtatcactgtaaaccagaaaaatgttggttatctagaaaacttgagagagcatgtagattaacttttctctttggagttctaaaacattaactggaaagattagataatatactaaatgtatacagaagtatacagactatacaaagactgaaacaagtcccttttgcactacaactctataacattaccgcagaaattttggttctatgtagcatggacctcctaaggaattctgtttcttttagcattgagatccctggtgctctttttttacctcagaattggtacaatcattattaaacgttaatttatttcaaactttttaattgaaaaaaggaaagggaaacttaattggggataaattcaggcatcatattattatgatagagtctcctgagtggttcgtctataggtaatgaactcattggtgttatttcttggacatcttggccttttaatcaaagactgtgtgctgctatttgctatgagcaaggtttctcaaaagcaaaaggtgcttggaccatttggatcacctgagttagaatctctaggtatagggcccaagtatctgcattttcacaggtttcttgtaggtgactttctgcaagctaaagtatgagaaccattggcttggatgtagttctaaacttttaggtctgtaaatcttgaaatcttgaactgaaggtcaactattggcttttttttttttttaatgtccatcatgtcagcaggtgcaaatcacttttcccctttgcatgatctgaggcacctcctcagttgtttcactgccaactcttgtttcagaacctgtttacaaacaagccttccagttggtgaatggttagccattggagctcctaccctgtacatcagcacatcttctggtttacaagttgggtaacaatgaaagctggagatgctaaatggaaatccagcattgcatacccttagacctgatcacataccagtaaaagccttaatttagatgttagttgtatgtgttggacagatccttgcaaaagtgtgtctgtctattagttgtaaatttgaaaattataaatctctgaatctgctactatccaagtttcatcccttttgaagatgaggcatgagcctattaaaatatttataatcatttttcgtcccctactgcaagacttttagattcttacaaatgattactacaggaatagtggccacttaatgtcagttactccggtggaagaatttatctagttttttttcttttcttttttggaaggatggtgtgaaaaatagcaagattagagaatgagttgtatagttttttctatcacatttcatctaaaatgatttgaaggacttttgaagatttttaccaacatccttaaatcaactccaggttggatgaacaactgatttaaaacaaactaagagaacattaactagatgtgggctttttaaaatatataggtattgcatttcctaccttgttatttattccactttgaatactttagagggcttaactttcaactctttaaggtagtaatggatagttttatacttgttctcacaaaattgttatggtcagtttatatcattgctccatgcattgattataaaaattcagtattaattttttctgatcttataagctttataggagttttcttttctcttataaagtgtttcaccttatgtaaaacaaatgcctgcttgcatattggaagatgttgaaattagttttagacaaaagtggtccatcaattcagacactctgcttggatgccttacccttttcattagtgcattctttgcttctgaaacttggcagaaactcgttagccagtccactgcctttctgacaatgtgtggagtcacgtatgcttggtatatgcctttactacttttaaagttctacagtttattacttgcccaagtgttactaaatccttttcttatgtgtactggatggagaaaaaattatagccagcactttgagaggaaagttttcagaaacaatattaactggcactactaactgaaggccacaggagatgctatcaatgttatttgtaatctgaagattgaacaaggctgtgaggctcatttcaaactattttgaggtgttaaaatatatatatgctgtttctcagctgttccactcaaaccgtgttaggactctcaaaggtaaaatgtcacaggggcttttcagttgttacagagctcagcagctgtggttgcccctgttctacaccaatttcagttcaataaaaatgttaactttgcaa

SEQ ID NO: 17 - DNA, 2102 bp, Homo sapiens
  source 1..2102: mol_type = other DNA
ctccgcctccggctccccgcactctccgggtccacgcatcgtcctcccgcgcgcccgcccgcccatggccgggaaggtgcggtcactgctgccgccgctgctgctggccgccgcgggcctcgccggcctcctactgctgtgcgtccccacccgcgacgtccgggagccgcccgccctcaagtatggcatcgtcctggacgctggttcttcacacacgtccatgtttatctacaagtggccggcagacaaggagaacgacacaggcattgtgggccagcacagctcctgtgatgttccaggtgggggcatctccagctatgcagacaacccttctggggccagccagagtcttgttggatgcctcgaacaggcgcttcaggatgtgcccaaagagagacacgcgggcacacccctctacctgggagccacagcgggtatgcgcctgctcaacctgaccaatccagaggcctcgaccagtgtgctcatggcagtgactcacacactgacccagtacccctttgacttccggggtgcacgcatcctctcgggccaggaagagggggtgtttggctgggtgactgccaactacctgctggagaacttcatcaagtacggctgggtgggccggtggttccggccacggaaggggacactgggggccatggacctggggggtgcctctacccagatcacttttgagacaaccagtccagctgaggacagagccagcgaggtccagctgcatctctacggccagcactaccgagtctacacccacagcttcctctgctatggccgtgaccaggtcctccagaggctgctggccagcgccctccagacccacggcttccacccctgctggccgaggggcttttccacccaagtgctgctcggggatgtgtaccagtcaccatgcaccatggcccagcggccccagaacttcaacagcagtgccagggtcagcctgtcagggagcagtgacccccacctctgccgagatctggtttctgggctcttcagcttctcctcctgccccttctcccgatgctctttcaatggggtcttccagcccccagtggctgggaactttgtggccttctctgccttcttctacactgtggactttttgcggacttcgatggggctgcccgtggccaccctgcagcagctggaggcagccgcagtgaatgtctgcaaccagacctgggctcagctgcaagctcgggtgccagggcaacgggcccgcctggccgactactgcgccggggccatgttcgtgcagcagctgctgagtcgcggctacggcttcgacgagcgcgccttcggcggcgtgatcttccagaagaaggccgcggacactgcagtgggctgggcgctcggctacatgctgaacctgaccaacctgatccccgccgacccgccggggctgcgcaagggcacagacttcagctcctgggtcgtcctcctgctgctcttcgcctccgcgctcctggctgcgcttgtcctgctgctgcgtcaggtgcactccgccaagctgccaagcaccatttaggggccgacgggggcagctgccccatccctcccccaacccctgtatccccaccccgtactcccacccctcccacaacccctgtacctcccacccctgtatccccacccctccacccacccctctcccaacctctctccccgcccctgtatcctgcattcctccacccaccctctatcccccaccgctccaccccaccactgtcttctccatccttccaccccaccctcagcgtctctgcccctaaggcagcccaggaaataggaactgagactctggtacccacaggagcctgggtgggcaaagagcgctcaatccagctccttgaacccctccagcccgcttcagcctgggcatcactgcaggccccgtgctcctcctcctcctcctcagggctgggtctccagagagtggggccttggtcctgagaatcagcccttagaggctccttctgtgtagtctgggtctgtactggggagggtcacagcccacgggctggcagccagcccagcacctacttgtaaaaattttgtaataaaaagtttttcctaga

SEQ ID NO: 18 - DNA, 8376 bp, Homo sapiens
  source 1..8376: mol_type = other DNA
ggagcacaaagacagcgattgttacccaataagcagtagtcacacacttctaccaatcgccttaggagggctggttaccatggaatccaccatctgttcactccccaccggaatgtgattatccttgctgggctgggaagtggggaaagcaggggagcctgtgctgggtgagattatctgcaatggaccgggttaatgggaggcttgctttggctcgctggaagaacaaatgaagggagttttacttcaaccatttgtgacttctggccaagaggaataaattcagcttttataaggcagcagtacctgtccaaagatcgctttagttccaccttgctccacaagcagcacaaccacactggttggtgagaatgtatctgaagaagaggctcagggaataaatgggaacaggccagcaaaacactcagctgcaagtccaaagccacaagtgcctccaaagccattacacctgcagaattcaccttcgtccaatatacaccaaacccccaggcataaagctttacctagtgcaaaaccaaggatggaggaaattaaacctgcctctgcttcttgtgtctcaaaagaaaaacccagtaaggtatcagatctcatcagtcgctttgaaggaggcagctcattatcaaattatagtgatttgaagaaagagtctgctgtgaacctaaatgctcctagaaccccaggaaggcatggattgacaaccacacctcaacaaaaactcctctcccagcacttgccacagaggcagggaaatgatacagataagactcagggtgcacagacttgtgtggccaacggtgtaatggcagcacaaaaccagatggaatgtgaggaggagaaagctgccactcttagctcagatacttctattcaagcttctgaacccttgcttgatacgcacatagtgaatggagaaagagatgaaactgccacagctcctgcatcacccacaacagacagctgtgatggaaatgcttctgacagtagctacaggactccaggcataggcccagtgctccccctagaagaaagaggggcagaaacagaaaccaaggtacaagagagggaaaatggggaaagccctctggaactggagcagctggaccagcaccatgagatgaaggagactaatgagcaaaaacttcacaaaatagccaatgaacttttgcttactgaaagagcttatgtcaaccgacttgacctcttagatcaggtattttattgcaaactgttggaagaagcaaaccgaggctcgtttccagcagagatggtgaataaaatcttttctaatatttcatcaataaatgccttccatagtaaattcctcttgccagagctggagaaacgaatgcaagaatgggaaactactcctagaattggagacatccttcagaaattggcaccattccttaagatgtatggagaatatgtgaaaggatttgataatgcaatggaattggttaaaaacatgacagaacgtattccccagttcaaatcagtggttgaagaaattcagaaacagaaaatctgtgggagcttaactttgcagcatcacatgctagaacctgttcagcggattccccggtatgagatgctccttaaggactatctaaggaaattgcctcctgattccctggactggaatgatgctaaaaaatcacttgaaattatatctacagcagcaagccattctaatagtgcaataaggaaaatggagaacctaaagaaactcttagagatttatgaaatgttgggagaagaagaagacattgtaaacccttcaaatgaactaataaaagaaggacagatcctcaaactagctgctcggaacacttcagcacaagaacgctaccttttcttattcaacaacatgttgctgtactgtgtgcccaaattcagcttggtaggctctaaattcacagttcgaaccagggttggcattgatggaatgaaaattgtagagactcaaaatgaagaatatccacatactttccaggtgtctgggaaagagagaacactggaactgcaggccagttctgcgcaagacaaagaagaatggatcaaggcccttcaagaaaccatcgatgcttttcatcaaaggcatgaaaccttcagaaatgcaattgcaaaggataatgacattcactcagaggtttctactgctgagctagggaaaagagccccaagatggatccgagataatgaagtgacaatgtgtatgaaatgtaaagaacctttcaatgcactgacacgaaggaggcatcattgtcgagcatgtggatatgtggtttgttggaaatgctccgactacaaagctcaacttgaatatgatggtggtaaattgagcaaagtttgtaaagactgttatcaaatcataagtggattcacagacagtgaagaaaagaaaagaaaaggaattttagagattgaatcagcagaagtatctggaaacagtgtggtgtgcagctttcttcagtatatggagaagtcaaaaccttggcagaaagcttggtgtgtgatccccaagcaagaccctcttgtgctgtacatgtatggtgccccccaggacgtcagagcccaggccaccattccacttctgggctatgtggtggatgaaatgccaaggagcgcagacctgccacacagtttcaaactgacccagtctaagtccgtgcacagctttgctgcagacagtgaggaactgaagcagaagtggctgaaagtcatccttttagctgtcacaggtgagacaccaggtggtccaaatgagcatccagccaccttggatgatcatcctgaacctaagaaaaaatcagaatgctgaactcctccaggaccagccatggtgtggaggtctcaggacttacagctcaagacattcccagctcttcttacacatctgctagcactttatgttgaaaaatataggcccataaatgcatcttttgaggactattttcctatgtttatgtactcttagtgaaattagtgtgcagagtcattctaccgataaagttttgaaataatgtgaaaactggagcattttttgagctattccttgaatatgtgcttttttgtcttgaagaaatggtgtatcaattgattctgtcaccgtcaggttagaatgagcacttccatttaagaaatcctttcatgtcttcttctctttcacatgtaggacctggaacagtttgaaagatatacctccatgttgccaaaatagatccatggtgaaaaatacagggacagttgaggctattgtattaacttatttaatttagtttataaatctctagctgcataaatgatgtctgttcttttaaaacaaaagaaaaaggacaaattgttggtgttcagatttccgatttataaagaaaagataacttgtttttgtagaaatactcctaagaatgtcttaagtatatagcaattatgtatatatagtattaaatatatatattatacatgcttttaggttcacattcatctctaatttattttttaaatataaagcatggtttatcgtggaattgagcaatgttctaaactgaagaaatgttttggtgatttatgttttgctgattggcatttgagggtattgatatttttataataagcggtaatttatgtggcatgggatatatttgtgaattccaacagtacttttaaagtaccattttttgtttctgtgcctatttaaaacagtgcctctcttagaaggtgctattaatataagatgagggttcagttaatgcttcagtcttgattattctaagattaaagtgcattttggtcactgggacagtcaaagtcatacaggttattttaccgtttacaatcctataattattgcagtagttaacatcagcatgtacaaacattttcaccaaaacccaaactttatgagacacctgaccttttacagaactaggatatgtggaaaccacatttatcgtaatgtttttcttttccgtaagataatcgagcatggttaaataaggtctctatataatgaacacttaagataactgtatgggccttttcctttgtcggaatgaaacaaaatccacatgtgataacctatgtttggggtgggggctggtcacatgattccttgtcatgaaggctgctcttcactcttttgtacttgtgattaaaatctcacactaagtttactatttaacttaatcctttttctcaaattaaacctttttttatactgtcataccccaaacattgtcactttttaatgtaccagtctgctagaaatctacaagaatgatcccactatctttagtatgtaaggattacaccttagatatagaatagaatttgagactgccacacatttattcaggccttgttactttaagaaatatcttattttttccaccccaaaggagcaagtgaaccaattattgctgacataataggttcccttcctccataaaaggattatcagccatgatcattaacattaaaccaaatagatggagccaaaaagagataaaagtagcttcttaggccctattaaatcagggaaagactagaagaaaatttacattgcttctcttttttaatggccagcggggtatgtgtgtgatgtatactcagagtggttttttggaaaattaaactgtagctatagatgatgctttggttttcttaatctcaagaaaagagaagcaatggaaatacagaatgattaactggggaagaggacttttgtaagtgtgactagaacaatggtaatgtatgggccaatgggaagatttataagtcagattatttttgaaaactatttttaaaagcaagaagactacactttccctaccaaaacagaagtaacatggacacacttaagttttggctcaataaatatcaatgttatttaacttagatcattgaaagaatactcatgtaaagataatgtttattatcatgctcttaacagtcattatctttaattttttaaatgagaagcattaatactagaagagaatttctggttatccggtcaccatattcactttccaccccacattcttcagctaaacgcaaagagaagcagtgaaacagccttacccgcttctctcttattaaagaatcactgatgtttttactcaatgaaaggtaaagtaatacccttagccatttattaaacaattcaaccaagagacctcaaagtgtgattgatgataagaataatcaatgccttttcccttccaacatacttgagcagtcatgacaacctaaaaatatcattggttgctttcccattaaaaccaacgttccctgtgggtcttaattctttattttcactcatttggtgctttccaagtcatcttttctttaaagtgcccttcctccaaactttaaaaagtacttccttgacaaaatttctattcattataaaacatttcaatattttaagcttaaattttgcttttgctgaaagcctaccatttggcgtgttaagtatgaaatatagtgcagacttttatcttggttttaagtggggctcaataaaaaacaccagccacttttgtaataatggcatcacagtgtcatcatgtatgcaagcaataaaactctttagggtatggttttatactgaaaatttaatatgaaggccccttcccacaagaatatagataattattaactttctagatgtgatacggtaattcgaattgcagagtataaggaagggaaatgggaaggggcatcattccttggattttaaataactatcaagaacatttttactttaacaaagaaaatggatagaaaaagcacattttgttttccctgaagtttaatttgacatcaggtttgtgtacttatcttcactaggtgacttaacttaccccaatttttttaaaaattattaaactttttacagaaactaaccttttaaatgtaccctttccccatatatatatacgcacacgtttggattttttttttttaagaacacttgttctagttataaatatataaagaaaacgataaagtttgtggtactgcagggttgttaaagattctttgatgccttctaaaaacttttgtcaaaaatacttttgagttcacaattctgttttacttttccttgtccttactttttggaaacagggtggttgcttttatttgttttctggttatattcaaagccttaagttcttaatctgagcatattgtctgtgataaatttctgatgatctttctggactagatacaacctgagtagcaagcaccaaccggagcaagtaaacttctagggaacaagcgtcttgggttttataggtatctttgctataatgcagaatagattaatgaagatttcctatatcatatgatatttgtgttagtgggtctaagattaagcacatgatatttataagctaaaattaactcaaaagtcaagaatgtcttaatgttttcattcttaaattttgtattctccaagaatgtattagtatatgaactgtggccaaccagttcctattcttcagactgtattgacatctgtagtggatcatgttgcttcttcattcttaccaattttattagaatcaaacttcttgttatttgcatactattatctactatagattctcagctttagaaaatgactatgatacataaagaccactaggtcaacttaaaaaaccctttctgtgaatttatacatgtatgtatatatgtaaaaacactgttgatttgcaattctgtctttccatagaaatgaactttttctatcgaaattgtttaacttaaatattttaacataaattatttacatggatctttatgtataattcatccttatatataccccttaatcacgtagtcatgagaagataactttgctttctttacagaaagggcagagaggaataagataagaaactgaaacaagcaagaatgaagagagatgtgggggagagactgctggtctccagaccacagcaatgtgttttaagataagatgaaatattttaactgcagaagggatataaaatctatgtaattacatgctgatgggatccattgcacccaggtttttgaccttggcctgtaaatgctagactatgatatcttgttattctttttctcctttgggcttttaaaaaataatttcattctcagatcattttctgtactgtttactgaggcaaaaaaaaaaaaaatctgaagtcaatcatggtcttctactttctggactgagcatttggcagaatgcagtatcttttcctgtattttgacatgaaatagcacatggcttctacaagatagttttaacttgttggggtcaccgggagttatatgatggtcaaccccttttcccaaaattcattgtggtagttttagtggaaaacgtaaatcaagaaatctcatatcatactttaataaataaataccaaatacatagtgacatataggtttgggaagaaactagtctgtggggaccattataagagaatcacattatatattacacagtatatggatattggaatgtatcacttgtggggggttctcttcattagcaaaacagtcatgtctgtctgtatataagacttttttttttttaaccaaactagcatttcattttgtgagtgacaattgacattttaaaataagcataggccgggcatagtggctcatgcctgtaatcccagcccttgggaggccgaggtgggcagatcacttcaggtcaggagtttgagaccagcctggccaacatggtgaaaccctgtctctactaaataaaaataaaacaaaataagtgtatctgggccaggcacggtagcccatgcctgtaatcccagcactttgggaggccgaggagttcgagaccagcctggccaacatggcaaaaccctgtctctacaaaaattagccaggtgtggtggcacatgcctgtaatcccagctactcgggaggttgaggcaggagaattgcttgaacccgggagacagaggttgcagtgagccgagattgcaccactgcactccggcctgggtgacagagcaagacttcgactcaaaaaataaaataagcgtatctggatttttcttttgaattcttgactgataagtttgagaatgtatgtttttggaattttacagttgactcaagtgtgaaagtatacacaagtaatttttcttttctagctatgtgaatgtaaaatacttgcagataatgtatatattgtgtaatatatgtatatttggtcataaaagcagataattggaaacattgtaaaattaagcactttaattcctattaaatattaaacatttgtatcttgtaaataaacacatccttaaattaaaaaaaaaaaaaaaaaaa

SEQ ID NO: 19 - DNA, 2291 bp, Homo sapiens
  source 1..2291: mol_type = other DNA
gaacaatgaagaaagccccacagccactgttgctgagcagggagaggatattacctccaaaaaagacaggggagtattaaagattgtcaaaagagtggggaatggtgaggaaacgccgatgattggagacaaagtttatgtccattacaaaggaaaattgtcaaatggaaagaagtttgattccagtcatgatagaaatgaaccatttgtctttagtcttggcaaaggccaagtcatcaaggcatgggacattggggtggctaccatgaagaaaggagagatatgccatttactgtgcaaaccagaatatgcatatggctcggctggcagtctccctaaaattccctcgaatgcaactctcttttttgagattgagctccttgatttcaaaggagaggatttatttgaagatggaggcattatccggagaaccaaacggaaaggagagggatattcaaatccaaacgaaggagcaacagtagaaatccacctggaaggccgctgtggtggaaggatgtttgactgcagagatgtggcattcactgtgggcgaaggagaagaccacgacattccaattggaattgacaaagctctggagaaaatgcagcgggaagaacaatgtattttatatcttggaccaagatatggttttggagaggcagggaagcctaaatttggcattgaacctaatgctgagcttatatatgaagttacacttaagagcttcgaaaaggccaaagaatcctgggagatggataccaaagaaaaattggagcaggctgccattgtcaaagagaagggaaccgtatacttcaagggaggcaaatacatgcaggcggtgattcagtatgggaagatagtgtcctggttagagatggaatatggtttatcagaaaaggaatcgaaagcttctgaatcatttctccttgctgcctttctgaacctggccatgtgctacctgaagcttagagaatacaccaaagctgttgaatgctgtgacaaggcccttggactggacagtgccaatgagaaaggcttgtataggaggggtgaagcccagctgctcatgaacgagtttgagtcagccaagggtgactttgagaaagtgctggaagtaaacccccagaataaggctgcaagactgcagatctccatgtgccagaaaaaggccaaggagcacaacgagcgggaccgcaggatatacgccaacatgttcaagaagtttgcagagcaggatgccaaggaagaggccaataaagcaatgggcaagaagacttcagaaggggtcactaatgaaaaaggaacagacagtcaagcaatggaagaagagaaacctgagggccacgtatgacgccacgccaaggagggaagagtcccagtgaactcggcccctcctcaatgggctttcccccaactcaggacagaacagtgtttaatgtaaagtttgttatagtctatgtgattctggaagcaaatggcaaaaccagtagcttcccaaaaacagcccccctgctgctgcccggagggttcactgaggggtggcacgggaccactccaggtggaacaaacagaaatgactgtggtgtggagggagtgagccagcagcttaagtccagctcatttcagtttctatcaaccttcaagtatccaattcagggtccctggagatcatcctaacaatgtggggctgttaggttttacctttgaactttcatagcactgcagaaaccttttaaaaaaaaatgcttcatgaatttctcctttcctacagttgggtagggtaggggaaggaggataagcttttgttttttaaatgactgaagtgctataaatgtagtctgttgcatttttaaccaacagaacccacagtagaggggtctcatgtctccccagttccacagcagtgtcacagacgtgaaagccagaacctcagaggccacttgcttgctgacttagcctcctcccaaagtccccctcctcagccagcctccttgtgagagtggctttctaccacacacagcctgtccctgggggagtaattctgtcattcctaaaacacccttcagcaatgataatgagcagatgagagtttctggattagcttttcctattttcgatgaagttctgagatactgaaatgtgaaaagagcaatcagaattgtgctttttctcccctcctctattccttttagggaataatattcaatacacagtacttcctcccagaaaaaaaaaaaaaaaaaaaaaaaaaaaaaaaaaaaaaaaaaaaaaaaaaaaaaaaaaaaaaaaaa

SEQ ID NO: 20 - DNA, 5437 bp, Homo sapiens
  source 1..5437: mol_type = other DNA
actgcagctgctgctgcctgctggttgccgcgtcttcccggaggcgactgctggaagctgcagggaacagctgcggtgctctgccatattgtgtcttccccggccggtccctcgcctcccggccgagctgacgttggttgaagccatggataacagaacccacagatgcagagggctgactgtacaagtctgtcaaaaaaccaaactgctggttggctaataattggtcactgtatgacttatagtacaggtgatattacttctgattctagatgaactgtaagagtaaatgggtccagtaattggaatgactccagataagagagctgaaaccccaggagctgaaaagattgcaggattaagccagatttacaaaatgggaagcttgcctgaagctgttgatgctgccaggccgaaggccactctagtggacagtgagtcagcagatgatgaactcacaaacttgaactggcttcatgaaagcactaatcttctaacaaacttcagcctcggaagtgagggtcttccaattgttagtccattgtatgacatagagggagatgatgtgccatcctttggaccagcttgctaccagaacccagaaaaaaaatcagcgacttcaaagcccccatactcctttagtcttctcatttatatggccattgagcactctccaaataaatgtttgcctgtcaaagaaatttatagctggattctggaccattttccatattttgctactgcaccaacaggctggaagaattctgttcgacataatctgtccctgaataaatgttttcagaaagtggaaagaagccatggcaaggttaatggaaaaggttccttatggtgtgttgatccggaatataaacccaatcttatccaggcactgaagaagcaacctttttcttcagcatcttcacaaaatggttctttatcacctcactatttaagctctgtaatcaagcagaaccaggtgcgaaacctcaaagaatctgatattgatgctgctgctgcaatgatgcttttaaatacttctatagaacaaggaattttagaatgtgagaagcctcttcctcttaaaacagcattgcaaaaaaagaggagttacggcaatgcatttcatcatcccagtgctgtacgattacaagagagtgattctttagccaccagcattgatccaaaagaagatcacaattacagtgcaagtagcatggcagcacagcgttgtgcatccaggtctagcgtgtcttccctgtcttctgtggatgaggtatatgaatttatcccaaagaatagtcacgtgggaagtgatggcagtgaaggatttcacagtgaagaagatacagacgttgattatgaagatgatcctcttggagacagtggctatgcatcacagccttgtgcaaaaatctctgaaaaagggcagtcaggcaaaaagatgcgaaaacagacatgtcaagaaattgatgaggagctcaaagaggcagctggatctctgctccaccttgctggaattcgtacatgtttaggttccctaataagtactgcaaagacacaaaatcaaaagcaacggaaaaaatagaaatacttaaagtgtggcaatactctttcacttaattctttacaagggatatcaaagccataatggacttcattagttttagggtagggaagggatactaattacttatttctttcaaaacatttttggtttttggtttttaaaatttttattaaacaattgctgttaggatcatgcctgatcagaaatacatgatgtgaagtcctaaaagcataatttttgtgataaatgtgtccaagatttgaaaatttttatataagaaaatccccagcctctgtcttaaacttgtgggacaaaaatcctttcttctgttaatatgtcagaatgtaagatttggcaactcgataaatttttttcattttcttttccaatctgtaactatagttactgaaccagctaaaaattttgctgatgtatttttcatatacttctctcctgacacgcaatccggtaacataagattgaaatttttttactttgtcttaatttgtttctaattttttttttgttgtcgtgtgagcaaagacaaacataaattgaaaaacatcctgatgtttagaaagtttcttttggttacgtaggtagagaatacaacaaagagttctaagacttagaaaagacaattttgtggtgtcatttcatccaagaacactcccaattcctattagaatggtagctttgaagtgtttttacttcagagatcctgcatagcatttattggggccttttattctttcccaaagtgctttgcaaacattaagttttagccactagctgcctttgttgagtataagttatccagtgagaggatatagccaattaagtgtgttatggagtacacccattttgacacacttgtaataagagaatctttcatttgcctgccatgtgtactgtattatgagaatcttataatcggaatggagtgaaatgaaacattttgagtactgatggcatgttatcatacataaagtaggtcatttaggaaaaaacttctgtccaagctttgtatgaattaaattcataagtataccaaattaaatcttaatagatttaatgcaattttacagacacaataccttcatgaatttcaaaattcatataaaatttgatcttatgcaatacacctttttgaggaggcagtgtaacaacctatagtgccattgatccatgagaaaaagattttctggtactactccaaaagtgctttgactaagtatcttaactatttgaacagtctgcttatatgagacagtttttcatagtatcatttgtataatttgattagattattcagaaacaataggatgctaaactaattcattgtatcttttttttacaatggtggaagcaactttctgaaagttcagtcttatgctacatcctaagtcatagacaatgaccttgttttcattattctgatagattgtatacatatgtacacatacatatacacatatgcacagtcaaggtcatgatgttgatttgctttcagctgtttctgtgattataaagcattttttaagagacaaattttaacttttaatttttattttggcaaaactgtcaaatgagaaaaatgattttaaaaacttttttttcaattgacaagactttaagtcagggataacagtattaatgttctctgttctgtttccatgttaaatttaatttaacctggatagccacattaatgaattggtgctgtcaaaatatagtaatttttaaatttgttaataatgaaaacccttaagcatgcaggatgaggtatttgggttttttttttagatcgatcacatctacagaaaatggctaaaccaagttaacttttattatagacagtgaataaaacaccaaaaacccaaaaatgctttaaccacagtataaataatagattatacacatcatcttaataactatttttaagttatttaccatagcctctgtatagaccttaggaacagtgtttcagtgatctggcaccagtttattttgttctgctgaaattctgtatcacaaatgtgctacctggtttttgtccattagataattactctttataaggaaaggaaagagaagcagagttagttccagctctaatagggatcttcaaagttattttgtcttgatgtatgtaacagtaattctttacatcttttgatttttctcttcctttttattcactcttcccacgaatttaaatgtttaagttatattcatcactagcaaggatgataaacacttgtgcactgaaagctaacagggagaggttacacaatattttacagtttcttaaacataatttaatgcatcaccttccacttgctaacataccaagtcagttattttcaagggaagaaccttttaaattatggtcctccatttactacttccactgagagtatgctctcattcctcaggtgttttgagaaacatgactaataaccacacaattaagtagagtcattccaagtcctatggcctggaaattgtattccctataatatacaaattttcctgtaataaagtcaacttagaaactccaaggaggttacatgttttccaacatatcctaaaaactgtgatataagctaacatataatttgccttacgtcaaaagaatatgttttgttgcagctgattccagtttataatagatccctagtaaaaagctttgattcaacacaattgttcatcttcacatcccaaacagaatcactgtttcttgaatatatatttttgaagtttttttgtgcaatatattactaaatcagttattattttacttttccaaattcagagaaagaaaacagattacctgaattcatggaaaaggtggatcacctccctttttccaccttcaagcctttcctgtcctcatagccagcatgacttcttttaacttggattcctttgtatatagtaaagtttagtatatatatatttttttctttttgctactttctgaggcattatgtaaagggctcatactagatgttcagttaaatatactttagcacaaagtcaaactagagaatgtgttaaggagggaatgtatatgtcttggtagaccaggaggcctttgccagcaatttaagcaacagatgtgaatacttcacaaagctgtaaagaccattgtcttaaatactacaacaacttaacacccttttgtgaagatcacagcatttatctaagaaactgtgaggctttctggtttacatatatcttacaggtgtttttttgtatttttttttttttttagtttgaaatgtgtaagctttgatttaaaccaagtttacttcagtatgttaatgatgtagtaaaaatatttattgaaaggtgaattcgagtattttaatgttatacctgccattttttttcttaaagcatattctttgcatctaactgccagtgccattgtcaaaacttattttttaaatcgttgtacatttcttattaaactaagtgcttaattttaaagtattatgttgccatcatatagtgtataaaaatgtataattgccaattgattgtaactattatttatttttaaatgaaagtgtaagaatgctttctgattcaacaaatttgttatcaaactgtttccttatcctcttttctgatgtagcataaaaattgtcccggtttgagttataactgccagtagatgaccagtcacaagtgaaccacttctcagttgccaatctttgctcatattaaaaacaacttacaaatacttagtttttgtatctaatctctgattattaaaatgtttataaagtttatttttaccaaagagatgcaattcattatgagaaagtattgcataataaattttgttttataactttt

SEQ ID NO: 21 - DNA, 1514 bp, Homo sapiens
  source 1..1514: mol_type = other DNA
atcggccaccgccatcccatccagcgagcatctgccgccgcgccgccgccaccctcccagagagcactggccaccgctccaccatcacttgcccagagtttgggccaccgcccgccgccaccagcccagagagcatcggcccctgtctgctgctcgcgcctggagatgtcagaggtccccgttgctcgcgtctggctggtactgctcctgctgactgtccaggtcggcgtgacagccggcgctccgtggcagtgcgcgccctgctccgccgagaagctcgcgctctgcccgccggtgtccgcctcgtgctcggaggtcacccggtccgccggctgcggctgttgcccgatgtgcgccctgcctctgggcgccgcgtgcggcgtggcgactgcacgctgcgcccggggactcagttgccgcgcgctgccgggggagcagcaacctctgcacgccctcacccgcggccaaggcgcctgcgtgcaggagtctgacgcctccgctccccatgctgcagaggcagggagccctgaaagcccagagagcacggagataactgaggaggagctcctggataatttccatctgatggccccttctgaagaggatcattccatcctttgggacgccatcagtacctatgatggctcgaaggctctccatgtcaccaacatcaaaaaatggaaggagccctgccgaatagaactctacagagtcgtagagagtttagccaaggcacaggagacatcaggagaagaaatttccaaattttacctgccaaactgcaacaagaatggattttatcacagcagacagtgtgagacatccatggatggagaggcgggactctgctggtgcgtctacccttggaatgggaagaggatccctgggtctccagagatcaggggagaccccaactgccagatatattttaatgtacaaaactgaaaccagatgaaataatgttctgtcacgtgaaatatttaagtatatagtatatttatactctagaacatgcacatttatatatatatgtatatgtatatatatatagtaactactttttatactccatacataacttgatatagaaagctgtttatttattcactgtaagtttattttttctacacagtaaaaacttgtactatgttaataacttgtcctatgtcaatttgtatatcatgaaacacttctcatcatattgtatgtaagtaattgcatttctgctcttccaaagctcctgcgtctgtttttaaagagcatggaaaaatactgcctagaaaatgcaaaatgaaataagagagagtagtttttcagctagtttgaaggaggacggttaacttgtatattccaccattcacatttgatgtacatgtgtagggaaagttaaaagtgttgattacataatcaaagctacctgtggtgatgttgccacctgttaaaatgtacactggatatgttgttaaacacgtgtctataatggaaacatttacaataaatattctgcatggaaa

SEQ ID NO: 22 - DNA, 3376 bp, Homo sapiens
  source 1..3376: mol_type = other DNA
gaagcccgaaccgaaggagcgggcatgaggcgctgcccgtgccgtgggagcctgaacgaggcggaggccggggcgctgcccgcggcggcccgcatgggactggaggcgccgcgaggagggcggcggcggcagccgggacagcagcgacctgggcccggcgcaggggccccggcggggcggccggaggggggcgggccctgggcccggacagaggggtccagcctccacagcgagcctgagagggccggcctcgggcctgcgccggggacagagagtccgcaggcagaattctggacagacggacagactgagcccgcggcagctggccttggagtagagaccgagaggcccaagcaaaagacggagccagacaggtccagcctccggacgcatctagaatggagctggtcagagctggagacgacttgtctttggacggagaccgggacagatggcctttggactgatccgcacaggtccgacctccagtttcagcccgaggaggccagcccctggacacagccaggggttcatgggccctggacagagctggaaacgcatgggtcacagactcagccagagagggtcaagtcctgggctgataacctctggacccaccagaacagttccagcctccagactcacccagaaggagcctgtccctcaaaagagccaagtgctgatggctcctggaaagaattgtatactgatggctccaggacacaacaggatattgaaggtccctggacagagccatatactgatggctcccagaaaaaacaggatactgaagcagccaggaaacagcctggcactggtggtttccaaatacaacaggatactgatggctcctggacacaacctagcactgacggttcccagacagcacctgggacagactgcctcttgggagagcctgaggatggcccattagaggaaccagagcctggagaattgctgactcacctgtactctcacctgaagtgtagccccctgtgccctgtgccccgcctcatcattacccctgagacccctgagcctgaggcccagccagtgggacccccctcccgggttgaggggggcagcggcggcttctcctctgcctcttctttcgacgagtctgaggatgacgtggtggccgggggcggaggtgccagcgatcccgaggacaggtctgggagcaaaccctggaagaagctgaagacagttctgaagtattcaccctttgtggtctccttccgaaaacactacccttgggtccagctttctggacatgctgggaacttccaggcaggagaggatggtcggattctgaaacgtttctgtcagtgtgagcagcgcagcctggagcagctgatgaaagacccgctgcgacctttcgtgcctgcctactatggcatggtgctgcaggatggccagaccttcaaccagatggaagacctcctggctgactttgagggcccctccattatggactgcaagatgggcagcaggacctatctggaagaggagctagtgaaggcacgggaacgtccccgtccccggaaggacatgtatgagaagatggtggctgtggaccctggggcccctacccctgaggagcatgcccagggtgcagtcaccaagccccgctacatgcagtggagggaaaccatgagctccacctctaccctgggcttccggatcgagggcatcaagaaggcagatgggacctgtaacaccaacttcaagaagacgcaggcactggagcaggtgacaaaagtgctggaggacttcgtggatggagaccacgtcatcctgcaaaagtacgtggcatgcctagaagaacttcgtgaagctctggagatctcccccttcttcaagacccacgaggtggtaggcagctccctcctcttcgtgcacgaccacaccggcctggccaaggtctggatgatagacttcggcaagacggtggccttgcccgaccaccagacgctcagccacaggctgccctgggctgagggcaaccgtgaggacggctacctctggggcctggacaacatgatctgcctcctgcaggggctggcacagagctgagctgctcagccaccatcaggttaattggatggcgccagtctggctggaggagccctgagatgccatgggaggcctgaggttggccacgggggagctggcctccagggacgggagagattgtgtcatgtgccacacgagaccaacgtggaaaagtctgaagggccttgggagaccaggtagcacctggccccatcatgatgcaggggttttggggacctggaaggaaggtgatgaggcagtgagtcagaaaaaccagaacggggtccccggatctgccgggaaggcttctgaggggctgccctgagagcattcagttcacatgtcacagggtatggtgtgacagggtgcctgtggacacatgaatcacttctaacctgcctccctgtcagcctccaggctgccagctggctgaggccagggactgggtcaggctcatctgtggcgcctcagagggtcagcatcattggtgaacagatgcaggcgctgctggaccatctggggagagtgacagtccatgtcttcaccagggagccatttgagtgctgagcgacaagaggctcagagggcatgaccccatgggactggatgcggcctgagggctgataccgctgggcggtatcctgccctgctgtggccctgtgggatcctccgtgttcctcggcggactctgctgacctcctgcagacccaaaccacagccacatcccagcttctgtgccagcactgtgacagtacctcgctcctctgtgcaccagatccggcctcaggacttacacctcctgcctgacccccaggcttctctctcctttctcccagcaaactgcagtggcagaaaggaggttcagaggctgggaaagtgggcctccccttgcaactcagagctgctgcactcaggagggccccatccaatcccgggcccctgcagggaaaagcgctgggtgtgtgtcagaggcgcagggtgggtggggctgccagccaggaccctggcctgcagcctgatccaaaccaaagactgtagaaccctggggtgtggctaacggcccctccagcacccatagccaggtcttcctggcccttgaggctgggctggcggacaggcacctacctcttccttaagctgaagctcccacactgtcttccagggctgaggagatgctctccttttctactgaccatcttgatacttatttatacgagaggcagttgctggacggggtagtactgggaagcaggaggcagaatggctctgctgagcctcctacccatgacaacaccccaataaacagaacattcagagccaa

SEQ ID NO: 23 - DNA, 2936 bp, Homo sapiens
  source 1..2936: mol_type = other DNA
ggcagtttcccgaccagagagaacgaacgtgtctgcgggcgcgcggggagcagaggcggtggcgggcggcggcggcaccgggagccgccgagtgaccctcccccgcccctctggccccccaccctcccacccgcccgtggcccgcgcccatggccgcgcgcgctccacacaactcaccggagtccgcgccttgcgccgccgaccagttcgcagctccgcgccacggcagccagtctcacctggcggcaccgcccgcccaccgccccggccacagcccctgcgcccacggcagcactcgaggcgaccgcgacagtggtgggggacgctgctgagtggaagagagcgcagcccggccaccggacctacttactcgccttgctgattgtctatttttgcgtttacaacttttctaagaacttttgtatacaaaggaactttttaaaaaagacgcttccaagttatatttaatccaaagaagaaggatctcggccaatttggggttttgggttttggcttcgtttcttctcttcgttgactttggggttcaggtgccccagctgcttcgggctgccgaggaccttctgggcccccacattaatgaggcagccacctggcgagtctgacatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaaatcccagacagtggatatgacccacactgccagaagagaattcagtattttttacttttcacactgtcttcccgatgagggaaggagcccagccagaaagcactacaatcatggtcaagttcccaactgagtcatcttgtgagtggataatcaggaaaaatgaggaatccaaaagacaaaaatcaaagaacagatggggtctgtgactggatcttctatcattccaattctaaatccgacttgaatattcctggacttacaaaatgccaagggggtgactggaagttgtggatatcagggtataaattatatccgtgagttgggggagggaagaccagaattcccttgaattgtgtattgatgcaatataagcataaaagatcaccttgtattctctttaccttctaaaagccattattatgatgttagaagaagaggaagaaattcaggtacagaaaacatgtttaaatagcctaaatgatggtgcttggtgagtcttggttctaaaggtaccaaacaaggaagccaaagttttcaaactgctgcatactttgacaaggaaaatctatatttgtcttccgatcaacatttatgacctaagtcaggtaatatacctggtttacttctttagcatttttatgcagacagtctgttatgcactgtggtttcagatgtgcaataatttgtacaatggtttattcccaagtatgccttaagcagaacaaatgtgtttttctatatagttccttgccttaataaatatgtaatataaatttaagcaaacgtctattttgtatatttgtaaactacaaagtaaaatgaacattttgtggagtttgtattttgcatactcaaggtgagaattaagttttaaataaacctataatattttatctgaa

SEQ ID NO: 24 - DNA, 3950 bp, Homo sapiens
  source 1..3950: mol_type = other DNA
acttctcacttccgcttccttccagtaaggagtcggggtcttccccagttttctcagccaggcggcggcggcgactggcaatgtttggcctcaaaagaaacgcggtaatcggactcaacctctactgtgggggggccggcttgggggccggcagcggcggcgccacccgcccgggagggcgacttttggctacggagaaggaggcctcggcccggcgagagatagggggaggggaggccggcgcggtgattggcggaagcgccggcgcaagccccccgtccaccctcacgccagactcccggagggtcgcgcggccgccgcccattggcgccgaggtccccgacgtcaccgcgacccccgcgaggctgcttttcttcgcgcccacccgccgcgcggcgccgcttgaggagatggaagccccggccgctgacgccatcatgtcgcccgaagaggagctggacgggtacgagccggagcctctcgggaagcggccggctgtcctgccgctgctggagttggtcggggaatctggtaataacaccagtacggacgggtcactaccctcgacgccgccgccagcagaggaggaggaggacgagttgtaccggcagtcgctggagattatctctcggtaccttcgggagcaggccaccggcgccaaggacacaaagccaatgggcaggtctggggccaccagcaggaaggcgctggagaccttacgacgggttggggatggcgtgcagcgcaaccacgagacggccttccaaggcatgcttcggaaactggacatcaaaaacgaagacgatgtgaaatcgttgtctcgagtgatgatccatgttttcagcgacggcgtaacaaactggggcaggattgtgactctcatttcttttggtgcctttgtggctaaacacttgaagaccataaaccaagaaagctgcatcgaaccattagcagaaagtatcacagacgttctcgtaaggacaaaacgggactggctagttaaacaaagaggctgggatgggtttgtggagttcttccatgtagaggacctagaaggtggcatcaggaatgtgctgctggcttttgcaggtgttgctggagtaggagctggtttggcatatctaataagatagccttactgtaagtgcaatagttgacttttaaccaaccaccaccaccaccaaaaccagtttatgcagttggactccaagctgtaacttcctagagttgcaccctagcaacctagccagaaaagcaagtggcaagaggattatggctaacaagaataaatacatgggaagagtgctccccattgattgaagagtcactgtctgaaagaagcaaagttcagtttcagcaacaaacaaactttgtttgggaagctatggaggaggacttttagatttagtgaagatggtagggtggaaagacttaatttccttgttgagaacaggaaagtggccagtagccaggcaagtcatagaattgattacccgccgaattcattaatttactgtagtgttaagagaagcactaagaatgccagtgacctgtgtaaaagttacaagtaatagaactatgactgtaagcctcagtactgtacaagggaagcttttcctctctctaattagctttcccagtatacttcttagaaagtccaagtgttcaggacttttatacctgttatactttggcttggtttccatgattcttactttattagcctagtttatcaccaataatacttgacggaaggctcagtaattagttatgaatatggatatcctcaattcttaagacagcttgtaaatgtatttgtaaaaattgtatatatttttacagaaagtctatttctttgaaacgaaggaagtatcgaatttacattagtttttttcatacccttttgaactttgcaacttccgtaattaggaacctgtttcttacagcttttctatgctaaactttgttctgttcagttctagagtgtatacagaacgaattgatgtgtaactgtatgcagactggttgtagtggaacaaatctgataactatgcaggtttaaattttcttatctgattttggtaagtattccttaggtttttctttgaaaacctgggattgagaggttgatgaatggaaattctttcacttcattatatgcaagttttcaataattaggtctaagtggagttttaaggttactgatgacttacaaataatgggctctgattgggcaatactcatttgagttccttccatttgacctaatttaactggtgaaatttaaagtgaattcatgggctcatctttaaagcttttactaaaagattttcagctgaatggaactcattagctgtgtgcatataaaaagatcacatcaggtggatggagagacatttgatcccttgtttgcttaataaattataaaatgatggcttggaaaagcaggctagtctaaccatggtgctattattaggcttgcttgttacacacacaggtctaagcctagtatgtcaataaagcaaatacttactgttttgtttctattaatgattcccaaaccttgttgcaagtttttgcattggcatctttggatttcagtcttgatgtttgttctatcagacttaaccttttatttcctgtccttccttgaaattgctgattgttctgctccctctacagatatttatatcaattcctacagctttcccctgccatccctgaactctttctagcccttttagattttggcactgtgaaacccctgctggaaacctgagtgaccctccctccccaccaagagtccacagacctttcatctttcacgaacttgatcctgttagcaggtggtaataccatgggtgctgtgacactaacagtcattgagaggtgggaggaagtcccttttccttggactggtatcttttcaactattgttttatcctgtctttgggggcaatgtgtcaaaagtcccctcaggaattttcagaggaaagaacattttatgaggctttctctaaagtttcctttgtataggagtatgctcacttaaatttacagaaagaggtgagctgtgttaaacctcagagtttaaaagctactgataaactgaagaaagtgtctatattggaactagggtcatttgaaagcttcagtctcggaacatgacctttagtctgtggactccatttaaaaataggtatgaataagatgactaagaatgtaatggggaagaactgccctgcctgcccatctcagagccataaggtcatctttgctagagctatttttacctatgtatttatcgttcttgatcataagccgcttatttatatcatgtatctctaaggacctaaaagcactttatgtagtttttaattaatcttaagatctggttacggtaactaaaaaagcctgtctgccaaatccagtggaaacaagtgcatagatgtgaattggtttttaggggccccacttcccaattcattaggtatgactgtggaaatacagacaaggatcttagttgatattttgggcttggggcagtgagggcttaggacaccccaagtggtttgggaaaggaggaggggagtggtgggtttatagggggaggaggaggcaggtggtctaagtgctgactggctacgtagttcgggcaaatcctccaaaagggaaagggaggatttgcttagaaggatggcgctcccagtgactactttttgacttctgtttgtcttacgcttctctcagggaaaaacatgcagtcctctagtgtttcatgtacattctgtggggggtgaacaccttggttctggttaaacagctgtacttttgatagctgtgccaggaagggttaggaccaactacaaattaatgttggttgtcaaatgtagtgtgtttccctaactttctgtttttcctgagaaaaaaaaataaatcttttattcaaa

SEQ ID NO: 25 - DNA, 401 bp, Homo sapiens
  source 1..401: mol_type = other DNA
accacgcctcctccaagtcccagcgaacccgcgtgcaacctgtcccgactctagccgcctcttcagctcgccatggatcccaactgctcctgcgccgccggtgactcctgcacctgcgccggctcctgcaaatgcaaagagtgcaaatgcacctcctgcaagaaaagctgctgctcctgctgccctgtgggctgtgccaagtgtgcccagggctgcatctgcaaaggggcgtcggacaagtgcagctgctgcgcctgatgctgggacagccccgctcccagatgtaaagaacgcgacttccacaaacctggattttttatgtacaaccctgaccgtgaccgtttgctatattcctttttctatgaaataatgtgaatgataataaaacagctttgacttga

SEQ ID NO: 26 - DNA, 1559 bp, Homo sapiens
  source 1..1559: mol_type = other DNA
acagcagtccgtgccgccgtcccgcccgccagcgccccagcgaggaagcagcgcgcagcccgcggcccagcgcacccgcagcagcgcccgcagctcgtccgcgccatgttccaggcggccgagcgcccccaggagtgggccatggagggcccccgcgacgggctgaagaaggagcggctactggacgaccgccacgacagcggcctggactccatgaaagacgaggagtacgagcagatggtcaaggagctgcaggagatccgcctcgagccgcaggaggtgccgcgcggctcggagccctggaagcagcagctcaccgaggacggggactcgttcctgcacttggccatcatccatgaagaaaaggcactgaccatggaagtgatccgccaggtgaagggagacctggccttcctcaacttccagaacaacctgcagcagactccactccacttggctgtgatcaccaaccagccagaaattgctgaggcacttctgggagctggctgtgatcctgagctccgagactttcgaggaaatacccccctacaccttgcctgtgagcagggctgcctggccagcgtgggagtcctgactcagtcctgcaccaccccgcacctccactccatcctgaaggctaccaactacaatggccacacgtgtctacacttagcctctatccatggctacctgggcatcgtggagcttttggtgtccttgggtgctgatgtcaatgctcaggagccctgtaatggccggactgcccttcacctcgcagtggacctgcaaaatcctgacctggtgtcactcctgttgaagtgtggggctgatgtcaacagagttacctaccagggctattctccctaccagctcacctggggccgcccaagcacccggatacagcagcagctgggccagctgacactagaaaaccttcagatgctgccagagagtgaggatgaggagagctatgacacagagtcagagttcacggagttcacagaggacgagctgccctatgatgactgtgtgtttggaggccagcgtctgacgttatgagcgcaaaggggctgaaagaacatggacttgtatatttgtacaaaaaaaaagttttatttttctaaaaaaagaaaaaagaagaaaaaatttaaagggtgtacttatatccacactgcacactgcctggcccaaaacgtcttattgtggtaggatcagccctcattttgttgcttttgtgaactttttgtaggggacgagaaagatcattgaaattctgagaaaacttcttttaaacctcacctttgtggggtttttggagaaggttatcaaaaatttcatggaaggaccacattttatatttattgtgcttcgagtgactgaccccagtggtatcctgtgacatgtaacagccaggagtgttaagcgttcagtgatgtggggtgaaaagttactacctgtcaaggtttgtgttaccctcctgtaaatggtgtacataatgtattgttggtaattattttggtacttttatgatgtatatttattaaacagatttttacaaa

SEQ ID NO: 27 - DNA, 4788 bp, Homo sapiens
  source 1..4788: mol_type = other DNA
tttttagaaaaaaaaaatatatttccctcctgctccttctgcgttcacaagctaagttgtttatctcggctgcggcgggaactgcggacggtggcgggcgagcggctcctctgccagagttgatattcactgatggactccaaagaatcattaactcctggtagagaagaaaaccccagcagtgtgcttgctcaggagaggggagatgtgatggacttctataaaaccctaagaggaggagctactgtgaaggtttctgcgtcttcaccctcactggctgtcgcttctcaatcagactccaagcagcgaagacttttggttgattttccaaaaggctcagtaagcaatgcgcagcagccagatctgtccaaagcagtttcactctcaatgggactgtatatgggagagacagaaacaaaagtgatgggaaatgacctgggattcccacagcagggccaaatcagcctttcctcgggggaaacagacttaaagcttttggaagaaagcattgcaaacctcaataggtcgaccagtgttccagagaaccccaagagttcagcatccactgctgtgtctgctgcccccacagagaaggagtttccaaaaactcactctgatgtatcttcagaacagcaacatttgaagggccagactggcaccaacggtggcaatgtgaaattgtataccacagaccaaagcacctttgacattttgcaggatttggagttttcttctgggtccccaggtaaagagacgaatgagagtccttggagatcagacctgttgatagatgaaaactgtttgctttctcctctggcgggagaagacgattcattccttttggaaggaaactcgaatgaggactgcaagcctctcattttaccggacactaaacccaaaattaaggataatggagatctggttttgtcaagccccagtaatgtaacactgccccaagtgaaaacagaaaaagaagatttcatcgaactctgcacccctggggtaattaagcaagagaaactgggcacagtttactgtcaggcaagctttcctggagcaaatataattggtaataaaatgtctgccatttctgttcatggtgtgagtacctctggaggacagatgtaccactatgacatgaatacagcatccctttctcaacagcaggatcagaagcctatttttaatgtcattccaccaattcccgttggttccgaaaattggaataggtgccaaggatctggagatgacaacttgacttctctggggactctgaacttccctggtcgaacagttttttctaatggctattcaagccccagcatgagaccagatgtaagctctcctccatccagctcctcaacagcaacaacaggaccacctcccaaactctgcctggtgtgctctgatgaagcttcaggatgtcattatggagtcttaacttgtggaagctgtaaagttttcttcaaaagagcagtggaaggacagcacaattacctatgtgctggaaggaatgattgcatcatcgataaaattcgaagaaaaaactgcccagcatgccgctatcgaaaatgtcttcaggctggaatgaacctggaagctcgaaaaacaaagaaaaaaataaaaggaattcagcaggccactacaggagtctcacaagaaacctctgaaaatcctggtaacaaaacaatagttcctgcaacgttaccacaactcacccctaccctggtgtcactgttggaggttattgaacctgaagtgttatatgcaggatatgatagctctgttccagactcaacttggaggatcatgactacgctcaacatgttaggagggcggcaagtgattgcagcagtgaaatgggcaaaggcaataccaggtttcaggaacttacacctggatgaccaaatgaccctactgcagtactcctggatgtttcttatggcatttgctctggggtggagatcatatagacaatcaagtgcaaacctgctgtgttttgctcctgatctgattattaatgagcagagaatgactctaccctgcatgtacgaccaatgtaaacacatgctgtatgtttcctctgagttacacaggcttcaggtatcttatgaagagtatctctgtatgaaaaccttactgcttctctcttcagttcctaaggacggtctgaagagccaagagctatttgatgaaattagaatgacctacatcaaagagctaggaaaagccattgtcaagagggaaggaaactccagccagaactggcagcggttttatcaactgacaaaactcttggattctatgcatgaagtggttgaaaatctccttaactattgcttccaaacatttttggataagaccatgagtattgaattccccgagatgttagctgaaatcatcaccaatcagataccaaaatattcaaatggaaatatcaaaaaacttctgtttcatcaaaagtgactgccttaataagaatggttgccttaaagaaagtcgaattaatagcttttattgtataaactatcagtttgtcctgtagaggttttgttgttttattttttattgttttcatctgttgttttgttttaaatacgcactacatgtggtttatagagggccaagacttggcaacagaagcagttgagtcgtcatcacttttcagtgatgggagagtagatggtgaaatttattagttaatatatcccagaaattagaaaccttaatatgtggacgtaatctccacagtcaaagaaggatggcacctaaaccaccagtgcccaaagtctgtgtgatgaactttctcttcatactttttttcacagttggctggatgaaattttctagactttctgttggtgtatcccccccctgtatagttaggatagcatttttgatttatgcatggaaacctgaaaaaaagtttacaagtgtatatcagaaaagggaagttgtgccttttatagctattactgtctggttttaacaatttcctttatatttagtgaactacgcttgctcattttttcttacataattttttattcaagttattgtacagctgtttaagatgggcagctagttcgtagctttcccaaataaactctaaacattaatcaatcatctgtgtgaaaatgggttggtgcttctaacctgatggcacttagctatcagaagaccacaaaaattgactcaaatctccagtattcttgtcaaaaaaaaaaaaaaaaaagctcatattttgtatatatctgcttcagtggagaattatataggttgtgcaaattaacagtcctaactggtatagagcacctagtccagtgacctgctgggtaaactgtggatgatggttgcaaaagactaatttaaaaaataactaccaagaggccctgtctgtacctaacgccctatttttgcaatggctatatggcaagaaagctggtaaactatttgtctttcaggaccttttgaagtagtttgtataacttcttaaaagttgtgattccagataaccagctgtaacacagctgagagacttttaatcagacaaagtaattcctctcactaaactttacccaaaaactaaatctctaatatggcaaaaatggctagacacccattttcacattcccatctgtcaccaattggttaatctttcctgatggtacaggaaagctcagctactgatttttgtgatttagaactgtatgtcagacatccatgtttgtaaaactacacatccctaatgtgtgccatagagtttaacacaagtcctgtgaatttcttcactgttgaaaattattttaaacaaaatagaagctgtagtagccctttctgtgtgcaccttaccaactttctgtaaactcaaaacttaacatatttactaagccacaagaaatttgatttctattcaaggtggccaaattatttgtgtaatagaaaactgaaaatctaatattaaaaatatggaacttctaatatatttttatatttagttatagtttcagatatatatcatattggtattcactaatctgggaagggaagggctactgcagctttacatgcaatttattaaaatgattgtaaaatagcttgtatagtgtaaaataagaatgatttttagatgagattgttttatcatgacatgttatatattttttgtaggggtcaaagaaatgctgatggataacctatatgatttatagtttgtacatgcattcatacaggcagcgatggtctcagaaaccaaacagtttgctctaggggaagagggagatggagactggtcctgtgtgcagtgaaggttgctgaggctctgacccagtgagattacagaggaagttatcctctgcctcccattctgaccacccttctcattccaacagtgagtctgtcagcgcaggtttagtttactcaatctccccttgcactaaagtatgtaaagtatgtaaacaggagacaggaaggtggtgcttacatccttaaaggcaccatctaatagcgggttactttcacatacagccctcccccagcagttgaatgacaacagaagcttcagaagtttggcaatagtttgcatagaggtaccagcaatatgtaaatagtgcagaatctcataggttgccaataatacactaattcctttctatcctacaacaagagtttatttccaaataaaatgaggacatgtttttgttttctttgaatgctttttgaatgttatttgttattttcagtattttggagaaattatttaataaaaaaacaatcatttgctttttg

SEQ ID NO: 28 - DNA, 7040 bp, Homo sapiens
  source 1..7040: mol_type = other DNA
gcctcagggaagcgagcccgggcgccggcgggcggccgtcgcgtctgacagaccactgcagaccacgggccgaggcccagcgcccgtccgcagcgcggccggcatggcggcgacaaggagccccacgcgggcaagggagcgggagcggtctggcgctcccgccgcaggaagtgaccaagttcactcctggatgctagctacaagccaagccttagacactgtctggagaatggcaaaaggctttgtgatgttggcagtttcatttctggtggctgccatctgctacttccggaggctacatttatattcagggcacaagctgaaatggtggattggatatctgcagagaaaattcaaaaggaacctcagtgtggaggcagaggttgatttactcagttattgtgcaagagaatggaaaggagagacaccccgtaacaagctgatgaggaaggcttatgaggagctattttggcggcatcacattaaatgtgttcgacaagtaaggagagataactatgatgctctcagatcagtgttatttcagatattcagccagggcatctcttttccatcatggatgaaagaaaaggacattgttaagcttcctgaaaaactgctgttttcacaaggttgtaattggattcagcagtacagttttggtcctgagaagtatacaggctcgaatgtgtttggaaaactacggaaatatgtggaattattgaaaacacagtggactgaatttaatggcattagagattatcacaagagaggaagtatgtgcaacacccttttttcagatgccattctggaatataaactttatgaagctttaaagttcatcatgctgtatcaagtcactgaagtttatgaacaaatgaagactaaaaaggtcattcccagtctttttagactcctgttttccagggagacatcctctgatcctttgagcttcatgatgaatcacctgaattctgtaggcgacacatgtggactagagcagattgatatgtttatacttggatactcccttgaagtaaagataaaagtgttcagactgttcaagtttaactccagagactttgaagtctgctacccagaggagcctctcagggactggccggagatctccctgctgaccgagaacgaccgccactaccacattccagtcttttaagtccgctgggggccgaacagcagtgctcaccagtgacggtggtcacagttgcaataaagtctctctctgaaaccaaagctagcatttcagcatggaaggaattaggaccttttcttcaggattacaggtacactggatgcagccatgcatggatggtttttctttatttttcagtgatttcctctgaagcagctgcactgatacatttgggagttggtggcttgactttgtccataaggggcgtggccacttcacatgatggcgggcctttaagagcacaaagaagtttaatatggacaacaacaggaaaaagcaagaagaaaacaagtagggaaaaacagctaacctggagagaaagaatttctttaacctttatgttcttcattaaaaatcttatcttggactgatttgagggatttttagaaacatggccttattttatataagcattaccttcccaggaatctttgttgtatattaatttttgataaccatttgattaactttaaaattaagtatatgtgtgtatatatacatatgtatgtttatatacacacatgtatctgtatagttttatatatacatatatacacatagacatacagagaaccactactttgtaatagtgtacagtttgttttatatctctttactttttttgttactattttatctggccagcttaatagttttatttagattttttaaaattctgtagattaaagcaaatgacagttattgaactatcacaaaactattaaactgtggtacatttaatgtgtatttaatgtgtggaatctcattatggtaggcagaataatgccctatgctccccaaagatgtctgtgtctgaatcctcagagccgtaaagggctaaatgggttaaatgggaaaggagaattaaggttgcaggtggaactgagatgctaatccaatgatgctgaaatagggagatttttctagattctccaggtggacgcagtgtaatcagaatggttcttaaaagtggaagagggaattggaagagtgtcagagtcagaggggaatgtgaaatatggaagaacggttagagagagatgcaccattgctggctttgaagatggaggaaggggccataagccagggagttgcaggcagcctgtagaattgagactggaaaggaaacagaaacttccccatagcctccagaaggaacgcagccctgccaacaccttgattttagcccagtgaagcctgtgttggacctctaatctgcagaactgtaagataatagatttgtgtttaagccactaagtttgtggtaatgtgttgtagcaacagaaaactagtattctcaggctagtttttggtcgtctttattttggaaatacttttctagtttaatttgatagttttttttttggaggtactttgctagtctgttttgagtttcagtacataagaataatttttaaaatcttgctaattatatattacttcaaagtcacaaatccctttctaagaatgacttacttcacgagatatttagcactatcattttcccagatgttcatattatttctgcaataaattaaaagggagtgtgtcaaatgctgtcatgtctgaaattagcattcatattcttttgcaatggggatgatcagtcgtgtgtactacatgaaccatgtctgatggtagcttgttcccactgtcattttgttttctggttgaagattaatgagctcagccacagaaacaagagttcttcattgacctctacagtccctgcctgtttggaaacatctatggttttgtataacccctgtcatttaactgacagtgttagaagatatccccctgatgtgttactgtaaccaagaaagcatgaacgttaccctttctggtgacagcctgccatgggctgctgtggctgatacttatagaattgttgctccaaaattttggctccacttgagctgtccagaagtgtacctgacatttgtgtattaccactcaccttggatctccttactagtgtaattatttccacatcaatccaggactgaaaggaaaaattttttcaccaggattggcagcctgtagctctgtgacctcagtcaacccatcatgttgtgttgtgggcggggggcaagaattctttagagaaccaagttgcgagaaagatttcaatccagtgaaagtaaaaagtaagaagacatttagatagttgctatattttgggaagatgtcaaaacaggtttttagggaggaggtatgaggtgttgtgttgttttgtttgtttttattcagttgtataatgaatataacaaattaagtagccagaaggagctgcatgtaaattagcaccacttttaaatgtcaacaataaatttgaggtgagcttcctgggtgatgccaacatttaaatgtctttctaaccgtatatgttttaaatggtgagagaactatagcaaaaatggaaacataatgccctcgtcgttttttgattttaggataagtttctctctcaaattttggccttacgtgtccatactgaggggttgtatgcatattagtacaaggctgacttttactgtggtaaaatacacataacatatagtttatattttaacaatttttttttttgagacggagtttcactctttttgctcaggctgcagtgcagtggcacaatctcggctcactgcaacctctacctcccaggttcaagcgattctcctgcctcagcctcccgagtagccgggattacaggtgcccaccactacgctcgactaattttttgtgtttttagtagaaacggggtttcaccatttagccaggctgatctgaactcctgaccttgggcgatccgcctacctcggcatcccaaagtgctgggattacgggtgtgagccactgcgcccggccctaacaatttttaagtgacagtgacattaagcatattcacattgttgtgcaaccctcaccaccatccaccctcagaactttttaaaatctccaagactgactttttttcaaagcaggcaactttaattccctacctggtatctggattccttttccttttcatgctatcttttcatcatacctcttctaatctgagtatttcctctgggcttaaaagagcctcagtggagaagtacaacctaagagggagttaggtacaacctaggagggagtcaggaggagagagttaggttagttagtacaacctaagagggagttaaatggatcaggaaaatcgtgttttatctccaaagtaaaatgataactatgggtggcttctgggctcaatttagaatattatcattgaaaagtccccaagaaactattaattcagagccaccctggtgagttgaatttcttgaatgtcttcatggtcttgaaccaaagtcatttccacccaagggagagtcaggtgaaagtccccagggccctctctaggggaccggagacctccagactaagctggtggaggatgggctcaacctccatgagagaagagcagccaggatcagggggcattaacgttaattttcccaggacttttctgcaaatgggtattggatggaaatatttgttctcagtcagatgagtttctcctattttagtgagaccaaagaaagacaattttaattgtgtccaagctgacttttttgaatgctctggaaatgttggaattccacatcaaagtacgtaactgttttaaactgataactaacccaatatgtgaaaatatatgcaagcatgaataaagggttgactaattccagaattagcaataattttctcttaaatagcaaatttctaaagctgtatgattctctttgcaagaatgtttttcacactgcttaataagaccagttaatgtgtaaaacagaaaaaagtatatatatatcatatgtcttttcatgcatctgaaactttaactgtctatagggttgcttgtcatagttgaacattatttaattaacttattgactatatatgggtatacttttctctatagccattcacttttttaaagttttaattatttaattgacaaattagctattcattttccttcaaatcctgtttttatcacaatgtccattttacagctagacacaaaatttagtgggtccaaattgttcaatcacttctatgttatttgttcagaatagtggatctttgcttaattcacttctgttttaatcccatcatattcctttaggccaagaaaaggaaagctgatttggattatatttatgcttaatatcaacatggtttataagtggacagaaaaacactcactcacgtattcagccacgtatggtctggagtgctctgtagaacagcccgaagtgtacaccatgtctctgcacttgaagctcatggaatgtgttggaggagactcaagctccatgtgggaacagtgtggcccaagagccagcatggaggagggctgtgtgagcagactgctatagaatgctaaagttataatcctagctggtgtctcgttctgtttaaaaaaatcaaatttctgtatgtaattgacgtattggtccttatagtcagtaccatcaggtcttagattgttaagtcattttgctgccaccagaccagtgagagtcactcacttatttgtaatgattcttgggaagtttagtcaagagaatatccttgaataaagaagtacatgttttaagtattttcatcgtagtctagatgggctgtaaaacccatttccacacgagtataaatttaaaacagaaacatcaaggtgtcagcaatcatgattttgttttgcttgttcacaagtttgaaaaggtgcatgaggcaccaatcagtgacactggaatgctttaaggatggtttgtgactttaccccattgtgccttatcatttgtcagcaaacttactgggccaaacacaaatggctgagacactcctggcccatttcttgtcatcgctgccatccccaaagacagactctgggaaacaacttgggaactgcttcaagtccatgccaggtcatggcttcgtccgcctcccagcatgtacctggacttcctttgggtgccggcttttctgctggactaagattcatggaaagaaccccagggcaaggtgaggagaagcagctggtacagactgatgacgaaggagaggaccagaaaagctgcttgggtgtggtggaagtttcagtgtaatgtgattcctagtaggcacatctgtgactcccttaaataaaaagggccagagatgaggggacgcctggtgaaaatggtgtttgttgtaacatcttggtatcattgtgggtaagtagaacaaatatttacatctgttatgaaaagctacctttagccgtagataaacattaaaaggaagactcagccccagagtatatttgatctattggttaactcatctgtcagcaaatggttaagtaagtaaaattaatttttccatttggtgtgtatttcacagggtaacaatgaagtgctccaatctgcttgtgagaccagttagattagtagttgcagttgagaccattcctcctgtatgtctttacaagcaaattgaaacgacatgctcttctttgtactatacagaaggacacaccacagatcctctttttaaagcaggtaataaagtagctttttccttttgaaacatcattgtaaaacagtgctttttaaactatccatggtaaaggatgagttttaaaaattctaatcctttgcaggttggtagttttataaatacgataaaaatatagcaccaagattagattgttataaaagtttctaaacactttcagtttctgtacttaatcttgcagtgtggtaataaagacttctacactttagtagcactaaaa

SEQ ID NO: 29 - DNA, 4579 bp, Homo sapiens
  source 1..4579: mol_type = other DNA
agtccgaccctcggtcccgccgtgtgagcagctggtggagtggagctcagcgcggacgccggagctgcggccgccccctctgcaggtgcctgtgaggaggcgcccgggccgcaaccgctttccgagatccccagtcgcgcactcgctccccgcgctcctgaggggccgcccggccggaggaggccgtcgcggggctcggcgttcagcatgaaccgcagccaccggcacggggcgggcagcggctgcctgggcactatggaggtgaagagcaagtttggagctgaatttcgtcggttttcgctggaaagatcaaaacctggaaaatttgaggagttttatggattactacaacatgttcataagatccccaatgttgacgttttggtaggctatgcagacatccatggagacttactacctataaataatgatgataattatcacaaagctgtttcaacggccaatccactgcttaggatatttatacaaaagaaggaagaagcagactacagtgcctttggtacagacacgctaataaagaagaagaatgttttaaccaacgtattgcgtcctgacaaccatagaaaaaagccacatatagtcattagtatgccccaagactttagacctgtgtcttctattatagacgtggatattctcccagaaacgcatcgtagggtacgtctttacaaatacggcacggagaaacccctaggattctacatccgggatggctccagtgtcagggtaacaccacatggcttagaaaaggttccagggatctttatatccaggcttgtcccaggaggtctggctcaaagtacaggactattagctgttaatgatgaagttttagaagttaatggcatagaagtttcagggaagagccttgatcaagtaacagacatgatgattgcaaatagccgtaacctcatcataacagtgagaccggcaaaccagaggaataatgttgtgaggaacagtcggacttctggcagttccggtcagtctactgataacagccttcttggctacccacagcagattgaaccaagctttgagccagaggatgaagacagcgaagaagatgacattatcattgaagacaatggagtgccacagcagattccaaaagctgttcctaatactgagagcctggagtcattaacacagatagagctaagctttgagtctggacagaatggctttattccctctaatgaagtgagcttagcagccatagcaagcagctcaaacacggaatttgaaacacatgctccagatcaaaaactcttagaagaagatggaacaatcataacattatgaaaccgtggtttgaatgttttcagagtgaggatgccatgaggacttgtacatttggctagtttaaaagcatatatacctctgaccagtgacgtggaataggcatgagacgagtaacgttgcaagcttacaatattattaaagtagtagtttgataattgttaatataaactttggtggatcagaggtgaatttaagtccaaaacaaaggggcctttgctgatgaagttacgtgcttttgctgttttgtctgtggagaatcagatgttaaagcacattcttggaactatgtgagaagactagatcatttctgttggaagtggttgcatatttaacctgctgtgcagagcccagttaatttttcctttaactgtatttttaaaattctaatgtgaagtctgattctctcttgtggtacattggggacctcagctcttaaaggtctcatgttcccaatattttattttgattttttttttttttttttttttttttttttttagtgactgggtctcactctgttgcccacactggaatgcagtggcatgatcacagctctctgcagcctcaatcccctgggctcaagcagtcctcccacctcagcctcctgagtagctgggaccataggcacataccaccacatctgtctactttttgtattttttgtagagacagggtttcgccatgttgcccaagttggtcttgaactcctgggcttaagcagtcctgcctcggcttcccaaaatgctaggattagagccaccatgcccagcctattttgatttttgtttttttatgttcctttctaataaattgtaacaaatgatgttctcaagtacatttccagtttcttttcttttctttctttttttttttttttttttttttgagatggagtctcgctctatcgcccaggctggagtgcagtggcgcgatcttggctcactgcaagctctacctcctgggttcacaccattctcctgcctcagcctcccaagtagctgggactacaggcgcctgccaccatgcctggctaatttttagtagagacggggtttcgcagtgttagccaggaaggtctcaatctcctgacctcctgatccgcccgcctcggcctcccaaagtgctgggattacaggcgtgagccaccgcgcccagttgtgcatttctggtttctaagaatcaaaccacttggctgtttttaggagttacttcccatgttataaagctgaggaagctttttttttttttttttgagacagagtctctgtcacccaggctggagtgcagtggtgcaatctcagctcccgggttcaagcaattctcctgcctcagccttctgagtagctaagattacaggtgtgcgccaacacgtctggcttatttttttgtatttttagtagagatggagtttcaccatgttggtcaggtgggtctcaaactcctgacctcaagtgatccgcccatctcctcccaaagtgctggattgcaggcatgagcgcctagccaggaagctatcttttcttgagttatgaaactttgcaacagttgttcaaattggtgtttgtccttcctatagctttcatattttcaaattaattctgtatggctatataatttatgttttaaaaggcaattctcttgactttggaaatatggaagtctctcctttaacctattcttgttcccattcccagtctcatttgaaatcattccttttattgttagtgtgtgtatttttgttggtgtgcttttaatgcatccaagtatgcatcattttggataaaaaatacatccaaattaagatgttttaacacataggacaaacttgtgcactttttatgccaaaaaaaaaaaaaattgggttttccttcatgggatttctagaaacactgcctacactttatgaaaactacatagtattcacctgtgacaggtagagtttatcactattaattttatgaggctatttattactttccaatgcatccacttagaacaagctaagagtaaggctgctaactttaattccttgcctgattttattgtacagtgtgcacaagcacaatggtatgcttgtatatagaaactaaaaatactatgaagtacataagttccctatggcttatggagagttatttattaattaactttatggtagggctagtatgaatacctttttaacaattgtgtgctattacaacaatgaagattcaaatgactccgctttgaaggatgttttctctatatggtaaaatatatatgaagaagtcttgattacgtgaagatcacttgactcagaatacttcaatgtattttgttcacattaccactaagcatattatcagtaaactattaactgactgcacattatgtaatacgttgtactttttgttgaattcaccgaagttcttccatttatatgctatttttaatggcattccggctttaacattctgtgagtcttacaaatttgactcttgaatggcaaaataatgttagtatgtagaaggttaactttcatttataatataagtggtgcaggggttcaacattttaagtaaaaatatttttacacactacctctctctttttttttttaaagttttaacatcagaacttttgggggaaaaactacttcagggcttgactttttgtacaaattttaactgtaaaatacagatttatcttgtacgcattcatggaaatggaaatcaaagctgctattgctttttattttaattatcctgttaagggtatctatcaatggtattttcaagtagatctctgtttcttaaattattggtgaaataattgattactagatatattgtaaaaccaatagatcctggttatacgataaaatatcagctcattggtaggctgaatcaattatttcaagtgcaccttattaacaaaagtatcagtggatccaacataaaattttatagtactaaatgtcaagcctaactgtgaattttgttctgtatcttaagtaaatttatgataatgttctcgagctatcaacaaaatatatgtacttttgtgagctatgaattttctaattaaattttacatgctataacatgatttttacatgaatgatactttgtttataactatcaaatgtcagtattttactacaattttattataaagtgtacattatcactaaatgaacttcgattttaaaaatcaaattagctttagttgtatattattttttacaaataaagatagacttgtataaaggcta

SEQ ID NO: 30 - DNA, 3873 bp, Homo sapiens
  source 1..3873: mol_type = other DNA
atgagtggccccctagaaggggctgatgggggaggggaccccaggcctggggaatcattttgtcctgggggcgtcccatcccctgggcccccacagcaccggccttgcccaggccccagcctggccgatgacaccgatgccaacagcaatggttcaagtggcaatgagtccaacgggcatgagtctagaggcgcatctcagcggagctcacacagctcctcctcaggcaacggcaaggactcagccctgctggagaccactgagagcagcaagagcacaaactctcagagcccatccccacccagcagttccattgcctacagcctcctgagtgccagctcagagcaggacaacccgtccaccagtggctgcagcagtgaacagtcagcccgggcaaggactcagaaggaactcatgacagcacttcgagagctcaagcttcgactgccgccagagcgccggggcaagggccgctctgggaccctggccacgctgcagtacgcactggcctgtgtcaagcaggtgcaggccaaccaggaatactaccagcagtggagcctggaggagggcgagccttgctccatggacatgtccacctataccctggaggagctggagcacatcacgtctgagtacacacttcagaaccaggataccttctcagtggctgtctccttcctgacgggccgaatcgtctacatttcggagcaggcagccgtcctgctgcgttgcaagcgggacgtgttccggggtacccgcttctctgagctcctggctccccaggatgtgggagtcttctatggttccactgctccatctcgcctgcccacctggggcacaggggcctcagcaggttcaggcctcagggactttacccaggagaagtccgtcttctgccgtatcagaggaggtcctgaccgggatccagggcctcggtaccagccattccgcctaaccccgtatgtgaccaagatccgggtctcagatggggcccctgcacagccgtgctgcctgctgattgcagagcgcatccattcgggttacgaagctccccggataccccctgacaagaggattttcactacgcggcacacacccagctgcctcttccaggatgtggatgaaagggctgcccccctgctgggctacctgccccaggacctcctgggggccccagtgctcctgttcctgcatcctgaggaccgacccctcatgctggctatccacaagaagattctgcagttggcgggccagccctttgaccactcccctatccgcttctgtgcccgcaacggggagtatgtcaccatggacaccagctgggctggctttgtgcacccctggagccgcaaggtagccttcgtgttgggccgccacaaagtacgcacggcccccctgaatgaggacgtgttcactcccccggcccccagcccagctccctccctggacactgatatccaggagctgtcagagcagatccaccggctgctgctgcagcccgtccacagccccagccccacgggactctgtggagtcggcgccgtgacatccccaggccctctccacagccctgggtcctccagtgatagcaacgggggtgatgcagaggggcctgggcctcctgcgccagtgactttccagcagatctgtaaggatgtgcatctggtgaagcaccagggccagcagctttttattgagtctcgggcccggcctcagtcccggccccgcctccctgctacaggcacgttcaaggccaaggcccttccctgccaatccccagacccagagctggaggcgggttctgctcccgtccaggccccactagccttggtccctgaggaggccgagaggaaagaagcctccagctgctcctaccagcagatcaactgcctggacagcatcctcaggtacctggagagctgcaacctccccagcaccactaagcgtaaatgtgcctcctcctcctcctataccacctcctcagcctctgacgacgacaggcagaggacaggtccagtctctgtggggaccaagaaagatccgccgtcagcagcgctgtctggggagggggccaccccacggaaggagccagtggtgggaggcaccctgagcccgctcgccctggccaataaggcggagagtgtggtgtccgtcaccagtcagtgtagcttcagctccaccatcgtccatgtgggagacaagaagcccccggagtcggacatcatcatgatggaggacctgcctggcctagccccaggcccagcccccagcccagcccccagccccacagtagcccctgacccagccccagacgcctaccgtccagtggggctgaccaaggccgtgctgtccctgcacacacagaaggaagagcaagccttcctcagccgcttccgagacctgggcaggctgcgtggactcgacagctcttccacagctccctcagcccttggcgagcgaggctgccaccacggccccgcacccccaagccgccgacaccactgccgatccaaagccaagcgctcacgccaccaccagaaccctcgggctgaagcgccctgctatgtctcacacccctcacccgtgccaccctccaccccctggcccaccccaccagccactacccccttcccagcggttgtccagccctaccctctcccagtgttctctcctcgaggaggcccccagcctcttccccctgctcccacatctgtgcccccagctgctttccccgcccctttggtgaccccaatggtggccttggtgctccctaactatctgttcccaaccccatccagctatccttatggggcactccagacccctgctgaagggcctcccactcctgcctcgcactccccttctccatccttgcccgccctccccccgagtcctcctcaccgcccggactctccactgttcaactcgagatgcagctctccactccagctcaatctgctgcagctggaggagctcccccgtgctgagggggctgctgttgcaggaggccctgggagcagtgccgggcccccacctcccagtgcggaggctgctgagccagaggccagactggcggaggtcactgagtcctccaatcaggacgcactttccggctccagtgacctgctcgaacttctgctgcaagaggactcgcgctccggcacaggctccgcagcctcgggctccttgggctctggcttgggctctgggtctggttcaggctcccatgaagggggcagcacctcagccagcatcactcgcagcagccagagcagccacacaagcaaatactttggcagcatcgactcttccgaggctgaggctggggctgctcggggcggggctgagcctggggaccaggtgattaagtacgtgctccaggatcccatttggctgctcatggccaatgctgaccagcgcgtcatgatgacctaccaggtgccctccagggacatgacctctgtgctgaagcaggatcgggagcggctccgagccatgcagaagcagcagcctcggttttctgaggaccagcggcgggaactgggtgctgtgcactcctgggtccggaagggccaactgcctcgggctcttgatgtgatggcctgtgtggactgtgggagcagcacccaagatcctggtcaccctgatgacccactcttctcagagctggatggactggggctggagcccatggaagagggtggaggcgagcagggcagcagcggtggcggcagtggtgagggagagggctgcgaggaggcccaaggcggggccaaggcttcaagctctcaggacttggctatggaggaggaggaagaaggcaggagctcatccagtccagccttacctacagcaggaaactgcaccagctag

SEQ ID NO: 31 - DNA, 6380 bp, Homo sapiens
  source 1..6380: mol_type = other DNA
cgtaaccgccgccggcgcgcgggcctcgggcaggtcggggtcccagcgccggctcgggcagcggaggcgccgccggaagttccttgggctgctggactcctcggcttgaaacggcgccggcgtgggggcgtgtgcccttggccctgtcccaggtggagagtggtcgagccgcgcgcagggtgcgctcgtttgaactgcggtgacaccgagggttggggactcgaacccccgcttcgcagctcaggagcctgaggtccgaaagcttcgttccagagcccagcatgaatggatacgcggaatttccgcccagccccagtaaccccaccaaggagcccgtggagccccagcccagccaggtcccactgcaggaagatgtggacatgagcagtggctccagtggacatgagaccaacgaaaactgctccacggggcgggactcgcagggcagtgactgtgacgacagtgggaaggagctggggatgctggtggagccaccggatgcccgccagagtccagatacctttagcctgatgatggcaaaatctgaacacaacccatctacaagtggctgcagtagcgaccagtcttcgaaagtggacacacacaaagaactgataaaaacactaaaggagctgaaggtccacctccctgcagacaagaaggccaagggcaaggccagtacgctggccaccttgaagtacgccctcaggagcgtgaagcaggtgaaagccaatgaagagtattaccagctgctgatgtccagcgagggtcacccctgtggagcagacgtgccctcctacaccgtggaggagatggagagcgttacctctgagcacattgtgaagaatgccgatatgtttgcggtggccgtgtccctggtgtctgggaagatcctgtacatctctgaccaggttgcatccatatttcactgtaaaagagatgccttcagcgatgccaagtttgtggagttcctggcgcctcacgatgtgggcgtgttccacagtttcacctccccgtacaagcttcccttgtggagcatgtgcagtggagcagattcttttactcaagaatgcatggaggagaaatctttcttttgccgtgtcagtgtccggaaaagccacgagaatgaaatccgctaccaccccttccgcatgacgccctacctggtcaaggtgcgggaccaacaaggtgctgagagtcagctttgctgccttctgctggcagagagagtgcactctggttatgaagcccctagaattcctcctgaaaagagaatttttacaaccacccatacaccaaattgtttgttccaggatgtggatgaaagggcggtccctctcctgggctacctacctcaggacctgattgaaaccccagtgctcgtgcagctccaccctagtgacaggcccttgatgctggccatccacaaaaagatcctgcagtcaggcgggcagcctttcgactattctcccattcggtttcgcgcccggaacggagagtacatcacgttggacaccagctggtccagcttcatcaacccatggagcaggaaaatctccttcatcattgggaggcacaaagtcagggtgggccctttgaatgaggacgtgtttgcagcccacccctgcacagaggagaaggccctgcaccccagcattcaggagctcacagagcagatccaccggctcctgctgcagcccgtcccccacagcggctccagtggctacgggagtctgggcagcaacgggtcccacgagcaccttatgagccagacctcctccagcgacagcaacggccatgaggactcacgccggaggagagccgaaatttgtaaaaatggtaacaagaccaaaaatagaagtcattattctcatgaatctggagaacaaaagaaaaaatccgttacagaaatgcaaactaatcccccagctgagaagaaagctgtccctgccatggaaaaggacagcctgggggtcagcttccccgaggagttggcctgcaagaaccagcccacctgctcctaccagcagatcagctgcttggacagcgtcatcaggtacttggagagctgcaatgaggctgccaccctgaagaggaaatgcgagttcccagcaaacgtcccagcgctaaggtccagtgataagcggaaggccacagtcagcccagggccacacgctggagaggcagagccgccctccagggtgaacagccgcacgggagtaggtacgcacctgacctcgctggcactgccgggcaaggcagagagtgtggcgtcgctcaccagccagtgcagctacagcagcaccatcgtccatgtgggagacaagaagccgcagccggagttagagatggtggaagatgctgcgagtgggccagaatccctggactgcctggcgggccctgccctggcctgtggtctcagccaagagaaggagcccttcaagaagctgggcctcaccaaggaggtactcgctgcacacacacagaaggaggagcagagcttcctgcagaagttcaaagaaataagaaaactcagcattttccagtcccactgccattactacttgcaagaaagatccaaggggcagccaagtgaacgaactgcccctggactaagaaatacttccggaatagattcaccttggaaaaaaacaggaaagaacagaaaattgaagtccaagcgggtcaaacctcgagactcatctgagagcaccggatctggggggcccgtgtccgcccggcccccgctggtgggcttgaacgccacagcctggtcaccctcagacacgtcccagtccagctgcccagccgtgccctttcccgccccagtgccagcagcttattcactgcccgtgtttccagcgccagggactgtggcagcacccccggcacctccccacgccagcttcacagtgcctgctgtgcccgtggacctccagcaccagtttgcagtccagcccccacctttccctgcccctttggcgcctgtcatggcattcatgctacccagttattccttcccctcggggaccccaaacctgccccaggccttcttccccagccagcctcagtttccgagccaccccacactcacatccgagatggcctctgcctcacagcctgagttccccagccggacctcgatccccagacagccatgtgcttgtccagccacccgggccaccccaccatcggccatgggtagggcctccccaccgctctttcagtcccgcagcagctcgcccctgcagctcaacctgctgcagctggaggaagcccctgagggtggcactggagccatggggaccacaggggccacagagacagcagctgtaggggcggactgcaaacctggcacttctcgggaccagcagccgaaggcgcctctgacccgtgatgaaccctcagacacacagaacagtgacgccctttccacgtcaagcggcctcctaaacctcctgctgaatgaggacctctgctcagcctcgggctctgctgcttcggagtctctgggctccggctcactgggctgcgacgcctccccgagtggggcaggcagtagtgacacaagtcataccagcaaatattttggaagcattgactcctcagagaataatcacaaagcaaaaatgaacactggtatggaagaaagtgagcatttcattaagtgcgtcctgcaggatcccatctggctgctgatggcagatgcggacagcagcgtcatgatgacgtaccagctgccttcccgaaatttagaagcggttttgaaggaggacagagagaagctgaagctcctacagaaactccagcccaggttcacggagagtcagaagcaggagctgcgcgaggtccaccagtggatgcagacgggcggcctgcccgcagccatcgacgtggcagaatgtgtttactgtgaaaacaaggaaaaaggtaatatttgcataccatatgaggaagatattccttctctgggactcagcgaagtgtcggacaccaaagaagacgaaaatggatcccccttgaatcacaggatcgaagagcagacgtaacccctgccccacctcagcccggcagccagcgaggtacaccaggtggtgcttggaagagatgaaagatcttcatggctgtttccactgaaatggacacatatgctcatgttgctttttttgttttagaaaaaaaaacaacatagttttctgaaggggcgacttaaaactgtggagagtggggagagttcggaaagaaatatgtttttatatataaaatatatatgtggagttttgtgggatggggaagagattttagttgttatttaacttgagaaagactaagcgcctcttagtgtcagggaagttgcctcagtgctcccagaagtcctgtgactgtgacgagacctctgtctgctgcaccagctggggactctggcttccagagctttcccagggtgtttggatcagatcaaattttgtcctctcttggggactgctttttatctgaattatcatttagtcaaggtagagtgtttttttatacataccaaatggagatagcagcctctcctagttttatttcaaaacgtttcacattaaatggtgtgaagcgttgtttggcaaaccaacagctttggcttctggtgtggtcaatatttcagtctgacataggttttgtttgtagtgaacaaagttgaaacatttgctctggactaaagaagcctagtggtttgtgtggccaactccatcggatgaatgcacacgcagacagaccctctgtatatttctgcattattcttgtctccttttcagaccatgatggccaatatggagattaaaatatgtcatcagtcatctctttatggtgacttccctttgcaaaccaggctgtgaccaacacatgtgagacccagtcctgtttggttttcttccgttggaaccacccagacatctgcttccacccagccaagcccacatcacatctcctggccgagagcagccactgccactcagtctgacagcttgcgactgcatctgtattttcaggggtgcagtgagctcacctctcccactgcaccctgggttgggtgcacagccctcattcttttcatgagcccgacctctctcggagcagcttcaggcctctgccagtgtccccagcacttttaggtcatttggacacttggggaaaagtgaggccagtctgcccggctttttacaaaacctcatgttgcattgtatattccaaagatggttcagaaaatttaatattggtccctggtggaaattcaaagttatcactgaagaacagttgacttaaaattggaccaagactatgaggcttaaaagggaccagggttttcttttttttttttttttttttttttttttagatggagtttctttttgcccaggctggagtgcagtggcgccatcttggctcactgcaacctctgcctcccaggttcaagcgattctcctgcctcagcctcctgagtagctgggaccacaggcgactgccaccacacccagctaattttttgtatttttagtagagacagggtttcaccatgttggccaggctggtctcgaactcctgacctcaagcgatccacccacctcggcctcccaaagtgctgggattacaggcgtgagccaccacgcccaactgggaccagggttttctgttttttgatggaggtgaaatctctttgtaatccactaggttttcatcgtaaaaccatcttatgcctgactattaaacctattcttcataaacacaagaacactttaatttttcgttaatttacaaagtaacatcagctgcctatgcctatgataaggtagcagtctgcattcttatggccattagatgttacaaactccttgcctctaaagtcagatcatgaagggataggtgttcatctaaggttacagttatgttaccgaaacacaaaactgccaaaatcttactctgctgttatgaatgtttaccatcagcattattttatcatttaatatgtgctcactgattgttaactgtagcttcagcgcgtgccaagcagttgacttaataggatcatcttgtgaatttgtttacgtgatgccaagcatcaagtcatgttttctttagtgtgtgtgcttacacaggtgttaaacagtttttctctattttaaactgagccttctttttaatatattcccgaagagatatgtaaataagctctcagagtttctgtgatgatttgttgagccttgctggacaagtggtttgtttgtgtgcaaaccaaactttctttacccagtgcaatagatttgtttgactgcttgtgtctttttatgacctgtttgccttttagaaaattggtaaataaagcaagtatattttta

SEQ ID NO: 32 - DNA, 6430 bp, Homo sapiens
  source 1..6430: mol_type = other DNA
gaccggcacgcggcgagcctcgagactgcgcgagggcggccccgggggcgagcggctgtgcgcggggccaagggcgggggcagcaggtgagtgcgcggccgggcgggagttctgggcggccgggcgggagttctgggcactcgcgcgcggcagaggagccccgcgagccctgccctcggccggagtcctgaaagtcgagcgagctccgggttttgaaaatgttggagggaaaagctcctcggagatgagcgtgaccccctggctcgtggtggccgcctgttctcactaacgccatggcggggaccggagtgagaaaccggtgtctgtcactgactgcaaagtgagcgagaagcaggctgcgggccgtcccagcacgacgtggagccccgcggagacctcgagatgccccgcggggaagctcctggccccgggagacggggggctaaggacgaggccctgggcgaagaatcgggggagcggtggagccccgagttccatctgcagaggaaattggcggacagcagccacagtgaacagcaagatcgaaacagagtttctgaagaacttatcatggttgtccaagaaatgaaaaaatacttcccctcggagagacgcaataaaccaagcactctagatgccctcaactatgctctccgctgtgtccacagcgttcaagcaaacagtgagtttttccagattctcagtcagaatggagcacctcaggcagatgtgagcatgtacagtcttgaggagctggccactatcgcttcagaacacacttccaaaaacacagatacctttgtggcagtattttcatttctgtctggaaggttagtgcacatttctgaacaggctgctttgatcctgaatcgtaagaaagatgtcctggcgtcttctcactttgttgacctgcttgcacctcaagacatgagggtattctacgcgcacactgccagagctcagcttcctttctggaacaactggacccaaagagctgcacggtatgaatgtgctccggtgaaaccttttttctgcaggatccgtggaggtgaagacagaaagcaagagaagtgtcactccccattccggatcatcccctatctgattcatgtacatcaccctgcccagccagaattggaatcggaaccttgctgtctcactgtggttgaaaagattcactctggttatgaagctcctcggatcccagtgaataaaagaatcttcaccaccacacacaccccagggtgtgtttttcttgaagtagatgaaaaagcagtgcctttgctgggttacctacctcaggacctgattggaacatcgatcctaagctacctgcaccctgaagatcgttctctgatggttgccatacaccaaaaagttttgaagtatgcagggcatcctccctttgaacattctcccattcgattttgtactcaaaacggagactacatcatactggattccagttggtccagctttgtgaatccctggagccggaagatttctttcatcattggtcggcataaagttcgaacgagcccactaaatgaggatgtttttgctaccaaaattaaaaagatgaacgataatgacaaagacataacagaattacaagaacaaatttacaaacttctcttacagccagttcacgtgagcgtgtccagcggctacgggagcctggggagcagcgggtcgcaggagcagcttgtcagcatcgcctcctccagtgaggccagtgggcaccgtgtggaggagacgaaggcggagcagatgaccttgcagcaggtctatgccagtgtgaacaaaattaaaaatctgggtcagcagctctacattgagtcaatgaccaaatcatcattcaagccagtgacggggacacgcacagaaccgaatggtggtggtgaatgtaagacctttacttccttccaccaaacactgaaaaacaatagtgtgtacactgagccctgtgaggatttgaggaacgatgagcacagcccatcctatcaacagatcaactgtatcgacagtgtcatcagatacctgaagagctacaacattccagctttgaaaagaaagtgtatctcctgtacaaatacaacttcttcctcctcagaagaagacaaacagaaccacaaggcagatgatgtccaagccttacaagctggtttgcaaatcccagccatacctaaatcagaaatgccaacaaatggacggtccatagacacaggaggaggagctccacagatcctgtccacggcgatgctgagcttggggtcgggcataagccaatgcggttacagcagcaccattgtccatgtcccacccccagagacagccagggatgctaccctcttctgtgagccctggaccctgaacatgcagccagcccctttgacctcggaagaatttaaacacgtggggctcacagcggctgttctgtcagcgcacacccagaaggaagagcagaattatgttgataaattccgagaaaagatcctgtcatcaccctacagctcctatcttcagcaagaaagcaggagcaaagctaaatattcatattttcaaggagattctacttccaagcagacgcggtcggccggctgcaggaaagggaagcacaagcggaagaagctgccggagccgccagacagcagcagctcgaacaccggctctggtccccgcaggggagcgcatcagaacgcacagccctgctgcccctccgcggcctcctctccgcacacctcgagcccgaccttcccacctgccgccatggtgcccagccaggccccttacctcgtcccagcttttcccctcccagccgcgacctcacccggaagagaatacgcagcccccggaactgcaccggaaggcctgcatgggctgcccttgtccgagggcttgcagccttacccagctttcccttttccttacttggatacttttatgaccgttttcctgcctgacccccctgtctgtcctctgttgtcgccatcgtttttgccatgtccattcctgggggcgacagcctcttctgcgatatcaccctcaatgtcgtcagcaatgagtccaactctggacccacccccttcagtcaccagccaaaggagagaggaggaaaagtgggaggcacaaagcgaggggcacccgttcattacttcgagaagcagctcacccttgcagttaaacttacttcaggaagagatgcccagaccctctgaatctccagatcagatgagaaggaacacgtgcccacaaactgagtattgtgttacaggcaacaatggcagtgagagcagtcctgctactaccggtgcactgtccacggggtcacctcccagggagaatccatcccatcctactgccagcgctctgtccacaggatcgcctcccatgaagaatccatcccatcctactgccagcgctctgtccacaggatcgcctcccatgaagaatccatcccatcctactgccagcacactgtccatgggattgcctcccagcaggactccatcccatcctactgccactgttctgtccacggggtcacctcccagcgaatccccatccagaactggttcagcagcatcaggaagcagcgacagcagtatataccttactagtagtgtttattcttctaaaatctcccaaaatgggcagcaatctcaggacgtacagaaaaaagaaacatttcctaatgtcgccgaagagcccatctggagaatgatacggcagacacctgagcgcattctcatgacataccaggtacctgagagggttaaagaagttgtactaaaagaagacctggaaaagctagaaagtatgaggcagcagcagccccagttttctcatgggcaaaaggaggagctggctaaggtgtataattggattcaaagccagactgtcactcaagaaatcgacattcaagcctgtgtcacttgtgaaaatgaagattcagctgatggtgcggccacatcctgtggtcaggttctggtagaagacagctgttgagtgactgtgaggatgaaccttcataccctttccaagacgtgttacacagacagacctttttaagtcctggacttttaaatgaccatgaagttatcattgaatgttaagattttttcttcttgattttttaatacacgtaatctttttgaagcagacattgtatacagaatcttacttctctttgttcctgatatattaaaatggccagttaggctctttttgtagttgaattgtcttctaaagagattggatggcctctaaagaggtatgtgtatctttatttcagatgtcacccagagtaaattataattagaagtatagctagaatgagccccaaaccttagcctcatttattttgttctgttacataagtcattttccccttagagtgcttgaagaaatgccacctacaggttgtgtacttttcataatggtttccatgaatgtagtacgttcatacaggcttcattcaacctggcgttcccctccataattaagatgaaacattccggttttctcacaacacattagcacatactgtccattagcatatctgggataaccaggttttgggggttgagttttggccttcatccttgtagatccctttcctattgatttcccaccttccagtgaaattctgaaagtcttatcttaaaaatcgatccgcttaccatgggcctattcttgtaagtttcagttagcatttgcatgtgtaatattaaaatgaaagagcttcttacccagtgctgttgcccttttgagtatttttgtttttaaaataatgattgtaaaatgttttacaagtaatgtaaaagctagtatcattcttacatacttctgtgtttaaattttcattcttaccaaaacagttaactctttctttccaatcaatttatacaaaagaggtcgctccagccctaccacaggtctgactggcactgccttttgtttgcccttgaacagggcagtgttgtggggactgcaaaagagaaaacgtccaggcgagcccagttgtcctcgcccacagggtcctgcaggctccatcagtcaccgctttctatggcgtttgtagttgtgtcttttaagaagtgagtgtgattgtttacttgataaatcagctcactctctggtgctttttagagaagtccctgattccttcttaaacttggaatgatagatgaaattcacacccctgcagatcagaaaaaacaaatagaagaaaatgagggttacagtaacctgttgtctttatataacttgcaacaaactaatttatttttttttcctttttttgtttttggttttttatggttttttaaggaaaatacttttctcctttgaagttttacagctttttgtaaatgcgtcctgataatgattaggaaaatcgaccttttcatccatgatgaccatcctcatagctcagatctcctttcaaagtagtggctttctggatggtaattccatcttaaggtgtcagaactattttcaaatgctgcctttgacagttcttggaattttctgatattaagcagttccatgcaaatattcgtgttttataaatagctctcatagtctgctccatcttgatagttaagtgatttctgaagcgtttgtgtgtgtgttgatcaggttgtgtgatatttttgcttgatagagaatcaaatttgaaacaattaaccagccagtagattgtctgtcagtgaccttctgtagtaataaagtttttgccactgtaaataaaaacagtatccgtagctatcaggatcattgcgcactcatatatgctaagccttctgttctctaatagaagcctttcttttccattgtttctggatatttgtattatccaaatgtgcttatttctttgccttagcacacgttttatggagtacttgttatactaggtttgatttgaaactggtgcttgtcgcagaactgtcagagcatgaggagcgctcctcctgtgggtggacgcattcacgcactcccaggttgcacctgctgctggcggtgagcagggggttcagcagcttgaccgatgccccccgagggggctctccccagcttaaactttgttgtttaaatttgttaactttttatattaatgactattgaaagtggtaataaaaatttatattataggcttcaatgttttcatgaatgttacccaaaaagctgtgttttctttggtcagaggtcaaaatttatgaaaaacaaaatgctgtatgaatggaaatcattttgcaattgagtgacacttcattgtaattcacagtgtaaatttaatccaaactgaaattttgtttcaactgaatttgtaattaactctgaatttgtttttaatcattagtaatatttcagttgggtatctttttaagtaaaaacaacaaataaactctgtacatgtaaaacgtga

SEQ ID NO: 33 - DNA, 1409 bp, Homo sapiens
  source 1..1409: mol_type = other DNA
gagtagtcccttcgcaagccctcatttcaccaggcccccggcttggggcgccttccttccccatggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaactgaggtgcctgcccttctaggaatgggggacagggggaggggaggagctagggaaagaaaacctggagtttgtgccagggtttttgggattaagttcttcattcactaaggaaggaattgggaacacaaagggtgggggcaggggagtttggggcaactggttggagggaaggtgaagttcaatgatgctcttgattttaatcccacatcatgtatcacttttttcttaaataaagaagcctgggacacagtagatagacacactta

SEQ ID NO: 34 - DNA, 939 bp, Homo sapiens
  source 1..939: mol_type = other DNA
gttgttgtgtcccagccttcccaagcttccaggtgtcccagaaacccaggaaatcgagactcatgactcccagagaggatggcatctagaagtagacgatcaagggtggaatctacagtccgtgagccctgacttcttgccttcgtctcaaatagactctgcagccagccatctatgcagcgccccagtggctttgaaatgcaacagaaaccatcacccccggaccgtgggctccatgccagtgggcaaagcacagcctgggaagaattggtttgcagccaggcagtcctccatccagtcttgactttggcacttgtgatatgacttgcacagaccaaaaaagtcactctcaaagagctctcggcacacagaccccagctttacaaggaccccagctccttaacacagatcccagctccgaggaaactcgtcccccccacgttaatcctgaccgactttgccacatggagccagcaaaccatttctggcatgcaggggacctccaagcaatgatatccaaggaattccatctggcagccacccaggatgactgcagaaaaggaaggacacaggaggatatcctggttccctcttcccacccagagctgtttgcatcagtcctgccaatggctccggaagaagctgccaggctccagcaacctcagccccttcctcctccctcaggaatccacctatccgcctctaggaccttggctccaactctattgtactcgtctcctccctcccattctccttttggtctcagctccttgatctaagcctcccagagagacccctagaacgtttccctcaaggacctttctgcctggaagtctgttagcctttcagaagtaacatgtccaaaataaaatttgattcctcccaggttgttccctgcctggtccgctaccccacagtaaggaacaccttattatgcaa

SEQ ID NO: 35 - DNA, 1143 bp, Homo sapiens
  source 1..1143: mol_type = other DNA
gagatgccaagtgggtattgactgctccaggatgtggatggagggtgtgaaaaccagggtggggtgacgcaggctctgggtcatgatagggagagcaggcagctgggtcctgggctggaggactaaaataagggacgccaccttcaggggtgacacatcagcccaggccctccctacgggtttgaccagtcctgttctgacggtattcctgcgccactgggctggtccctcctccactcctcccctataaagcctcctggggtccccaggcacccagactcagcccaccccagctttgggggccagtacgcagccatgatcctcaactggaagctcctggggatcctggtcctttgcctgcacaccagaggcatctcaggcagcgagggccacccctctcacccacccgcagaggaccgagaggaggcaggctccccaacattgcctcagggccccccagtccccggtgacccttggccaggggcaccccctctctttgaagatcctccgcctacccgccccagtcgtccctggagagacctgcctgaaactggagtctggccccctgaaccgcctagaacggatcctcctcaacctccccggcctgacgacccttggccggcaggaccccagcccccagaaaacccctggcctcctgcccctgaggtggacaaccgacctcaggaggagccagacctagacccaccccgggaagagtacagataatggagtcccctcagccgttctgttcccaggcatctccaggcacccacgccctctccaccctctgattccccgtgaattcttcccaatttagcctatctccttaaacctcttcctcattccctcggttttattctgaacccgtaaggtggtgttctcaatatttcctgtcccctcctgagatccatacttagtcctcacatcgcccgttttttcctctgacagcctaagcctactctcctacctcgcctccaggcctcggccccacctacctcccacccggtcttcctgcccgcgcgatcgctggggcagggctacggtactgtgttcccttctgccacctggtggccggcggcaggaactatcagtagacagctgctgcttccatgaaacggaaaaataaaaatcatgttttcttaa

SEQ ID NO: 36 - DNA, 1202 bp, Homo sapiens
  source 1..1202: mol_type = other DNA
ccggccagcgggcgggctccccagccaggccgctgcacctgtcagtggagggggaggagaggtttggctgccagattcaactggaaaggaaccagtcccagtccagccgcaacctgggagtgggaagctggaggcagcccagacctcctggagccctgcagtcctggggcagagacagaatcaggacacagctcgaggtcagggccagaggctggagctgagggcctagagtgagagggggcaaggcaagggggggagaggaagagaggcaggattagagagaggaggcaggccagaaagaggagagcaggagagacccaaagagaaacagaaggcagatagagagggagtgagaggcaggagctgagacacgagtcctggaggaagaagaccaaaggaagggggcagagacagaaagggaggtgctaggacaaaactcgaaaggtggccctatcagggaagcagaggagaggccgttctagggaagcccagctccggcacttttggccccaactcccgcaggtctgctggctccaggaaaggtggaggagggagggaggagtgggagaatgtgggcgcagggtgggacatgggcatggccaggggcagcctcactcgggttccaggggtgatgggagagggcactcagggcccagagctcagccttgaccctgacccttgctctctccaatccactccggggctcatgaagggaacaagctggaggagcaggaccctagacctctgcagcccataccaggtctcatggaggggaacaagctggaggagcaggactctagccctccacagtccactccagggctcatgaaggggaacaagcgtgaggagcaggggctgggccccgaacctgcggcgccccagcagcccacggcggaggaggaggccctgatcgagttccaccgctcctaccgagagctcttcgagttcttctgcaacaacaccaccatccacggcgccatccgcctggtgtgctcccagcacaaccgcatgaagacggccttctgggcagtgctgtggctctgcacctttggcatgatgtactggcaattcggcctgcttttcggagagtacttcagctaccccgtcagcctcaacatcaacctcaactcggacaagctcgtcttccccgcagtgaccatctgcaccctcaatccctacaggtcggttagtccctctgcccc

SEQ ID NO: 37 - DNA, 5718 bp, Homo sapiens
  source 1..5718: mol_type = other DNA
ggccgagcgcgcggcctggcgcacgatacgccgagccggtctttgagcgctaacgtctttctgtctccccgcggtggtgatgacggtgaaaactgaggctgctaagggcaccctcacttactccaggatgaggggcatggtggcaattctcatcggtgagtgcaggaatcttgcgggacttctgctccaggagacgcaaagtggaaattttttgaaagtcccggatcagattagtgtgtgtggcgccgggacgttatgaagccgtctaaacgtttctttatttctcctccttctatccacagctttcatgaagcagaggaggatgggtctgaacgactttattcagaagattgccaataactcctatgcatgcaaacagtaagttcagaccggattgaggaaataactagtatagtttgaatttgccagcggtaaacattctcatcacggcgtttatcgggaaggcgaagacttcttctggggtggggatctcatttctccttaaattctaatatatttgacacattttaaacattaaagttaatttgctgatttggcttgaactggagatgtaagataaatggttcgtgttggccgaattcacgctttctccatgagcaacaatccttatttctgtatttaatggggtttattattttctttaactgactaatgtattggggtattttcagtttaaacagtgaattatcgggtagaagtcggtagagccagaaactcacttttgatgttggtgtgccccctagtggcgagctggattctaaatcgtgccctttattccctgcagccctgaagttcagtccatcttgaagatctcccaacctcaggagcctgagcttatgaatgccaacccttctcctccagtaagtttttgtatgtgccgtgcatctgtggagaactgtaagggagtcagttagtattcctacattaatggattaaaatagcatttctagaaattagtatcaaggcaggaatgcttcattatgcataacagtgatataaatatttaagtattgagtcagagtattatttttatttttttcctgggcatattttacctcaagtggttattttaaaaggcatatttcataaaaaggttttatctgtctgaaacaacatgactgtgtgcagtttccatactcatttgaaatgtgatgaaatgtagttttgaatgtttatagatgtatggtcatttgcatcagtcatttgtagatgtaacattttctacatcgtttatgttatagatgtcttcctttgaagcaatggtattaaaagaaattctttttttttttttctagccaagtccttctcagcaaatcaaccttggcccgtcgtccaatcctcatgctaaaccatctgactttcacttcttgaaagtgatcggaaagggcagttttggaaaggtaatttcaaatctgaagatcttttggtacacttccttcatgtcctcttttatattctccctggatgaggatcgaaaaatgatttttttaaattgaaatttcaggttcttctagcaagacacaaggcagaagaagtgttctatgcagtcaaagttttacagaagaaagcaatcctgaaaaagaaagaggtgagatgtgcttgatggggctggcattggcggtagacactccttgaataatcttgattctggaatgttggtgccagttgaacatgccactaaatctgaatcgtcattttcctaggagaagcatattatgtcggagcggaatgttctgttgaagaatgtgaagcaccctttcctggtgggccttcacttctctttccagactgctgacaaattgtactttgtcctagactacattaatggtggagaggtgagcaggggggatagaagtcaactcttagtgtctctgcacagcctgctttgttttagtttgagaaaaaagttttcaaagatttttggtggggagaatgttaccagaattagcatttccttcaacctgtcaggttatagttaatagattacttggggccacttcctgcagttgttcttttgctgtgtatgtcaaaactaattaaattacattgcgcaacccagaatgactttgttctgtctcctgcagttgttctaccatctccagagggaacgctgcttcctggaaccacgggctcgtttctatgctgctgaaatagccagtgccttgggctacctgcattcactgaacatcgtttataggtaagcctgagagctcttcaggctaccagttttggtataaaggagacgtagcactggctgtttcatagggccttaaaataatttgtgtttatttgcaacttggttcgctaaaaccagatcccctagcacgtgagctggcttgacttaagtgccaagggggaacagccaagtaggattgtgcctaatccagaatagatgagcagaacaagggctccttttttcttcactacacaactacagtgaacctaaatgcctctaataccttagcaattatctttaagaggatatcttatgaagtgaaattaacttgtgcaactacttttctattcacttttttacagagacttaaaaccagagaatattttgctagattcacagggacacattgtccttactgatttcggactctgcaaggagaacattgaacacaacagcacaacatccaccttctgtggcacgccggaggtaggcgctgtcttggtttggtgcctggtttacccccgccttccaagagagagatgtacaatcatgcacttaactaccaaaaagagtaaactcctctcagagacttcttaatacagttcagtgcaaataaaatacatttgctgtttgatgtagcatgagaaatcccaagtccttctgttcctttactgaaaagtagctgtttgtaagtaagatctgcatcataaaaactttctaatcctaagtaagagatatcaagtgccagcagtttcctaaatgtcagtacacataggtagccagtcaccctcaaaaagtccagcagttttatcaggaaggaatctaaagatatctatcttccaagctggctctgggtctctcagctttttcaaactaaatgtgtggtcgtgggattgcttgctttcgcaggttctaaacgctgtttccctggtctgtttttcagtatctcgcacctgaggtgcttcataagcagccttatgacaggactgtggactggtggtgcctgggagctgtcttgtatgagatgctgtatggcctggtgagtggcacattgggaaccactggaacactgcctgctccctacaatattgccttcacacagcaaaagcagctaagaggcatattggttattttatagttcataagaataatcacttacctggttcttttgtgcatttcacattttactagataggaccacattgaacctgtgtggtggtgaaaaactaccacttattaacatctaccccctaccctccacacacacacacacaaacacacacacgggttgcaaagtagacacttaaatagcaagggaaaagaaagcattgaggtggggagagtttctcaaatcgagcctaatatttattgccgtttatatctttttctctactggtaatgtgtgccatatgaaacttccaattaagtctaaagtaattttccccttctttcagccgcctttttatagccgaaacacagctgaaatgtacgacaacattctgaacaagcctctccagctgaaaccaaatattacaaattccgcaagacacctcctggagggcctcctgcagaaggacaggacaaagcggctcggggccaaggatgacttcgtgagtgatgttttcctgtcctcctgggccggccgggacgtgcactagacctccctgcccttattgaatgcacctgtctaaattaatcttgggtttcttatcaacagatggagattaagagtcatgtcttcttctccttaattaactgggatgatctcattaataagaagattactcccccttttaacccaaatgtggtgagtatctgtctctcttctaagtatagagaagccaagcgatttattttaattcagaattgtctgggggagggttggaaggaatacattggcagatgttttctccataaacctgttattttacctacatagacacatttatcaattcgaagcaccaaaaggcaacaagtgaacattattcttatgtttaactgtgtgtagccttttgagattttgtgcttgaagtgggtgattatggaagttgatataagacttaaacttggtatttaaagcctggtcaagatttccctgtcctgtgtctagtgtgagttcttgacaagagtgtttttcccttcccgtcacagagtgggcccaacgagctacggcactttgaccccgagtttaccgaagagcctgtccccaactccattggcaagtcccctgacagcgtcctcgtcacagccagcgtcaaggaagctgccgaggctttcctaggcttttcctatgcgcctcccacggactctttcctctgaaccctgttagggcttggttttaaaggattttatgtgtgtttccgaatgttttagttagccttttggtggagccgccagctgacaggacatcttacaagagaatttgcacatctctggaagcttagcaatcttattgcacactgttcgctggaattttttgaagagcacattctcctcagtgagctcatgaggttttcatttttattcttccttccaacgtggtgctatctctgaaacgagcgttagagtgccgccttagacggaggcaggagtttcgttagaaagcggacctgttctaaaaaaggtctcctgcagatctgtctgggctgtgatgacgaatattatgaaatgtgccttttctgaagagattgtgttagctccaaagcttttcctatcgcagtgtttcagttctttattttcccttgtggatatgctgtgtgaaccgtcgtgtgagtgtggtatgcctgatcacagatggattttgttataagcatcaatgtgacacttgcaggacactacaacgtgggacattgtttgtttcttccatatttggaagataaatttatgtgtagacttttttgtaagatacggttaataactaaaatttattgaaatggtcttgcaatgactcgtattcagatgcctaaagaaagcattgctgctacaaatatttctatttttagaaagggtttttatggaccaatgccccagttgtcagtcagagccgttggtgtttttcattgtttaaaatgtcacctgtaaaatgggcattatttatgtttttttttttgcattcctgataattgtatgtattgtataaagaacgtctgtacattgggttataacactagtatatttaaacttacaggcttatttgtaatgtaaaccaccattttaatgtactgtaattaacatggttataatacgtacaatccttccctcatcccatcacacaactttttttgtgtgtgataaactgattttggtttgcaataaaaccttgaaaaatatttacatatattgtgtcatgtgttattttgtatattttggttaagggggtaatcatgggttagtttaaaattgaaaaccatgaaaatcctgctgtaatttcctgcttagtggtttgctccaacagcagtggtttctgactccagggagtataggatggcttaagccaccacgtccaggcctttagcagcatt

SEQ ID NO: 38 - DNA, 3612 bp, Homo sapiens
  source 1..3612: mol_type = other DNA
agaggcgtctgtagggtaaagctgggggttctgtagccggaggcggcggcgagtccagaacgtcctggccttacagggagaaggcgtcactcgcggttacaagtgcctgaccctcactccagttggcggaggaggagaaggaaggggccgggccgggtcccctcccctcgcgccccggatggatgtgcccggcccggtgtctcggcgggcggcggcggcggcggccactgtgctcctgcggaccgctcgggtccgtcgcgaatgctggttcttgccgaccgcgctgctctgcgcctacggcttcttcgccagcctcaggccgtccgagcccttcctgaccccgtacctgctggggccggacaagaacctgaccgagagggaggtcttcaatgaaatttatccagtatggacttactcttacctggtgctactgtttcctgtgttccttgccacagactacctccgttataaacctgttgttctactgcaggggctcagccttattgttacatggtttatgctgctctatgcccagggactgctggccattcaatttctagaatttttttatggcatcgccacagccactgaaattgcctattactcttatatctacagtgtggtggacctgggcatgtaccagaaagtcacaagttactgtcgaagtgccactttggtgggctttacagtgggctctgtcctagggcaaatccttgtctcagtggcaggctggtcgctgttcagcctgaatgtcatctctcttacctgtgtttcagtggcttttgctgtggcctggtttttacctatgccacagaagagcctcttctttcaccacattccttctacctgccagagagtgaatggcatcaaggtacaaaatggtggcattgttactgacaccccagcttctaaccaccttcctggctgggaggacattgagtcaaaaatccctctaaatatggaggagcctcccgtggaggaaccggaacccaagccagaccgtctccttgtattgaaagtactatggaatgatttcctgatgtgctactcctctcgccctcttctctgctggtctgtgtggtgggccctctctacctgtggctattttcaagttgtgaactacacacagggcctgtgggagaaagtgatgccttctcgctatgctgctatctataatggtggcgtggaggccgtttcaaccttactgggtgctgttgctgtgtttgcagttggttatataaaaatatcctggtcaacttggggagaaatgacattatctctcttttctctcctgattgctgctgcagtgtatatcatggacactgtgggtaacatttgggtgtgctatgcatcctatgttgtcttcagaatcatctacatgttactcatcacgatagcaacttttcaaattgctgcaaacctcagcatggaacgctatgccctagtatttggtgtaaataccttcattgccctggcactgcagacgctgctcactctaattgtggtagatgccagtggccttggattagaaattaccactcagtttttgatctatgccagttattttgcactcatcgctgtggttttcctggccagtggtgcagtcagtgttatgaagaaatgtagaaagctggaagatccacaatcaagttctcaagtaaccacttcataatatactgctgaagggcttcttcttatagcaagaactctgcacagcaactgcctggatgtgtttgattttttaaagcgtagacatatatttatgaatgtgcatttcttgacttcacagcagccacttgactaataccttgtgttccgggaataacatgatactattcagaggagccagaagtaaagtttatttcatggattatttatgagagctaatttaaggatgactttttttctgattcaaaagtgaacttgattttaaaaaccagtcaagagcaatcaaagcagcacatggtgttgtatacttcattagcaagtgagtttggtgttttataggtcacatatgtctgtatctacttagccagatgcttggcctggtgggaccagggctccacagaggccacaaaatgttgcaagtcatgatggatggaaatatgttctaacagcatctgcctctattcaatttaattcttatttctgtgttactcatgtacattggtctttctacatagttattctatcactggcaatatttgttctggtttagtgttctgtattttaaggtgtacgtatcatttctaattttaagttattttaaaaaaattcatcatatgaatgttcttggttcccattgtgacgattatttatttctgtaaaatttgtttagaagtacgtttttgcattattcatatgcttcccagagaagctcatttagttagaaaataaggcaagttttgaagcctgctaaatgaagagacttaagaaagcttaaggtacgcttgcttgtctttaaatcttcaatatgaaggactattaattccaagattaaaagttcatatataggctaaagatgtaactaggccatttgtatttgtattcccttttatttccaaaataaaatgaaaaatctttttttaataatttcatccctatttatagtttttatattaatttgtttttcttatccaagtaaagatgtcaataggaattgcattagtccaaggcctttttcataaactgagcctcttttcaattatttcaatgggacaggaactaggatagatgtgattcctgcatttttttaccttaaatctgcctttgtttctaaaggtagatcatcttgaatatttgcttaaaattgctagtgatttcattaccaagttacttgaaaaaatgttctatatgcatttaattctgaaatcagtctaccaaggggctgctagtatatgtcagacatgaaaactattttaaagctgactttgttgccttatcttgaaaagaatctagataggtgcttttaactggggtattaacttttttagaatgacacagctgaacagtgttaataatagtgtgtcaagattgcaaagtcgacatactcatttggtttaagcaggaatcctagaagcaaatggatggggataagaataggtcattttctattcaccatcctttactattaagggaaaggaaaagaacactagctaaggaagggaaagggaagtgatatcataaaagtagcaaccttcattttacattctgtctgtttttctttttttgctttgttttgtttgtgctaatttgggaattgtgtactccgaaacaagtagaaaagtgctgtttgagggattttattaaatctttttttaatggaatgtggtacaaattgttcatgttaccaaagcaatatttccctggaatttaattcaaagtttgtggcatacaacctgagccttttcttatataagacaagaatatgttcacatcttggtatgtggccatatttatagaatgctgaactcaatgtgcaagttgtactgtatgcagttttgtaaataagtgaaaataatttgttgtactttttattcaattctgtatagattataaaattatttttattaaataaatattttacagtatattttctctttttaatcaaaa

SEQ ID NO: 39 - DNA, 1217 bp, Homo sapiens
  source 1..1217: mol_type = other DNA
acatttcctaatcaagaagttggcgtgcagctgggagagctagactaagttggtcatgatgcagaagctactcaaatgcagtcggcttgtcctggctcttgccctcatcctggttctggaatcctcagttcaaggttatcctacgcggagagccaggtaccaatgggtgcgctgcaatccagacagtaattctgcaaactgccttgaagaaaaaggaccaatgttcgaactacttccaggtgaatccaacaagatcccccgtctgaggactgacctttttccaaagacgagaatccaggacttgaatcgtatcttcccactttctgaggactactctggatcaggcttcggctccggctccggctctggatcaggatctgggagtggcttcctaacggaaatggaacaggattaccaactagtagacgaaagtgatgctttccatgacaaccttaggtctcttgacaggaatctgccctcagacagccaggacttgggtcaacatggattagaagaggattttatgttataaaagaggattttcccaccttgacaccaggcaatgtagttagcatattttatgtaccatggttatatgattaatcttgggacaaagaattttatagaaatttttaaacatctgaaaaagaagcttaagttttatcatccttttttttctcatgaattcttaaaggattatgctttaatgctgttatctattttattgttcttgaaaatacctgcattttttggtatcatgttcaaccaacatcattatgaaattaattagattcccatggccataaaatggctttaaagaatatatatatatttttaaagtagcttgagaagcaaattggcaggtaatatttcatacctaaattaagactctgacttggattgtgaattataatgatatgccccttttcttataaaaacaaaaaaaaaaataatgaaacacagtgaatttgtagagtgggggtatttgacatattttacagggtggagtgtactatatactattacctttgaatgtgtttgcagagctagtggatgtgtttgtctacaagtatgattgctgttacataacaccccaaattaactcccaaattaaaacacagttgtgctgtcaatacctcatactgctttacctttttttcctggatatctgtgtattttcaaatgttactatatattaaagcagaaatataaccaaa

SEQ ID NO: 40 - DNA, 1827 bp, Homo sapiens
  source 1..1827: mol_type = other DNA
agacggagcccggacctccacactgagccatgcccacccccgacgccaccacgccacaggccaagggcttccgcagggccgtgtctgagctggacgccaagcaggcagaggccatcatgtccccgcggttcattgggcgcaggcagagcctcatcgaggacgcccgcaaggagcgggaggcggcggtggcagcagcggccgctgcagtcccctcggagcccggggaccccctggaggctgtggcctttgaggagaaggaggggaaggccgtgctaaacctgctcttctccccgagggccaccaagccctcggcgctgtcccgagctgtgaaggtgtttgagacgtttgaagccaaaatccaccatctagagacccggcccgcccagaggccgcgagctgggggcccccacctggagtacttcgtgcgcctcgaggtgcgccgaggggacctggccgccctgctcagtggtgtgcgccaggtgtcagaggacgtgcgcagccccgcggggcccaaggtcccctggttcccaagaaaagtgtcagagctggacaagtgtcatcacctggtcaccaagttcgaccctgacctggacttggaccacccgggcttctcggaccaggtgtaccgccagcgcaggaagctgattgctgagatcgccttccagtacaggcacggcgacccgattccccgtgtggagtacaccgccgaggagattgccacctggaaggaggtctacaccacgctgaagggcctctacgccacgcacgcctgcggggagcacctggaggcctttgctttgctggagcgcttcagcggctaccgggaagacaatatcccccagctggaggacgtctcccgcttcctgaaggagcgcacgggcttccagctgcggcctgtggccggcctgctgtccgcccgggacttcctggccagcctggccttccgcgtgttccagtgcacccagtatatccgccacgcgtcctcgcccatgcactcccctgagccggactgctgccacgagctgctggggcacgtgcccatgctggccgaccgcaccttcgcgcagttctcgcaggacattggcctggcgtccctgggggcctcggatgaggaaattgagaagctgtccacgctgtactggttcacggtggagttcgggctgtgtaagcagaacggggaggtgaaggcctatggtgccgggctgctgtcctcctacggggagctcctgcactgcctgtctgaggagcctgagattcgggccttcgaccctgaggctgcggccgtgcagccctaccaagaccagacgtaccagtcagtctacttcgtgtctgagagcttcagtgacgccaaggacaagctcaggagctatgcctcacgcatccagcgccccttctccgtgaagttcgacccgtacacgctggccatcgacgtgctggacagcccccaggccgtgcggcgctccctggagggtgtccaggatgagctggacacccttgcccatgcgctgagtgccattggctaggtgcacggcgtccctgagggcccttcccaacctcccctggtcctgcactgtcccggagctcaggccctggtgaggggctgggtcccgggtgccccccatgccctccctgctgccaggctcccactgcccctgcacctgcttctcagcgcaacagctgtgtgtgcccgtggtgaggttgtgctgcctgtggtgaggtcctgtcctggctcccagggtcctgggggctgctgcactgccctccgcccttccctgacactgtctgctgccccaatcaccgtcacaataaaagaaactgtggtctcta

SEQ ID NO: 41 - DNA, 4040 bp, Homo sapiens
  source 1..4040: mol_type = other DNA
atgtcagaggctgcctcgcaggggctgcgcgcagcggcaagaagtgtctgggctgggacggacaggagaggctgtcgccatcggcgtcctgtgcccctctgctccggcacggccctgtcgcagtgcccgcgctttccccggcgcctgcacgcggcgcgcctgggtaacatgcttggggtcctggtccttggcgcgctggccctggccggcctggggttccccgcacccgcagagccgcagccgggtggcagccagtgcgtcgagcacgactgcttcgcgctctacccgggccccgcgaccttcctcaatgccagtcagatctgcgacggactgcggggccacctaatgacagtgcgctcctcggtggctgccgatgtcatttccttgctactgaacggcgacggcggcgttggccgccggcgcctctggatcggcctgcagctgccacccggctgcggcgaccccaagcgcctcgggcccctgcgcggcttccagtgggttacgggagacaacaacaccagctatagcaggtgggcacggctcgacctcaatggggctcccctctgcggcccgttgtgcgtcgctgtctccgctgctgaggccactgtgcccagcgagccgatctgggaggagcagcagtgcgaagtgaaggccgatggcttcctctgcgagttccacttcccagccacctgcaggccactggctgtggagcccggcgccgcggctgccgccgtctcgatcacctacggcaccccgttcgcggcccgcggagcggacttccaggcgctgccggtgggcagctccgccgcggtggctcccctcggcttacagctaatgtgcaccgcgccgcccggagcggtccaggggcactgggccagggaggcgccgggcgcttgggactgcagcgtggagaacggcggctgcgagcacgcgtgcaatgcgatccctggggctccccgctgccagtgcccagccggcgccgccctgcaggcagacgggcgctcctgcaccgcatccgcgacgcagtcctgcaacgacctctgcgagcacttctgcgttcccaaccccgaccagccgggctcctactcgtgcatgtgcgagaccggctaccggctggcggccgaccaacaccggtgcgaggacgtggatgactgcatactggagcccagtccgtgtccgcagcgctgtgtcaacacacagggtggcttcgagtgccactgctaccctaactacgacctggtggacggcgagtgtgtggagcccgtggacccgtgcttcagagccaactgcgagtaccagtgccagcccctgaaccaaactagctacctctgcgtctgcgccgagggcttcgcgcccattccccacgagccgcacaggtgccagatgttttgcaaccagactgcctgtccagccgactgcgaccccaacacccaggctagctgtgagtgccctgaaggctacatcctggacgacggtttcatctgcacggacatcgacgagtgcgaaaacggcggcttctgctccggggtgtgccacaacctccccggtaccttcgagtgcatctgcgggcccgactcggcccttgcccgccacattggcaccgactgtgactccggcaaggtggacggtggcgacagcggctctggcgagcccccgcccagcccgacgcccggctccaccttgactcctccggccgtggggctcgtgcattcgggcttgctcataggcatctccatcgcgagcctgtgcctggtggtggcgcttttggcgctcctctgccacctgcgcaagaagcagggcgccgccagggccaagatggagtacaagtgcgcggccccttccaaggaggtagtgctgcagcacgtgcggaccgagcggacgccgcagagactctgagcggcctccgtccaggagcctggctccgtccaggagcctgtgcctcctcacccccagctttgctaccaaagcaccttagctggcattacagctggagaagaccctccccgcaccccccaagctgttttcttctattccatggctaactggcgagggggtgattagagggaggagaatgagcctcggcctcttccgtgacgtcactggaccactgggcaatgatggcaattttgtaacgaagacacagactgcgatttgtcccaggtcctcactaccgggcgcaggagggtgagcgttattggtcggcagccttctgggcagaccttgacctcgtgggctagggatgactaaaatatttattttttttaagtatttaggtttttgtttgtttcctttgttcttacctgtatgtctccagtatccactttgcacagctctccggtctctctctctctacaaactcccacttgtcatgtgacaggtaaactatcttggtgaatttttttttcctagccctctcacatttatgaagcaagccccacttattccccattcttcctagttttctcctcccaggaactgggccaactcacctgagtcaccctacctgtgcctgaccctacttcttttgctcttagctgtctgctcagacagaacccctacatgaaacagaaacaaaaacactaaaaataaaaatggccatttgctttttcaccagatttgctaatttatcctgaaatttcagattcccagagcaaaataattttaaacaaaggttgagatgtaaaaggtattaaattgatgttgctggactgtcatagaaattacacccaaagaggtatttatctttacttttaaacagtgagcctgaattttgttgctgttttgatttgtactgaaaaatggtaattgttgctaatcttcttatgcaatttccttttttgttattattacttatttttgacagtgttgaaaatgttcagaaggttgctctagattgagagaagagacaaacacctcccaggagacagttcaagaaagcttcaaactgcatgattcatgccaattagcaattgactgtcactgttccttgtcactggtagaccaaaataaaaccagctctactggtcttgtggaattgggagcttgggaatggatcctggaggatgcccaattagggcctagccttaatcaggtcctcagagaatttctaccatttcagagaggccttttggaatgtggcccctgaacaagaattggaagctgccctgcccatgggagctggttagaaatgcagaatcctaggctccaccccatccagttcatgagaatctatatttaacaagatctgcagggggtgtgtctgctcagtaatttgaggacaaccattccagactgcttccaattttctggaatacatgaaatatagatcagttataagtagcaggccaagtcaggcccttattttcaagaaactgaggaattttctttgtgtagctttgctctttggtagaaaaggctaggtacacagctctagacactgccacacagggtctgcaaggtctttggttcagctaagctaggaatgaaatcctgcttcagtgtatggaaataaatgtatcatagaaatgtaacttttgtaagacaaaggttttcctcttctattttgtaaactcaaaatatttgtacatagttatttatttattggagataatctagaacacaggcaaaatccttgcttatgacatcacttgtacaaaataaacaaataacaatgtgctctcgggttgtgtgtctgttcacttttcctccctcagtgccctcattttatgtcattaaatggggctcacaaaccatgcaaatgctatgagatgcatggagggctgccctgtaccccagcacttgtgttgtctggtggtggcaccatctctgattttcaaagctttttccagaggctattattttcactgtagaatgatttcatgctatctctgtgtgcacaaatatttattttctttctgtaaccataacaacttcatatatgaggacttgtgtctctgtgcttttaaatgcataaatgcattataggatcatttgttggaatgaattaaataaacccttcctggggcatctggcgaatccca

SEQ ID NO: 42 - DNA, 2269 bp, Homo sapiens
  source 1..2269: mol_type = other DNA
ctccacaggtccgccccaatccccgctcacacttgggaaacttgggactgcgctggggccgcgtgtggcacctcaggggggcggcccccggcctcaagaggagggggaggagaaggaggaagaggaggaagtgagcccgaaggatccgctcggagctgtttgtccagctgtttctattcgcacccggagcagtacagccagaagggggccgagccgaaggtggctggctttaggcgctaatttccaactcttttcctcacagcttgtcttttccaggcaccctggagtcccctcaggccagctcggtgggcgcgcacctgccagccgcccctgacctcgcaggccaggcgacctccgagcctgagaagatggcccagtccaagctcgattgccgctcacctgtcggcctcgactgctgcaactgctgcctggacctggcccatcggagtgggctccagcgaggcagcagcggggagaacaacaacccgggcagccctacagtgagcaactttcggcagctgcaggaaaagctggtctttgagaacctcaataccgacaagctcaacagcataatgcggcaggattcgctagagccggtgctgcgggacccctgctacctgatcaacgagggcatctgcaaccgcaacatcgaccagaccatgctctccatcctgctcttcttccacagtgcctccggagccagcgtggtggccatagacaacaagatcgaacaggccatggatctggtgaagaatcatctgatgtatgctgtgagagaggaggtggagatcctgaaggagcagatccgagagctggtggagaagaactcccagctagagcgtgagaacaccctgttgaagaccctggcaagcccagagcagctggagaagttccagtcctgtctgagccctgaagagccagctcccgaatccccacaagtgcccgaggcccctggtggttctgcggtgtaagtggctctgtcctcagggtgggcagagccactaaacttgttttacctagttctttccagtttgtttttggctccccaagcatcatctcacgaggagaactttacacctagcacagctggtgccaagagatgtcctaaggacatggccacctgggtccactccagcgacagacccctgacaagagcaggtctctggaggctgagttgcatggggcctagtaacaccaagccagtgagcctctaatgctactgcgccctgggggctcccagggcctgggcaacttagctgcaactggcaaaggagaagggtagtttgaggtgtgacaccagtttgctccagaaagtttaaggggtctgtttctcatctccatggacatcttcaacagcttcacctgacaacgactgttcctatgaagaagccacttgtgttttaagcagaggcaacctctctcttctcctctgtttcgtgaaggcaggggacacagatgggagagattgagccaagtcagccttctgttggttaatatggtataatgcatggctttgtgcacagcccagtgtgggattacagctttgggatgaccgcttacaaagttctgtttggttagtattggcatagtttttctatatagccataaatgcgtatatatacccatagggctagatctgtatcttagtgtagcgatgtatacatatacacatccacctacatgttgaagggcctaaccagccttgggagtattgactggtcccttacctcttatggctaagtctttgactgtgttcatttaccaagttgacccagtttgtcttttaggttaagtaagactcgagagtaaaggcaaggaggggggccagcctctgaatgcggccacggatgccttgctgctgcaaccctttccccagctgtccactgaaacgtgaagtcctgttttgaatgccaaacccaccattcactggtgctgactacatagaatggggttgagagaagatcagtttgggcttcacagtgtcatttgaaaacgttttttgttttgttttgtaattattgtggaaaactttcaagtgaacagaaggatggtgtcctactgtggatgagggatgaacaaggggatggctttgatccaatggagcctgggaggtgtgcccagaaagcttgtctgtagcgggttttgtgagagtgaacactttccactttttgacaccttatcctgatgtatggttccaggatttggattttgattttccaaatgtagcttgaaatttcaataaactttgctctgtttttctaaaaataaa

SEQ ID NO: 43 - DNA, 2815 bp, Homo sapiens
  source 1..2815: mol_type = other DNA
agagcgaggtggtgaggagagctggttgcgtgagtctcctcagctctgcttaccggtgcgactagcggcagcgacgcggctaaaagcgaaggggcgagtgcgagtcccctgagctgtacgaacgcggtcgccatggaccgcccagatgaggggcctccggccaagacccgccgcctgagcagctccgagtctccacagcgcgacccgcccccgccgccgccgccgccgccgctcctccgactgccgctgcctccaccccagcagcgcccgaggctccaggaggaaacggaggcggcacaggtgctggccgatatgaggggggtgggactgggccccgcgctgcccccgccgcctccctatgtcattctcgaggagggggggatccgcgcatacttcacgctcggtgctgagtgtcccggctgggattctaccatcgagtcggggtatggggaggcgcccccgcccacggagagcctggaagcactccccactcctgaggcctcgggggggagcctggaaatcgattttcaggttgtacagtcgagcagttttggtggagagggggccctagaaacctgtagcgcagtggggtgggcgccccagaggttagttgacccgaagagcaaggaagaggcgatcatcatagtggaggatgaggatgaggatgagcgggagagtatgaggagcagcaggaggcggcggcggcggcggaggaggaagcagaggaaggtgaagagggaaagcagagagagaaatgccgagaggatggagagcatcctgcaggcactggaggatattcagctggatctggaggcagtgaacatcaaggcaggcaaagccttcctgcgtctcaagcgcaagttcatccagatgcgaagacccttcctggagcgcagagacctcatcatccagcatatcccaggcttctgggtcaaagcattcctcaaccaccccagaatttcaattttgatcaaccgacgtgatgaagacattttccgctacttgaccaatctgcaggtacaggatctcagacatatctccatgggctacaaaatgaagctgtacttccagactaacccctacttcacaaacatggtgattgtcaaggagttccagcgcaaccgctcaggccggctggtgtctcactcaaccccaatccgctggcaccggggccaggaaccccaggcccgtcgtcacgggaaccaggatgcgagccacagctttttcagctggttctcaaaccatagcctcccagaggctgacaggattgctgagattatcaagaatgatctgtgggttaaccctctacgctactacctgagagaaaggggctccaggataaagagaaagaagcaagaaatgaagaaacgtaaaaccaggggcagatgtgaggtggtgatcatggaagacgcccctgactattatgcagtggaagacattttcagcgagatctcagacattgatgagacaattcatgacatcaagatctctgacttcatggagaccaccgactacttcgagaccactgacaatgagataactgacatcaatgagaacatctgcgacagcgagaatcctgaccacaatgaggtccccaacaacgagaccactgataacaacgagagtgctgatgaccacgaaaccactgacaacaatgagagtgcagatgacaacaacgagaatcctgaagacaataacaagaacactgatgacaacgaagagaaccctaacaacaacgagaacacttacggcaacaacttcttcaaaggtggcttctggggcagccatggcaacaaccaggacagcagcgacagtgacaatgaagcagatgaggccagtgatgatgaagataatgatggcaacgaaggtgacaatgagggcagtgatgatgatggcaatgaaggtgacaatgaaggcagcgatgatgacgacagagacattgagtactatgagaaagttattgaagactttgacaaggatcaggctgactacgaggacgtgatagagatcatctcagacgaatcagtggaagaagagggcattgaggaaggcatccagcaagatgaggacatctatgaggaaggaaactatgaggaggaaggaagtgaagatgtctgggaagaaggggaagattcggacgactctgacctagaggatgtgcttcaggtcccaaacggttgggccaatccggggaagagggggaaaaccggataagggttttccccttttggggatcacctctctgtatcccccacccactatcccatttgccctcctcctcagctagggccacgcggccccacattgcacttctggggggtgaccgacttcgtacacgggtttaaagtttatttttatggtttagtcattgcagagttcttattttggggggagggaaagggggctagtccccttcttttggccctccgcccccgcaggcttctgtgtgctgctaactgtatttattgtgatgccttggtcagggcccctctacccacttctcccagtcagttgtggccccagcccctctccctgtgctgtgtggagtggacaccctgacccccgaagcggggagggccgctgtggccttcgtcacagccgcgcagtgcccatggaggcgctgctgccaccttcctctcccaagttctttctccatccctctcctcttcccgccgcgccgctagcccgcctcggtgtctatgcaaggccgcttcgccattgcggtattctttgcggtattcttgtccccgtcccccagaaggctcgcctctccccgtggaccctgttaatcccaataaaattctgagcaagttca

SEQ ID NO: 44 - DNA, 2654 bp, Homo sapiens
  source 1..2654: mol_type = other DNA
aggactcggccccatggagacgccgccggccgccgctggcgcatggcgggtaggagctgtggcgcggggccttccaggagtctgagctatgagtggcccctgtggagagaagcctgtcctggaagccagccccaccatgagtctgtgggaatttgaggacagccacagccgtcagggcaccccaaggccgggtcaagagctggccgctgaggaggcctcggccctggaactgcagatgaaggtggacttcttccggaagctgggctattcatccacggagatccacagcgtcctgcagaagctgggcgtccaggcagacaccaacacggtgctgggtgagctggtgaaacacgggacagccaccgagcgggagcgccagacctcaccggacccctgccctcagctccctctagtcccgcggggtggtggcacccctaaggctcccaacctggagcctccactcccagaagaggaaaaggagggcagcgacctgagaccagtggtcatcgatgggagcaacgtggccatgagccatgggaacaaggaggtcttctcctgccggggcatcctgctggcagtgaactggtttctggagcggggccacacagacatcacagtgtttgtgccatcctggaggaaggagcagcctcggcccgacgtgcccatcacagaccagcacatcctgcgggaactggagaagaagaagatcctggtgttcacaccatcacgacgcgtgggtggcaagcgggtggtgtgctatgacgacagattcattgtgaagctggcctacgagtctgacgggatcgtggtttccaacgacacataccgtgacctccaaggcgagcggcaggagtggaagcgcttcatcgaggagcggctgctcatgtactccttcgtcaatgacaagtttatgccccctgatgacccactgggccggcacgggcccagcctggacaacttcctgcgtaagaagccactcactttggagcacaggaagcagccgtgtccctatggaaggaaatgcacctatgggatcaagtgccgattcttccacccagagcggccaagctgcccccagcgctctgtggcagatgagctccgtgccaatgctctcctctcaccccccagagccccaagcaaggacaaaaatggccggcggccttcaccttcatcccagtccagctctctgctaacagagagtgagcagtgcagcctggatgggaagaagctgggggcccaggcatccccagggtcccgccaagagggtctaacacagacctatgccccatcaggcaggagcctcgcacctagcgggggcagtggcagcagctttgggcccacagactggctcccacagacgctggactcactcccgtacgtctcccaggattgcctggactcgggcattggctccctggagagccagatgtcggaactttggggggttcgaggaggaggccctggtgagccgggcccaccccgagccccttacacgggctacagtccctatggatctgagctcccagccaccgcagccttctctgcctttggccgggccatgggtgctggccacttcagtgtccctgccgactacccacccgcgccccctgcctttccacctcgagagtactggtctgaaccatacccactgcccccacccacatcagtccttcaggagcccccagtgcagagcccaggggctggcaggagcccgtggggcagggcaggcagcctggccaaggagcaggccagcgtgtatactaagctgtgtggtgtgtttcccccgcacctggtggaggctgtgatggggcgcttcccacagctcctggacccccagcagctggctgccgagatcctctcctacaagtcccagcaccccagtgagtaagctgcctgtggctggcaagggcagcacccccagcctccaagggccgtcaggctgggctttgggccattgagcagcccattcccagccctgaggcccaccccagaggctggacagagggaggattcaagtcgggaaggaaacccacaaaccaaagatactgtaggattggttctggcccatgcagcacctctagctgtctgcctcagtgggtcagaagcgatcaccctgttgatacacattgtatctctgtagtttaaggagacgctgccggtaacggcgtcggtccgtggctgaggcccaaaccgtcttttctctcagagggtggggagggaggtgggggcagcagaggcctgggctgggtgccctgtgcacgccaccccacttccgccctacccctgggacgttggccttggctggctagttgggcaccgtgtgcctgccctccaagggcctcctctacgccaatgaggcctcatctgtgctctcgctgggcacgtggcttcatgtcagtaagcaagatgcttcttaataacccaccttctgccccactctattccttatcctgctgcccctgtaggggtcaagggccctccgtctacaccctcttcttctcctccatcctttattcagagtcatctcgcccttccccatgggtgggggaacctgtgtttgtttgtgtgcacatgtaaattttaaatattttaagcagaaagtccttacctcctgtaacacatcaataaagtacaatcattgtgagccctttcaa

SEQ ID NO: 45 - DNA, 5841 bp, Homo sapiens
  source 1..5841: mol_type = other DNA
gttagtcggccgggacgcggagctgtgtgcatctcctacgcgggctcgctcgctcccgggagccgcgcagcttccccggcggtggcaggagcggcgaagagcgccatcagcgggcccttacggccccaggcctcgcggcgcgcggtccgctcgccctcctcgtccgcccaacctgcggcctgtggaggcgtgaggcgacgacaggcgagcgcctcgagcagcgttagccgctgcggccgccggtcctccctccacctcctcctcggccccccctcgcttccctcctcccacttcccgagctccggcgtgtcccggccacgctcgacgctgctgcaggaacaaaggaagaccccgcggcggcggcggcgccacctccgcctgctgctccgacccgctcccggcccgcggcggcggcaccagggcgcccggctcagccttcccggaggcctcggcccggcctcatcgtgccggcttcgcgcgcgaacccggctttcgcatttgggaccctgcagggccctgaaaaggagcctttaaaagtcaattctactccatttggcaaagagggaactgaaatctggaaagtaaggacttgcccaaggtcactgacttagtggcagactagagacttgaactcaggtctccattcccagcagaaaaatatggctcaggagactaaccagaccccggggcccatgctgtgtagcacaggatgtggcttttatggaaatcctaggacaaatggaatgtgttcagtttgctacaaagaacatcttcagaggcagcaaaatagtggcagaatgagcccaatgggaacagctagtggttccaacagtcctacctcagattctgcatctgtacagagagcagacactagcttaaacaactgtgaaggtgctgctggcagcacatctgaaaaatcaagaaatgtgcctgtggctgccttgcctgtaactcagcaaatgacagaaatgagcatttcaagagaggacaaaataactaccccgaaaacagaggtgtcagagccagttgtcactcagcccagtccatcagtttctcagcccagtacttctcagagtgaagaaaaagctcctgaattgcccaaaccaaagaaaaacagatgtttcatgtgcagaaagaaagttggtcttacagggtttgactgccgatgtggaaatttgttttgtggacttcaccgttactctgacaagcacaactgtccgtatgattacaaagcagaagctgcagcaaaaatcagaaaagagaatccagttgttgtggctgaaaaaattcagagaatataaattacttcttgtgaagagactgaaactttgtttttattttaatatatcgtaggaaaacattaaagagcagatgcatggccatttttctttgatgttctccagagttttacattacacttgtctgtcttataattgatattttaggatgtttgggtgtttgttacaggcagaattggatagatacagccctacaaatgtatatgccctcccctgaaaaaaattggatgaaaatctgcacagcaaagtgaaacacacagataataggaacaaaatgtagttcccatgtgccaaacaaaataaatgaaatctctgcatgtttgcagcatatctgccttttgggaatgtaatcaaggtataatctttggctagtgttatgtgcctgtatttttttaaaatggtacaccagaaaaggactggcagtctacttctaccatagttaaacttcaccctctttaatttcacaacatattctttggaagcaggaagaaatgctcataaagaggatcagaccttctttcccgtgaaaccagtatttggcgccatatataagcctggttaaattggtcatctaaagctgtcaaataagacattctgtgaaaggtaaacatcgaaactggttataagtaaaaccatcaagccaacaacagggtcttgagataacctttgaagcttattgtactggcctgcaccagaagatgtctgcattactcattgctaaaaatgtgtagcacagaactgcactaggattaatttgtttacaagaagaaatttaaactctacgtttggttttcacatacagcagctctattgaataacatgcatctgaattttaagttgcaaaggtatctgaataatttttcatgtgcatcttttgtcgaatgttttggttcaagaaagaatgtttaaagctttttaaaagacttcagttcttaatgtaactgtacccttctgcatggaaaatcataaccaacatggctgcagtagacttcttagtggtatccagcaccacttgcagagggctgctttatcatattgtacttgggtgtaggactctagtgttcttgggtgtattgcatgggctgcattatctacagcattgtacaataacaactagaaaaggcagtatacttcactgatgcttgtctggtaataatcacttctgtgttataatggaaggttttttgtgatgtatgaaacttgtgttttttatatataaatgagtatagttagtgttgtggtaatgcctgttttcatctgtaaatagttaagtatgtacacgaggcactacttctgatttattgcaatgttcagtcctagtttttacttttattcttaaagcattcagttttgctttcaattttatgtaccttagttctgagttagacctgcagatgtgtacagatagttcatatttatgtattgcacataatcatgctattcagcattgatgctatattgtattatgtaaataataaaagccatgtacagagggaaacttcacttgttcattgggtttttaagccatagttggagtcctaaagggggaaaattagaaaatgttactttacatggctatataattttttttcctttgccatcgctgaacttgttcaagtgataatcaagagatattagaagcatttgcaggtatgatacctgataggcagtttatttttgaagaattcaataggcacctttgggattaaaacaattttaaaatttgagaaagatgaaatttgaacaattatggcctacctcgcttctccatgttgtaaaagatgatgttttatgacaattctgaagaagaaagtaggtatgacttagtttacaaaaagagttctgttttatttctgttttgggtttggctctaatacagagattcacccatggacttggtctctgtagcgtttatgccagatcataaagaacaaggggtgacaggtacatgttgactttacctggggtaaatgattaccagagtgcaactgttgcttctctgtgatggagtttggggtggaaataccctagttttatctgtcagttctttgcatgctgctacttttgcttcaagttagggaggtacatatgtatgtcaaagttcctatactgacatagggtagtatttctctgaatatgaaaagtcacaattgagttgaaaatttagaagttgaagatttagaagggcaacttaatttttcaagttaaaaatggaagtaagagtggtatagagaaggtaagatgagccctcggactccatcactctgaaggtctgagtagtagcaagagatagatttttgtttggccaggctgcagtgcaatggcgtaatcttggctcactgcaacctctgcctcctgggttcaagtgattctcctgcctctgcctcctaagtagctgggatcacaggcaccagccaccacgcccagctaatttttgtatttttaatagagacagtttctccatgttggtcaggctggtcttgaactcctgacctcaggtgagctgcccgcctcggcctcccaaagtgctgggattacaggcgtgaggcaccgcacccggccaatcagttttttttgttttttttttgtttgtttgttttacaagaaacattaaaccagatcagaacaaagatttgtcagggttgtccattgactggggagaaagggaatcagtgtgtaaggcacctgagaggactcagttctacccaaattcttgcattcaaacctgaatgcaaggccatgaagtcctttgaaatgtacagatattaagcaatttaataatataccatgtttcccttacataagcagacaaatatttacacccccatgacctgaagttttgttaggctgtgaccacatgaattcgcagtataaatgggtgagtcctttttgaaaacctcatcttcagtgtgcctgatctgtaaaactagagcctcaggtttgatcctaggtaggcttcccacaacgtggggaaaattagtttgtgagggaaagctgtttctgagcattatggcatagtgattaagatgagggaccctggaaccagcccgtgattttgggcaagttattttaggtccactttggactgcttcatctcttaaatgaagacagcaaccaccttcaggagtgtgaaggtgacaacacttaaaactgtgcctggcacattgtatttgctattgtcttaatgttctggaataatggtttccataacttgggaagaaaactttgagaacaggattggttacatcaagttgtatagtgaaaagataaagatttgttatttaaacttgcagtttaaagtcattagaatcattcttttccgtaaggctttctttgcaagagatctgcattaaataaagttgctaggaaataactaaaattggggaaataatctaataatagcaagatgttaagcatactattattgtattttgggggttggtaataacattcacatggatttatcaatacacactgagaagcaaagcctctcaagctgtcccatatcctccatttcaaaggcacacatacattttaggtaactcataatttagaaaggttatttaatcttttccacatgtaaatatttgaatatgtacaaagacttgatttgactcttgtctgtttttgttttgttttgtttgtttgagacagaggctccgtcgcccaggctggagtaaaatggcatggtctcagctcactgcaagttccgcctcccgggttcacgccattctcctggctcagcctccggagtagttgggactacaggcacccgtcaccacacctggctaatgttttttgttttttgggtttttttgtatttttagtagagagagggtttcaccgtgttagccagggtggtctcgatctcctgacctcatgatccgcccaccccggcctcccaaaatgctgggattacaggtgtgagccactgcgcctggccgactcttgtctgtttgaatgcaaagttctcaatagtggtttttgtccataagtattaacttataattttggagagtgataggtatggttgctggttgattagcaataagattatgtagccttaaactagctagaagagtttgcatgggtgaggcaacaggctgttaacaaaaagaaactggaaatacagtttcccctgtttgtttctcctgtttgtacaatctgctttaaaaacaagtgaacatgcaccatgtcagtcatggtgaatcggccaacagccagcccttgccagttgacatcacagtctaagatgggaaactggtacagatagacatgaagagagcttagcagtggttgaggtggtgactaaatatacagtcattgaataaataccgtgtagcaaatgtaaaaaaaaaaaaaaaagtacaacagaatacaaaagtgccatttaatattttatagctatttctatgcacaagtgctggttttaaatttatataaaagcaaagactgtttctgtgttttcttccagagtatttctgttacagccatagaagtaaagttaataaatgtaagactttttttgttttgttttgtgcca